(19) United States
(12) Patent Application Publication
(10) Pub. No.: US 2019/0085417 A1
(43) Pub. Date: Mar. 21, 2019

(54) Detection and Quantification of Target Nucleic Acid Sequence of a Microorganism

(71) Applicant: LUCENCE DIAGNOSTICS PTE LTD, Singapore (SG)

(72) Inventors: Min-Han Tan, Singapore (SG); Thu Hong Anh Vo, Singapore (SG)

(21) Appl. No.: 16/062,244

(22) PCT Filed: Dec. 19, 2016
(86) PCT No.: PCT/SG2016/050611

§ 371 Date: Jun. 14, 2018

(30) Foreign Application Priority Data
Dec. 18, 2015 (SG) 10201510448Q

(51) Int. Cl. C12Q 1/70; C12Q 1/689; C12Q 1/6895

(52) U.S. Cl. CPC C12Q 1/70; C12Q 1/689; C12Q 2600/158; C12Q 2600/112; C12Q 2600/118; C12Q 1/6895

(57) ABSTRACT

The present invention provides a method for detecting and/or quantifying the presence of a target nucleic acid sequence of a microorganism in a sample obtained from a subject, including amplifying the target sequence in a CpG island of the nucleic acid of the microorganism, irrespective of the methylation status of the CpG island. The invention is embodies by a method for detecting and/or quantifying the presence of a target nucleic acid sequence of Epstein-Barr virus (EBV) by amplifying a target sequence in the BamHI-W region of EBV in cell free DNAs (cfDNAs) obtained from a subject. The present invention also provides a kit to be used for the method of the invention.

### CROSS-REFERENCE TO RELATED APPLICATIONS

This application claims the benefit of priority of Singapore provisional application No. 10201510448Q, filed 18 Dec. 2015, the contents of it being hereby incorporated by reference in its entirety for all purposes.

### FIELD OF THE INVENTION

The present invention relates to molecular biology in particular biomarkers. In particular, the present invention relates to the detection and quantification of biomarkers associated with microorganisms such as bacteria, viruses, fungi and parasites, and methods of determining the likelihood that a patient suffers or is likely to suffer from diseases associated with microorganisms, and to predict the treatment outcome of a patient having diseases associated with microorganisms, by detecting and quantifying the biomarkers associated with the microorganisms.

### BACKGROUND OF THE INVENTION

Infections with microorganisms, including viruses, bacteria, fungi and parasites, have been recognized as risk factors for a wide range of diseases in humans, such as cancers, autoimmune diseases and cardiovascular diseases, which are associated with high mortality rates. Early detection of the causative microorganisms is crucial for the prevention, detection, diagnosis and treatment of these diseases associated with infections with microorganisms.

Examples of conventional methods used for the detection of microorganisms include culture-based procedures, serologic tests and microscopy. Each of these methods is associated with its own limitations. For example, culture-based procedures, which are dependent on the growth of the microorganisms, often takes days for results to become available, and such procedures often reveal false negative results due to the administration of an empiric antibiotic therapy. Serologic tests often face high false negative rate due to the absence or weakness of antibody production in the patient's body, or high false positive rate due to the presence of cross-reacting antibodies. Microscopy techniques may be limited by sample staining and fixing and the use of strong illumination, which may destroy or distort cellular features of the microorganisms to be detected.

Molecular diagnostic procedures such as PCR-based techniques have been introduced for the detection of microorganism infections in recent years. However, the currently available PCR-based techniques have some limitations in the quantitative measurement of the bacterial/viral load in patients' samples, as well as problems of false positive results. Thus, what is needed is an improved method of detecting and/or quantifying of microorganisms with better sensitivity and specificity.

### SUMMARY OF THE INVENTION

In one aspect, there is provided a method for detecting and/or quantifying the presence of a target nucleic acid sequence of a microorganism in a sample obtained from a subject, comprising amplifying the target sequence in a CpG island of the nucleic acid of the microorganism, irrespective of the methylation status of the CpG island.

In another aspect, there is provided a method for detecting and/or quantifying the presence of a target nucleic acid sequence of Epstein-Barr virus (EBV) in a sample obtained from a subject, comprising amplifying a target sequence in the BamHI-W region of EBV, wherein the target sequence comprises the sequence of

AGATCTAAGGCCGGGAGAGGCAGCCCCAAAGCGGGTGCAGTAACAGGTAATC TCTGGTAGTGATTTGGACCCGAAATCTGACACTTTAGAGCTCTGGAGGACTTTA AAACTCTAAAAATCAAAACTTTAGAGGCGAATGGGCG (SEQ ID NO: 1), wherein amplifying the target sequence comprises the use of a pair of oligonucleotide primers and a probe, wherein the first oligonucleotide primer comprises the sequence of 5′-AGATCTAAGGCCGGGAGAGG-3′ (SEQ ID NO:2), and the second oligonucleotide primer comprises the sequence of 5′-CGCCCATTCGCCTCTAAAGT-3′ (SEQ ID NO: 3), and wherein the probe comprises the sequence of 5′-(6-FAM)CTCTGGTAGTGATTTGGACCCGAAATCTG(TAMRA)-3′ (SEQ ID NO: 4), and wherein the method is a quantitative polymerase chain reaction (qPCR).

In yet another aspect, there is provided a method of detecting a disease associated with microorganism infection, or risk of developing a disease associated with microorganism infection in a subject, comprising detecting and/or quantifying the presence of a nucleic acid sequence of the microorganism using the method of the present invention in a sample obtained from the subject, wherein the presence of the nucleic acid sequence of the microorganism in the sample indicates that the subject has a disease associated with microorganism infection or is at risk of developing a disease associated with microorganism infection.

In another aspect, there is provided a method of detecting and treating a disease associated with microorganism infection, comprising: (i) detecting and/or quantifying the presence of a nucleic acid sequence of the microorganism using the method of the present invention in a sample obtained from the subject, wherein the presence of the nucleic acid sequence of the microorganism in the sample indicates that the subject has a disease associated with microorganism; and (ii) administering to the subject a medicament suitable for the treatment of the disease associated with the microorganism.

In yet another aspect, there is provided a method of predicting the treatment outcome of a disease associated with microorganism infection in a patient, comprising:(i) quantifying the nucleic acid sequence of the microorganism in a sample collected from the patient before treatment or before a treatment step, and quantifying the nucleic acid sequence of the microorganism in a sample collected from the same patient after treatment or after a treatment step; (ii) comparing the amount of the nucleic acid sequence of the microorganism in the sample before and after treatment or a treatment step, wherein a decrease in the amount of the nucleic acid sequence of the microorganism in the sample after treatment or a treatment step indicates that treatment outcome of the disease associated with microorganism infection in the patient is positive, wherein the quantifying of the nucleic acid sequence of the microorganism in the sample is performed according to the method of the present invention.

In a further aspect, there is provide a kit for detecting and/or quantifying the nucleic acid sequence of a microorganism in a sample obtained from a subject, comprising a pair of oligonucleotide primers specific for the amplification of a target sequence in a CpG island of the nucleic acid of the microorganism.

### BRIEF DESCRIPTION OF THE DRAWINGS

The invention will be better understood with reference to the detailed description when considered in conjunction with the non-limiting examples and the accompanying drawings, in which:

FIG. 1 depicts the overall survival curves of patients with nasopharyngeal carcinoma (NPC) with NPC levels measured using different circulating biomarkers. Results are shown using Kaplan-Meier plots for dichotomized biomarker variables. FIG. 1A shows the overall survival curves of NPC patients with EBV measured by the number of copies of BamHI-W per ml using qPCR. FIG. 1B shows the overall survival curves of NPC patients with EBV measured by the number of copies of EBNA1 per ml using qPCR. FIG. 1C shows the overall survival curves of NPC patients with EBV measured by the number of copies of EBNA1 per ml using dPCR. FIGS. 1D and 1E show the overall survival curves of NPC patients with NPC level measured in terms of the counts of circulating tumor cells (CTCs). The results show that the levels of BamHI-W (detected using dPCR) and EBNA1 (detected using dPCR or qPCR) serve as better indicators of the overall survival rate as compared to CTCs.

FIG. 2 is a flow chart depicting the overall results of detecting NPC using different EBV biomarkers. A total of 46 NPC patients, all of Asian ethnicity, took part in the study. The blood sample collected from each of these patients have been subjected to all three assays—BamHI-W qPCR assay, EBNA1 qPCR assay, and EBNA1 dPCR assay. As shown in the flow chart, BamHI-W qPCR assay detected 41 patients as NPC positive and 5 patients as NPC negative; EBNA1 qPCR assay detected 31 patients as NPC positive and 15 patients as NPC negative; EBNA1 dPCR assay detected 39 patients as NPC positive and 7 patients as NPC negative. The results indicate that the BamHI-W qPCR assay has better sensitivity for the detection of NPC as compared to EBNA1 qPCR and EBNA1 dPCR assays.

FIG. 3 is a flow chart depicting the process of designing and selecting the synthetic internal control. 50 random sequences with the length of 150 bases were generated using R program. Sequences with multiple base repeats and runs (i.e. ATATAT, CCCCCC, etc.) were eliminated. 15 sequences passed this criterion. Primers and probes were designed for these 15 sequences such that the target sequences are about 100 bp and located in the central region of the 150-base sequences. Primer-dimer and primer-probe dimer formation were checked by NetPrimer program. The ΔG values between all the primers and probe (in both Internal Control (IC) and BamHI-W reaction) must be more positive than −9 kcal/mole (see Table 1). Only one sequence qualified under this criterion, and this sequence was then checked for similarity in NCBI Genomic Reference Sequences, NCBI Chromosome Sequences and Nucleotide Collection using BLAST program. No significant similarity was found in all the databases, indicating the selected internal control sequence is unique.

TABLE 1
Values of ΔG between the primers and probe of selected
Internal Control (IC) and the primers and probe of BamHI-W.

| | | BamHI-W | | |
|---|---|---|---|---|
| ΔG | | Forward Primer | Reverse Primer | Probe |
| IC | Forward Primer | −7.24 | No | −8.26 |
| | Reverse Primer | No | −5.67 | −5.41 |
| | Probe | −5.67 | −3.94 | No |

FIG. 4 depicts the detection of EBV using BamHI-W qPCR assay, in plasma samples with and without the internal control (IC) plasmid. X-axis represents the copy number of BamHI-W detected, and Y-axis represents the threshold cycle (Ct) which is the intersection between an amplification curve and a threshold line. The results indicate that the internal control plasmid does not interfere with the detection of EBV using BamHI-W qPCR assay.

FIG. 5 illustrates the different experimental settings used to test the impact of the internal control (IC) plasmid on the detection of EBV using BamHI-W qPCR assay. In setting 1, no IC plasmid was added into the sample, and the qPCR was performed on the DNA sample after it was extracted In Setting 2, DNA was extracted from the sample first, and the IC plasmid was added into the extracted DNA sample. Both the IC and the EBV target sequence in the extract DNA were amplified by the qPCR. In Setting 3, the IC plasmid was added before the DNA extraction step. Both the IC and the EBV target sequence were extracted using the DNA extraction method, and amplified by the qPCR.

FIG. 6 shows examples of qPCR amplification curves with the internal control (IC) plasmid added in the sample to detect false negative qPCR amplifications. FIGS. 6A and 6B indicate positive results for EBV detection using BamHI-W qPCR assay. FIG. 6C indicates true negative result for EBV detection using BamHI-W qPCR assay, as the IC plasmid was amplified by the qPCR assay. FIG. 6D indicates false negative result for EBV detection using BamHI-W qPCR assay, as the IC plasmid was not amplified by the qPCR assay. Over all, it can be concluded that IC can serve as a control to identify a false negative detection of BamHI-W.

FIG. 7 depicts the construction of a BamHI-W standard plasmid and an internal control (IC) plasmid.

FIG. 8 shows the confirmation of the BamHI-W sequence in the BamHI-W standard plasmid and the internal control (IC) sequence in the IC plasmid constructed as depicted in FIG. 7. FIG. 8A shows the sequence of the BamHI-W standard forward strand. FIG. 8B shows the sequence of the IC forward strand. FIG. 8C shows the sequence of the BamHI-W standard reverse strand. FIG. 8D shows the sequence of the IC reverse strand. The results indicate that the BamHI-W sequence inserted in the BamHI-W plasmid has 100% sequence identity as the BamHI-W region of EBV, and that the IC sequence inserted in the IC plasmid has 10% sequence identity as the designed sequence of the IC.

FIG. 9 shows the results of plasmid validation in clinical samples using BamHI-W qPCR assay. FIG. 9A shows the results where the internal control (IC) plasmids were added in the plasma sample from the patient prior to DNA extraction. FIG. 9B shows the results of amplification of the constructed BamHI-W standard plasmids added directly to PCR plates. The results indicate that the constructed IC and BamHI-W plasmids can be amplified after being extracted together with DNA from clinical samples (FIG. 9A) and when being added directly in the PCR plates (FIG. 9B).

FIG. 10 shows the derivation of conversion factor between BamHI-W standard plasmids constructed and the International Standard (IU) of EBV defined by the National Institute for Biological Standards and Control (NIBSC). FIG. 10A shows the plot of the number of copies of BamHI-W plasmids added in the sample (y-axis) against the number of !Us defined by NIBSC (x-axis), both in log scale. FIG. 10B shows the plot of the number of copies of BamHI-W plasmids added in the sample (y-axis) against the number of IUs defined by NIBSC (x-axis). The conversion factor between BamHI-W standard plasmid and the NIBSC standard is 1 IU of EBV=1.38 copies of BamHI-W.

FIG. 11 shows the PCR amplification curves of the target sequences of Mycobacterium tuberculosis. In FIGS. 11A and 11C, the target sequence is nucleotide sequence from nucleotide position 1542511 to nucleotide position 1542349 of the genomic sequence of Mycobacterium tuberculosis. In FIGS. 11B and 11D, the target sequence is nucleotide sequence from nucleotide position 1542328 to nucleotide position 1542215 of the genomic sequence of Mycobacteriaum tuberculosis. FIGS. 11A and 11B represent the amplification curve using a sample from a patient known to have tuberculosis, and 11C and 11D represent the amplification curve using a sample from a healthy subject control. The results indicate that both target sequences of Mycobacterium tuberculosis are present in the sample from the patient with tuberculocis, but absent in the sample from the healthy subject control.

FIG. 12 shows a gel electrophoresis photo of using different sets of BamHI-W primers directed to different target sequences for the amplification of BamHI-W. An EBV-positive cell line, C666-1, was used as the positive control. EBV-negative RKO, a buffy coat of healthy donor was used as the negative control. The sequences of primer pair 2 are: forward primer 5′-GGAATAAGCCCCCAGACAGG-3′ (SEQ ID NO:12), reverse primer 5′- TTACGTAAACGCGCTGGACT-3′ (SEQ ID NO.: 14); the sequences of primer pair 7 are: forward primer 5′-AGATCTAAGGCCGGGAGAGG-3′ (SEQ ID NO:2), reverse primer 5′-CGCCCATTCGCCTCTAAAGT-3′ (SEQ ID NO: 3); the sequences of primer pair 10 are: forward primer 5′-AGGAAGCGGGTCTATGGTTG-3′ (SEQ ID NO:13), reverse primer 5′-GACTGAGAAGGTGGCCTAGC-3′ (SEQ ID NO:15). The results indicate that all three sets of primers can be used for the successful detection of BamHI-W.

### DETAILED DESCRIPTION OF THE PRESENT INVENTION

In light of the issues discussed in the background of the invention, the present disclosure provides a method of detecting and/or quantifying a microorganism that has better sensitivity and/or specificity compared to the currently available methods.

In one aspect, there is provided a method for detecting and/or quantifying the presence of a target nucleic acid sequence of a microorganism in a sample obtained from a subject, comprising amplifying the target sequence in a CpG island of the nucleic acid of the microorganism, irrespective of the methylation status of the CpG island.

The term “amplifying” or “amplification” as used herein refers to the production of additional copies of the target sequence.

The term “CpG island” as used herein refers to nucleic acid regions with a high frequency of CpG sites. A CpG site (or CG site) is a region of DNA where a cytosine nucleotide is followed by a guanine nucleotide in the linear sequence of nucleotides along its 5′ to 3′ direction. CpG is shorthand for 5′-C-phosphate-G-3′. A CpG island can have at least about 100 nucleotides, or at least about 500 nucleotides, or at least about 1000 nucleotides, or at least about 2000 nucleotides, or at least about 3000 nucleotides, or at least about 4000 nucleotides, or at least about 5000 nucleotides, or at least about 6000 nucleotides, or at least about 7000 nucleotides, or at least about 8000 nucleotides, or at least about 9000 nucleotides, or at least about 10000 nucleotides, or at least about 15000 nucleotides, or at least about 20000 nucleotides, or at least about 25000 nucleotides, or at least about 30000 nucleotides, or at least about 35000 nucleotides, or at least about 40000 nucleotides, or at least about 45000 nucleotides, or at least about 50000 nucleotides, or at least about 60000 nucleotides, or at least about 70000 nucleotides, or at least about 80000 nucleotides, or at least about 90000 nucleotides, or at least about 100000 nucleotides, or between about 100 nucleotides to about 5000 nucleotides, or between about 200 nucleotides to about 4900 nucleotides, or between about 300 nucleotides to about 4800 nucleotides, or between about 400 nucleotides to about 4700 nucleotides, or between about 500 nucleotides to about 4600 nucleotides, or between about 600 nucleotides to about 4500 nucleotides, or between about 700 nucleotides to about 4400 nucleotides, or between about 800 nucleotides to about 4300 nucleotides, or between about 900 nucleotides to about 4200 nucleotides, or between about 1000 nucleotides to about 4100 nucleotides, or between about 1100 nucleotides to about 4000 nucleotides, or between about 1200 nucleotides to about 3900 nucleotides, or between about 1300 nucleotides to about 3800 nucleotides, or between about 1400 nucleotides to about 3700 nucleotides, or between about 1500 nucleotides to about 3600 nucleotides, or between about 1600 nucleotides to about 3500 nucleotides, or between about 1700 nucleotides to about 3400 nucleotides, or between about 1800 nucleotides to about 3300 nucleotides, or between about 1900 nucleotides to about 3200 nucleotides, or between about 2000 nucleotides to about 3100 nucleotides, or between about 2100 nucleotides to about 3000 nucleotides, or between about 2200 nucleotides to about 2900 nucleotides, or between about 2300 nucleotides to about 2800 nucleotides, or between about 2400 nucleotides to about 2700 nucleotides, or between about 2500 nucleotides to about 2600 nucleotides, or about 150 nucleotides, or about 250 nucleotides, or about 350 nucleotides, or about 450 nucleotides, or about 550 nucleotides, or about 650 nucleotides, or about 750 nucleotides, or about 850 nucleotides, or about 950 nucleotides, or about 1050 nucleotides, or about 1150 nucleotides, or about 1250 nucleotides, or about 1350 nucleotides, or about 1450 nucleotides, or about 1550 nucleotides, or about 1650 nucleotides, or about 1750 nucleotides, or about 1850 nucleotides, or about 1950 nucleotides, or about 2050 nucleotides, or about 2150 nucleotides, or about 2250 nucleotides, or about 2350 nucleotides, or about 2450 nucleotides, or about 2550 nucleotides, or about 2650 nucleotides, or about 2750 nucleotides, or about 2850 nucleotides, or about 2950 nucleotides, or about 3050 nucleotides, or about 3150 nucleotides, or about 3250 nucleotides, or about 3350 nucleotides, or about 3450 nucleotides, or about 3550 nucleotides, or about 3650 nucleotides, or about 3750 nucleotides, or about 3850 nucleotides, or about 3950 nucleotides, or about 4050 nucleotides, or about 4150 nucleotides, or about 4250 nucleotides, or about 4350 nucleotides, or about 4450 nucleotides, or about 4550 nucleotides, or about 4650 nucleotides, or about 4750 nucleotides, or about 4850 nucleotides, or about 4950 nucleotides. A CpG island usually has a C, G percentage of greater than about 50%, or greater than about 51%, or greater than about 52%, or greater than about 53%, or greater than about 54%, or greater than about 55%, or greater than about 56%, or greater than about 57%, or greater than about 58%, or greater than about 59%, or greater than about 60%, or greater than about 61%, or greater than about 62%, or greater than about 63%, or greater than about 64%, or greater than about 65%, or greater than about 66%, or greater than about 67%, or greater than about 68%, or greater than about 69%, or greater than about 70%, or greater than about 71%, or greater than about 72%, or greater than about 73%, or greater than about 74%, or greater than about 75%, or greater than about 76%, or greater than about 77%, or greater than about 78%, or greater than about 79%, or greater than about 80%. The observed-to-expected CpG ratio in CpG island is usually greater than about 55%, or greater than about 56%, or greater than about 57%, or greater than about 58%, or greater than about 59%, or greater than about 60%, or greater than about 61%, or greater than about 62%, or greater than about 63%, or greater than about 64%, or greater than about 65%, or greater than about 66%, or greater than about 67%, or greater than about 68%, or greater than about 69%, or greater than about 70%, or greater than about 71%, or greater than about 72%, or greater than about 73%, or greater than about 74%, or greater than about 75%, or greater than about 76%, or greater than about 77%, or greater than about 78%, or greater than about 79%, or greater than about 80%, or greater than about 81%, or greater than about 82%, or greater than about 83%, or greater than about 84%, or greater than about 85%, or greater than about 86%, or greater than about 87%, or greater than about 88%, or greater than about 89%, or greater than about 90%, or greater than about 91%, or greater than about 92%, or greater than about 93%, or greater than about 94%, or greater than about 95%, or greater than about 96%, or greater than about 97%, or greater than about 98%, or greater than about 99%. The “observed-to-expected CpG ratio” can be derived where the “observed” is the actual number of CpGs in the sequence, and where the “expected” is calculated as:
(actual number of C×actual number of G)/length of sequence or as:
((actual number of C+actual number of G)/2)²/length of sequence.

A number of softwares or analytical tools can be used for the prediction of CpG island in a nucleic acid sequence. Examples of analytical tools available online include but are not limited to: Sequence Manipulation Suite available at http://www.bioinformatics.org/sms2/cpg_islands.html; and Emboss Cpgplot available at http://www.ebi.ac.uk/Tools/segstats/emboss_cpgplot/.

The term “target sequence” or “target nucleic acid sequence” or their grammatical variants as used herein refer to a region of the nucleic acid sequence of the microorganism of interest to be amplified. The presence of the target sequence in a sample obtained from a subject indicates the presence of the nucleic acid sequence associated with the microorganism of interest, and/or the presence of the microorganism in the sample or in the subject.

In some examples, it is preferred that the target sequence to be amplified for the detection and/or quantification of the microorganism is located within the 5′end of the CpG island. The 5′ end location would allow for preferential preservation during exonuclease III degradation of the DNA. Thus, in some examples, the target sequence is located within the first 50%, or the first 49%, or the first 48%, or the first 47%, or the first 46%, or the first 45%, or the first 44%, or the first 43%, or the first 42%, or the first 41%, or the first 40%, or the first 39%, or the first 38%, or the first 37%, or the first 36%, or the first 35%, or the first 34%, or the first 33%, or the first 32%, or the first 31%, or the first 30%, or the first 29%, or the first 28%, or the first 27%, or the first 26%, or the first 25%, or the first 24%, or the first 23%, or the first 22%, or the first 21%, or the first 20%, or the first 19%, or the first 18%, or the first 17%, or the first 16%, or the first 15%, or the first 14%, or the first 13%, or the first 12%, or the first 11%, or the first 10%, or the first 9%, or the first 8%, or the first 7%, or the first 6%, or the first 5%, or the first 4%, or the first 3%, or the first 2%, or the first 1% nucleotides of the CpG island of the nucleic acid of the microorganism, counted from the 5′ end.

The term “methylation” as used herein refers to DNA methylation which typically occurs at a CpG site. Such methylation results in the conversion of the cytosine to 5-methylcytosine, and can by catalysed by the enzyme DNA methyltransferase. A CpG cite can be either methylated or unmethylated.

The method of for detecting and/or quantifying the presence of a target nucleic acid sequence of a microorganism as provided in the present disclosure comprises amplifying the target sequence in a CpG island of the nucleic acid of the microorganism, irrespective of the methylation status of the CpG island. This means that the methylation status of the CpG island for which the target sequence lies within is not important. One of the advantages provided by such a method is that no methylation-status-specific sequencing will be required to select the target sequence to be amplified.

The term “microorganism” as used herein refers to a microscopic living organism, which may be single-celled or multicellular. The microorganism contains DNAs, thus microorganisms that do not contain DNAs (e.g. RNA viruses) are excluded. Examples of microorganisms include but are not limited to bacteria, DNA viruses, fungi and parasites. In some specific examples, the microorganisms are bacteria and DNA viruses. DNA viruses include but are not limited to DNA viruses with double stranded DNAs and DNA viruses with single stranded DNAs. In some examples, the microorganisms are pathogenic.

The term “pathogenic”, “pathogen” and other grammatical variants as used herein refer to the ability of the microorganisms to cause diseases. Examples of such diseases include but are not limited to acinetobacter infections, Actinomycosis, African sleeping sickness (African trypanosomiasis), AIDS (Acquired immunodeficiency syndrome), Amebiasis, Anaplasmosis, Angiostrongyliasis, Anisakiasis, Anthrax, Arcanobacterium haemolyticum infection, Argentine hemorrhagic fever, Ascariasis, Aspergillosis, Astrovirus infection, Alzheimer's disease, Amyotrophic lateral sclerosis, Anorexia nervosa, Anxiety disorder, Asthma, Atherosclerosis, Autoimmune diseases, Babesiosis, Bacillus cereus infection, Bacterial pneumonia, Bacterial vaginosis, Bacteroides infection, Balantidiasis, Bartonellosis, Baylisascaris infection, BK virus infection, Black piedra, Blastocystosis, Blastomycosis, Bolivian hemorrhagic fever, Botulism (and infant botulism), Brazilian hemorrhagic fever, Brucellosis, Bubonic plague, Burkholderia infection, Buruli ulcer, Calicivirus infection (norovirus and sapovirus), Campylobacteriosis, Candidiasis (moniliasis; thrush), Capillariasis, Carrion's disease, Cellulitis, Chagas disease, Chancroid, Chickenpox, Chikungunya, Chlamydia, Chlamydophila pneumoniae infection, Cholera, Chromoblastomycosis, Chytridiomycosis, Clonorchiasis, Clostridium difficile colitis, Coccidioidomycosis, Colorado tick fever, Common cold (acute viral rhinopharyngitis; acute coryza), Creutzfeldt-Jakob disease, Crimean-Congo hemorrhagic fever, Cryptococcosis, Cryptosporidiosis, Cutaneous larva migrans, Cyclosporiasis, Cysticercosis, Cytomegalovirus infection, Cancers, Chronic obstructive pulmonary disease, Crohn's disease, Coronary heart disease, Dengue fever, Desmodesmus infection, Dientamoebiasis, Diphtheria, Diphyllobothriasis, Dracunculiasis, Dementia, Diabetes mellitus type 1, Diabetes mellitus type 2, Dilated cardiomyopathy, Ebola hemorrhagic fever, Echinococcosis, Ehrlichiosis, Enterobiasis, Enterococcus infection, Enterovirus infection, Epidemic typhus, Erythema infectiosum, Exanthem subitum, Epilepsy, Epstein-Barr virus infectious mononucleosis, Fasciolasis, Fasciolopsiasis, Fatal familial insomnia, Filariasis, Food poisoning by clostridium perfringens, Free-living amebic infection, Fusobacterium infection, Gas gangrene (Clostridial myonecrosis), Geotrichosis, Gerstmann-Sträussler-Scheinker syndrome, Giardiasis, Glanders, Gnathostomiasis, Gonorrhoea, Granuloma inguinale, Group A Streptococcal infection, Group B Streptococcal infection, Guillain-Barré syndrome, Haemophilus influenzae infection, Hand, foot and mouth disease, Hantavirus pulmonary syndrome, Heartland virus disease, Helicobacter pylori infection, Hemolytic-uremic syndrome, Hemorrhagic fever with renal syndrome, Hepatitis A, Hepatitis B, Hepatitis C, Hepatitis D, Hepatitis E, Herpes simplex, Histoplasmosis, Hookworm infection, Human bocavirus infection, Human ewingii ehrlichiosis, Human granulocytic anaplasmosis, Human metapneumovirus infection, Human monocytic ehrlichiosis, Human papillomavirus infection, Human parainfluenza virus infection, Hymenolepiasis, Influenza, Isosporiasis, Irritable bowel syndrome, Kawasaki disease, Keratitis, Kingella kingae infection, Kuru, Lassa fever, Legionellosis, Legionellosis, Leishmaniasis, Leprosy, Leptospirosis, Listeriosis, Lyme disease, Lymphatic filariasis, Lymphocytic choriomeningitis, Lupus, Malaria, Marburg hemorrhagic fever, Measles, Middle East respiratory syndrome, Melioidosis, Meningitis, Meningococcal disease, Metagonimiasis, Microsporidiosis, Molluscum contagiosum, Monkeypox, mumps, Murine typhus, Mycoplasma pneumonia, Mycetoma, Myiasis, Multiple sclerosis, Myocardial infarction, Neonatal conjunctivitis, Nocardiosis, Onchocerciasis, Opisthorchiasis, Paracoccidioidomycosis, Paragonimiasis, Pasteurellosis, Pediculosis capitis, Pediculosis corporis, Pediculosis pubis, Pelvic inflammatory disease, Pertussis, Plague, Pneumococcal infection, Pneumocystis pneumonia, Panencephalitis, Pneumonia, Poliomyelitis, Prevotella infection, Primary amoebic meningoencephalitis, Progressive multifocal leukoencephalopathy, Psittacosis, Parkinson's disease, Psoriasis, Rabies, Relapsing fever, Respiratory syncytial virus infection, Rhinosporidiosis, Rhinovirus infection, Rickettsial infection, Rickettsialpox, Rift valley fever, Rocky mountain spotted fever, Rotavirus infection, Rubella, Rheumatoid arthritis, Salmonellosis, SARS, Scabies, Schistosomiasis, Sepsis, Shigellosis, Shingles, Smallpox (variola), Sporotrichosis, Staphylococcal food poisoning, Staphylococcal infection, Strongyloidiasis, Subacute sclerosing, Sarcoidosis, Schizophrenia, Stroke, Syphilis, Taeniasis, Tetanus, Tinea barbae, Tinea capitis, Tinea corporis, Tinea cruris, Tinea manum, Tinea nigra, Tinea pedis, Tinea unguium, Tinea versicolor, Toxocariasis, Toxocariasis, Trachoma, Toxoplasmosis, Trichinosis, Trichomoniasis, Trichuriasis, Tuberculosis, Tularemia, Typhoid fever, Typhus fever, Thromboangiitis obliterans, Tourette syndrome, Tuberculosis, Ureaplasma urealyticum infection, Vasculitis, Variant Creutzfeldt-Jakob disease, Valley fever, Venezuelan equine encephalitis, Venezuelan hemorrhagic fever, Vibrio vulnificus infection, Vibrio parahaemolyticus enteritis, Viral pneumonia, West nile fever, White piedra, Yersinia pseudotuberculosis infection, Yersiniosis, Yellow fever and Zygomycosis. In one specific example, the disease is cancer. In another specific example, the disease is tuberculosis.

One specific example of a DNA virus is Epstein-Barr virus (EBV), also called human herpesvirus 4 (HHV-4). EBV is one of the most common viruses in humans, and is commonly transmitted by saliva and established latent infection in B lymphocytes where it persists for the lifetime of the host. EBV infection is associated with particular forms of cancer, such as nasopharyngeal carcinoma, gastric cancer, Hodgkin's lymphoma and Burkitt's lymphoma.

In an example of the present disclosure, the target sequence to be amplified for the detection of EBV comprises a sequence within a CpG island of the BamHI-W region of EBV.

The term “BamHI-W region” as used herein refers to a repeating BamHI-W restriction fragment of the EBV genome. BamHI-W is a 3-kb long sequence and the genome of an EBV typically contains six to twenty copies of the BamHI-W sequence. The BamHI-W has the following sequence:

>J02072.1 Epstein Barr Virus large internal repeat
(BamHI-W fragment)
(SEQ ID NO.: 5)
GGATCCCCCCACCGGCCCTTCTCTCTGTCCCCCTGCTCCTCTCCAACCTT
CGCTCCACCCTAGACCCCAGCTTCTGGCCTCCCCGGGTCCACCAGGCCAG
CCGGAGGGACCCCGGCAGCCCGGGCGAGTCGCCTTCCCTCTCCCCTGGCC
TCTCCTTCCCGCCTCCCACCCGAGCCCCCTCAGCTTGCCTCCCCACCGGG
TCCATCAGGCCGGCCGGAGGGACCCCGGCGGCCCGGTGTCAGTCCCCCCT
GCAGCCGCCCAGTCTCTGCCTCCAGGCAAGGGCGCCAGCTTTTCTCCCCC
CAGCCTGAGGCCCAGTCTCCTGTGCACTGTCTGTAAAGTCCAGCCTCCCA
CGCCCGTCCACGGCTCCCGGGCCCAGCCTCGTCCACCCCTCCCCACGGTG
GACAGGCCCTCTGTCCACCCGGGCCATCCCCGCCCCCCTGTGTCCACCCC
AGTCCCGTCCAGGGGGGACTTTATGTGACCCTTGGGCCTGGCTCCCCATA
GACTCCCATGTAAGCCTGCCTCGAGTAGGTGCCTCCAGAGCCCCTTTTGC
CCCCCTGGCGGCCCAGCCCGACCCCCGGGCGCCCCCAAACTTTGTCCAGA
TGTCCAGGGGTCCCCGAGGGTGAGGCCCAGCCCCCTCCCGCCCCTGTCCA
CTGCCCCGGTCCCCCCAGAAGCCCCCAAAAGTAGAGGCTCAGGCCATGCG
CGCCCTGTCACCAGGCCTGCCAAAGAGCCAGATCTAAGGCCGGGAGAGGC
AGCCCCAAAGCGGGTGCAGTAACAGGTAATCTCTGGTAGTGATTTGGACC
CGAAATCTGACACTTTAGAGCTCTGGAGGACTTTAAAACTCTAAAAATCA
AAACTTTAGAGGCGAATGGGCGCCATTTTGTCCCCACGCGCGCATAATGG
CGGACCTAGGCCTAAAACCCCCAGGAAGCGGGTCTATGGTTGGCTGCGCT
GCTGCTATCTTTAGAGGGGAAAAGAGGAATAAGCCCCCAGACAGGGGAGT
GGGCTTGTTTGTGACTTCACCAAAGGTCAGGGCCCAAGGGGGTTCGCGTT
GCTAGGCCACCTTCTCAGTCCAGCGCGTTTACGTAAGCCAGACAGCAGCC
AATTGTCAGTTCTAGGGAGGGGGACCACTGCCCCTGGTATAAAGTGGTCC
TGCAGCTATTTCTGGTCGCATCAGAGCGCCAGGAGTCCACACAAATGTAA
GAGGGGGTCTTCTACCTCTCCCTAGCCCTCCGCCCCCTCCAAGGACTCGG
GCCCAGTTTCTAACTTTTCCCCTTCCCTCCCTCGTCTTGCCCTGCGCCCG
GGGCCACCTTCATCACCGTCGCTGACTCCGCCATCCAAGCCTAGGGGAGA
CCGAAGTGAAGGCCCTGGACCAACCCGGCCCGGGCCCCCCGGTATCGGGC
CAGAGGTAAGTGGACTTTAATTTTTTCTGCTAAGCCCAACACTCCACCAC
ACCCAGGCACACACTACACACACCCACCCGTCTCAGGGTCCCCTCGGACA
GCTCCTAAGAAGGCACCGGTCGCCCAGTCCTACCAGAGGGGGCCAAGAAC
CCAGACGAGTCCGTAGAAGGGTCCTCGTCCAGCAAGAAGAGGAGGTGGTA
AGCGGTTCACCTTCAGGGGTAAGTAACCTGACCTCTCCAGGGCTCACATA
AAGGGAGGCTTAGTATACATGCTTCTTGCTTTTCACAGGAACCTGGGGGC
TAGTCTGGGTGGGATTAGGCTGCCTCAAGTTGCATCAGCCAGGGCTTCAT
GCCCTCCTCAGTTCCCTAGTCCCCGGGCTTCAGGCCCCCTCCGTCCCCGT
CCTCCAGAGACCCGGGCTTCAGGCCCTGCCTCTCCTGTTACCCTTTTAGA
ACCACAGCCTGGACACATGTGCCAGACGCCTTGGCCTCTAAGGCCCTCGG
GTCCCCCTGGACCCCGGCCTCAGCAACCCTGCTGCTCCCCTCCTGCCACC
CCAGCCTCCCCCCCTCCCCGTCCCCCTTCGCTCCTGATCCTCCCCCGGTC
CCCAGTAGGGCCGCCTGCCCCCCTGCACCCAGTACCTGCCCCTCTTGGCC
ACGCACCCCGGGCCAGGCCACCTTAGACCCGGCCAAGCCCCATCCCTGAA
GACCCAGCGGCCATTCTCTCTGGTAACGAGCAGAGAAGAAGTAGAGGCCC
GCGGCCATTGGGCCCAGATTGAGAGACCAGTCCAGGGGCCCGAGGTTGGA
GCCAGCGGGCACCCGAGGTCCCAGCACCCGGTCCCTCCGGGGGGCAGAGA
CAGGCAGGGCCCCCCGGCAGCTGGCCCCGAGGAGGCGCCCGGAGTGGGGC
CGGTCGGCTGGGCTGGCCGAGCCCGGGTCTGGGAGGTCTGGGGTGGCGAG
CCTGCTGTCTCAGGAGGGGCCTGGCTCCGCCGGGTGGCCCTGGGGTAAGT
CTGGGAGGCAGAGGGTCGGCCTAGGCCCGGGGAAGTGGAGGGGGATCGCC
CGGGTCTCTGTTGGCAGAGTCCGGGCGATCCTCTGAGACCCTCCGGGCCC
GGACGGTCGCCCTCAGCCCCCCAGACAGACCCCAGGGTCTCCAGGCAGGG
TCCGGCATCTTCAGGGGCAGCAGGCTCACCACCACAGGCCCCCCAGACCC
GGGTCTCGGCCAGCCGAGCCGACCGGCCCCGCGCCTGGCGCCTCCTCGGG
GCCAGCCGCCGGGGTTGGTTCTGCCCCTCTCTCTGTCCTTCAGAGGAACC
AGGGACCTCGGGCACCCCAGAGCCCCTCGGGCCCGCCTCCAGGCGCCCTC
CTGGTCTCCGCTCCCCTCTGAGCCCCGTTAAACCCAAAGAATGTCTGAGG
GGAGCCACCCTCGGGGCCCAGGCCCCAGAGTCCAGAGGTCAGGGGCACCT
CAGGGTGCCTCCCCGGGTCCCAGGCCAGCCGGAGGGACCCCGGCAGCCCG
GGCGGCCCCAGAGGCCGGTTCCTCGCCCCTTCCCCGGGCTTCAGAGCCCA
GGATGTCCCCCAGAAGGGACCCTAGGCGTCCCCTCTCCTCCCCTCCAGGC
CCGAGCCTCTCCCTCGCGGAGAGGGGCCTCTTTGGGCCCTCAAGTCCAGC
CCCACCGAGACCCGAGTGGCCC.

There has been evidence showing EBV has evolved to utilize DNA methylation to maximize persistence and to protect itself from immune detection. Moreover, methylation of EBV BamHI-W fragment is important to its expression as a W promoter that drives expression of the EB viral nuclear antigens (EBNAs) at the initiation of virus-induced B-cell transformation. The role of methylation in EBV activity has also been recognized in human tissue. Pharmacologic reversal of dense CpG methylation in tumor tissue can be achieved in patients undergoing treatment with DNA methyltransferase inhibitor. On the other hand, the CpG island of EBV BamHI-W has been shown to be unmethylated in the latent cycle of EBV. Despite the variance in the methylation status of the target nucleic acid sequence within the CpG island of EBV, the method as disclosed herein can be used for the detection and/or quantification of such a target sequence.

In some examples, the CpG island of the BamHI-W region of EBV is selected from the group consisting of:

(SEQ ID NO.: 6)
CTCCTCTCCAACCTTCGCTCCACCCTAGACCCCAGCTTCTGGCCTCCCCG
GGTCCACCAGGCCAGCCGGAGGGACCCCGGCAGCCCGGGCGAGTCGCCTT
CCCTCTCCCCTGGCCTCTCCTTCCCGCCTCCCACCCGAGCCCCCTCAGCT
TGCCTCCCCACCGGGTCCATCAGGCCGGCCGGAGGGACCCCGGCGGCCCG
GTGTCA (nucleotides at position number 36 to 241
of BamHI-W region),
(SEQ ID NO.: 7)
AGGCCATGCGCGCCCTGTCACCAGGCCTGCCAAAGAGCCAGATCTAAGGC
CGGGAGAGGCAGCCCCAAAGCGGGTGCAGTAACAGGTAATCTCTGGTAGT
GATTTGGACCCGAAATCTGACACTTTAGAGCTCTGGAGGACTTTAAAACT
CTAAAAATCAAAACTTTAGAGGCGAATGGGCGCCATTTTGTCCCCACGCG
CGCATAATGGCGGACCTAGGCCTAAAACCCCCAGGAAGCGGGTCTATGGT
TGGCTGCGCTGCTGCTATCTTTAGAGGGGAAAAGAGGAATAAGCCCCCAG
ACAGGGGAGTGGGCTTGTTTGTGACTTCACCAAAGGTCAGGGCCCAAGGG
GGTTCGCGTTGCTAGGCCACCTTCTCAGTC CAGCGCGTTTACGTAAGC
(nucleotides at position number 691 to 1088 of
BamHI-W region),
(SEQ ID NO.: 8)
CTGGTATAAAGTGGTCCTGCAGCTATTTCTGGTCGCATCAGAGCGCCAGG
AGTCCACACAAATGTAAGAGGGGGTCTTCTACCTCTCCCTAGCCCTCCGC
CCCCTCCAAGGACTCGGGCCCAGTTTCTAACTTTTCCCCTTCCCTCCCTC
GTCTTGCCCTGCGCCCGGGGCCACCTTCATCACCGTCGCTGACTCCGCCA
TCCAAGCCTAGGGGAGACCGAAGTGAAGGCCCTGGACCAACCCGGCCCGG
GCCCCCCGGTATCGGGCCAGAGGTAAGTGGACTTTAATTTTTTCTGCTAA
GCCCAACACTCCACCACACCCAGGCACACACTACACACACCCACCCGTCT
CAGGGTCCCCTCGGA (nucleotides at position number
1134 to 1498 of BamHI-W region),
and
(SEQ ID NO.: 9)
CGAGGAGGCGCCCGGAGTGGGGCCGGTCGGCTGGGCTGGCCGAGCCCGGG
TCTGGGAGGTCTGGGGTGGCGAGCCTGCTGTCTCAGGAGGGGCCTGGCTC
CGCCGGGTGGCCCTGGGGTAAGTCTGGGAGGCAGAGGGTCGGCCTAGGCC
CGGGGAAGTGGAGGGGGATCGCCCGGGTCTCTGTTGGCAGAGTCCGGGCG
ATCCTCTGAGACCCTCCGGGCCCGGACGGTCGCCCTCAGCCCCCCAGACA
GACCCCAGGGTCTCCAGGCAGGGTCCGGCATCTTCAGGGGCAGCAGGCTC
ACCACCACAGGCCCCCCAGACCCGGGTCTCGGCCAGCCGAGCCGACCGGC
CCCGCGCCTGGCGCCTCCTCGGGGCCAGCCGCCGGGGTTGGTTCTGCCCC
TCTCTCTGTCCTTCAGAGGAACCAGGGACCTCG
GGCACCCCAGAGCCCCTCGGGCCCGCCTCCAGGCGCCCTCCTGGTCTCCG
CTCCCCTCTGAGCCCCGTTAAACCCAAAGAATGTCTGAGGGGAGCCACCC
TCGG (nucleotides at position number 2278 to 2814
of BamHI-W region).

In some examples, the target sequence within the CpG island comprises the sequence selected from the group consisting of:

(SEQ ID NO.: 1)
AGATCTAAGGCCGGGAGAGGCAGCCCCAAAGCGGGTGCAGTAACAGGTAA
TCTCTGGTAGTGATTTGGACCCGAAATCTGACACTTTAGAGCTCTGGAGG
ACTTTAAAACTCTAAAAATCAAAACTTTAGAGGCGAATGGGCG
(nucleotides at position number 730 to 872 of
BamHI-W region),
(SEQ ID NO.: 10)
GGAATAAGCCCCCAGACAGGGGAGTGGGCTTGTTTGTGACTTCACCAAAG
GTCAGGGCCCAAGGGGGTTCGCGTTGCTAGGCCACCTTCTCAGTCCAGCG
CGTTTACGTAA (nucleotides at position number 976
to 1086 of BamHI-W region),
(SEQ ID NO.: 11)
AGGAAGCGGGTCTATGGTTGGCTGCGCTGCTGCTATCTTTAGAGGGGAAA
AGAGGAATAAGCCCCCAGACAGGGGAGTGGGCTTGTTTGTGACTTCACCA
AAGGTCAGGGCCCAAGGGGGTTCGCGTTGCTAGGCCACCTTCTCAGTC
(nucleotides at position number 923 to 1070 of
BamHI-W region), a complementary sequence, a
fragment and a variant thereof.

The term “fragment” as used herein refers to a nucleic acid sequence that is a constituent of the reference, or a constituent of the complementary sequence of the reference sequence. In some examples, a fragment comprises about 9 to about 300 nucleotides, or about 10 to about 290 nucleotides, or about 20 to about 280 nucleotides, or about 30 to about 270 nucleotides, or about 40 to about 260 nucleotides, or about 50 to about 250 nucleotides, or about 60 to about 240 nucleotides, or about 70 to about 230 nucleotides, or about 80 to about 220 nucleotides, or about 90 to about 210 nucleotides, or about 100 to about 200 nucleotides, or about 110 to about 190 nucleotides, or about 120 to about 180 nucleotides, or about 130 to about 170 nucleotides, or about 140 to about 160 nucleotides, or 10 nucleotides, 15 nucleotides, 25 nucleotides, or 35 nucleotides, or 45 nucleotides, or 55 nucleotides, or 65 nucleotides, or 75 nucleotides, or 85 nucleotides, or 95 nucleotides, or 105 nucleotides, or 115 nucleotides, or 125 nucleotides, or 135 nucleotides, or 145 nucleotides, or 155 nucleotides, or 165 nucleotides, or 175 nucleotides, or 185 nucleotides, or 195 nucleotides, or 205 nucleotides, or 215 nucleotides, or 225 nucleotides, or 235 nucleotides, or 245 nucleotides, or 255 nucleotides, or 265 nucleotides, or 275 nucleotides, or 285 nucleotides, or 295 nucleotides of the reference sequence or the complementary sequence of the reference sequence. A fragment can also comprise about 5% to about 95%, about 10% to about 90%, about 15% to about 85%, about 20% to about 80%, about 25% to about 75%, about 30% to about 70%, about 35% to about 65%, about 40% to about 60%, about 45% to about 55%, or about 50% of the reference sequence or the complementary sequence of the reference sequence.

The term “variant” as used herein refers to sequences that are substantially similar to the reference sequence. These nucleotide sequence variants may have at least 25%, 30%, 35%, 40%, 45%, 50%, 55%, 60%, 65%, 70%, 75%, 80%, 85%, 90%, 95%, 96%, 97%, 98% or 99% sequence identity to the “non-variant” reference sequence. Variants may be a result of substitution, deletion or additional of any number of nucleotides in the reference sequence as a result of the mutation of the wild type DNA.

In one example, the amplification of the target sequence comprises the use of at least one oligonucleotide primer capable of binding to the target sequence. The at least one oligonucleotide primer binds within a region from about 9 to about 300 nucleotides, or about 10 to about 290 nucleotides, or about 20 to about 280 nucleotides, or about 30 to about 270 nucleotides, or about 40 to about 260 nucleotides, or about 50 to about 250 nucleotides, or about 60 to about 240 nucleotides, or about 70 to about 230 nucleotides, or about 80 to about 220 nucleotides, or about 90 to about 210 nucleotides, or about 100 to about 200 nucleotides, or about 110 to about 190 nucleotides, or about 120 to about 180 nucleotides, or about 130 to about 170 nucleotides, or about 140 to about 160 nucleotides, from the first nucleotide of the target sequence. The length of the at least one primer is between 5 to 40 nucleotides, or between 10 to 35 nucleotides, or between 15 to 30 nucleotides, or between 20 to 25 nucleotides, or 8 nucleotides, or 9 nucleotides, or 10 nucleotides, or 12 nucleotides, or 14 nucleotides, or 16 nucleotides, or 18 nucleotides, or 20 nucleotides, or 22 nucleotides, or 24 nucleotides, or 26 nucleotides, or 28 nucleotides, or 30 nucleotides, or 32 nucleotides, or 34 nucleotides, or 36 nucleotides, or 38 nucleotides, or 40 nucleotides. In one specific example, the length of the at least one primer is 20 nucleotides.

In one example, the at least one primer has a sequence identity of at least 70%, at least 75%, at least 80%, at least 85%, at least 90%, at least 95%, at least 96%, at least 97%, at least 98% or at least 99% to at least one sequence selected from the group consisting of AGATCTAAGGCCGGGAGAGG (SEQ ID NO.: 2), GGAATAAGCCCCCAGACAGG (SEQ ID NO.: 12), AGGAAGCGGGTCTATGGTTG (SEQ ID NO.: 13), CGCCCATTCGCCTCTAAAGT (SEQ ID NO.: 3), TTACGTAAACGCGCTGGACT (SEQ ID NO.: 14), and GACTGAGAAGGTGGCCTAGC (SEQ ID NO.: 15), or a complementary sequence thereof. In one example, the primer comprises one sequence selected from the group consisting of AGATCTAAGGCCGGGAGAGG (SEQ ID NO.: 2), GGAATAAGCCCCCAGACAGG (SEQ ID NO.: 12), AGGAAGCGGGTCTATGGTTG (SEQ ID NO.: 13), CGCCCATTCGCCTCTAAAGT (SEQ ID NO.: 3), TTACGTAAACGCGCTGGACT (SEQ ID NO.: 14), and GACTGAGAAGGTGGCCTAGC (SEQ ID NO.: 15), or a complementary sequence thereof.

In another example, the amplification of the target sequence comprises the use of at least one pair of oligonucleotide primers. The at least one pair of oligonucleotide primers can comprise one forward primer and one reverse primer. In some examples, the at least one pair of oligonucleotide primers can be selected from the group consisting of: forward primer AGATCTAAGGCCGGGAGAGG (SEQ ID NO.: 2) and reverse primer CGCCCATTCGCCTCTAAAGT (SEQ ID NO.: 3); forward primer GGAATAAGCCCCCAGACAGG (SEQ ID NO.: 12) and reverse primer TTACGTAAACGCGCTGGACT (SEQ ID NO.: 14); forward primer AGGAAGCGGGTCTATGGTTG (SEQ ID NO.: 13) and reverse primer GACTGAGAAGGTGGCCTAGC (SEQ ID NO.: 15). In one specific example, the at least one pair of oligonucleotide primers are forward primer AGATCTAAGGCCGGGAGAGG (SEQ ID NO.: 2) and reverse primer CGCCCATTCGCCTCTAAAGT (SEQ ID NO.: 3).

In one example, the amplification of the target sequence comprises the use of a probe capable of binding to the target sequence. The probe binds within a region from between 9 to 500 nucleotides, or from between 10 to 490 nucleotides, or from between 20 to 480 nucleotides, or from between 30 to 470 nucleotides, or from between 40 to 460 nucleotides, or from between 50 to 450 nucleotides, or from between 60 to 440 nucleotides, or from between 70 to 430 nucleotides, or from between 80 to 420 nucleotides, or from between 90 to 410 nucleotides, or from between 100 to 400 nucleotides, or from between 110 to 390 nucleotides, or from between 120 to 380 nucleotides, or from between 130 to 370 nucleotides, or from between 140 to 360 nucleotides, or from between 150 to 350 nucleotides, or from between 160 to 340 nucleotides, or from between 170 to 330 nucleotides, or from between 180 to 320 nucleotides, or from between 190 to 310 nucleotides, or from between 200 to 300 nucleotides, or from between 210 to 290 nucleotides, or from between 220 to 280 nucleotides, or from between 230 to 270 nucleotides, or from between 240 to 260 nucleotides, or 8 nucleotides, 15 nucleotides, 25 nucleotides, or 35 nucleotides, or 45 nucleotides, or 55 nucleotides, or 65 nucleotides, or 80 nucleotides, or 90 nucleotides, or 100 nucleotides, or 110 nucleotides, or 120 nucleotides, or 130 nucleotides, or 140 nucleotides, or 150 nucleotides, or 160 nucleotides, or 170 nucleotides, or 180 nucleotides, or 190 nucleotides, or 200 nucleotides, or 210 nucleotides, or 220 nucleotides, or 230 nucleotides, or 240 nucleotides, or 250 nucleotides, or 260 nucleotides, or 270 nucleotides, or 280 nucleotides, or 290 nucleotides, or 300 nucleotides, or 310 nucleotides, or 320 nucleotides, or 330 nucleotides, or 340 nucleotides, or 350 nucleotides, or 360 nucleotides, or 370 nucleotides, or 380 nucleotides, or 390 nucleotides, or 400 nucleotides, or 410 nucleotides, or 420 nucleotides, or 430 nucleotides, or 440 nucleotides, or 450 nucleotides, or 460 nucleotides, or 470 nucleotides, or 480 nucleotides, or 490 nucleotides, or 500 nucleotides from a specific nucleotide of the target sequence. The specific nucleotide can be nucleotide at position number 1, 5, 10, 15, 20, 25, 30, 35, 40, 45, 50, 52, 54, 56, 58, 60, 62, 64, 66, 68, 70, 72, 74, 76, 78, 80, 82, 84, 86, 88, 90, 92, 94, 96, 98, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500 of the target sequence. The length of a probe can be between 5 to 40 nucleotides, or between 10 to 35 nucleotides, or between 15 to 30 nucleotides, or between 20 to 25 nucleotides, or 8 nucleotides, or 9 nucleotides, or 10 nucleotides, or 12 nucleotides, or 14 nucleotides, or 16 nucleotides, or 18 nucleotides, or 20 nucleotides, or 22 nucleotides, or 24 nucleotides, or 26 nucleotides, or 28 nucleotides, or 30 nucleotides, or 32 nucleotides, or 34 nucleotides, or 36 nucleotides, or 38 nucleotides, or 40 nucleotides.

In one example, the probe has a sequence identity of at least 70%, at least 75%, at least 80%, at least 85%, at least 90%, at least 95%, at least 96%, at least 97%, at least 98% or at least 99% to a sequence selected from the group consisting of CTCTGGTAGTGATTTGGACCCGAAATCTG (SEQ ID NO.: 16), CCACCTTCTCAGTCCAGCGCGTTT (SEQ ID NO.: 17), GTGACTTCACCAAAGGTCAGGGCCC (SEQ ID NO.: 18), GGTGGTAAGCGGTTCACCTTCAGGG (SEQ ID NO.: 19), and complementary sequences thereof. In one example, the probe comprises the sequence selected from the group consisting of CTCTGGTAGTGATTTGGACCCGAAATCTG (SEQ ID NO.: 16), CCACCTTCTCAGTCCAGCGCGTTT (SEQ ID NO.: 17), GTGACTTCACCAAAGGTCAGGGCCC (SEQ ID NO.: 18), GGTGGTAAGCGGTTCACCTTCAGGG (SEQ ID NO.: 19) and complementary sequences thereof. In one specific example, the probe comprises the sequence of CTCTGGTAGTGATTTGGACCCGAAATCTG (SEQ ID NO.: 16).

In one example, the amplification of the target sequence comprises the use of at least one oligonucleotide primer and one probe as defined above. In another example, the amplification of the target sequence comprises the use of at least one pair of oligonucleotide primers and one probe as defined above.

In one aspect, there is provided a method for detecting and/or quantifying the presence of a target nucleic acid sequence of Epstein-Barr virus (EBV) in a sample obtained from a subject, comprising amplifying a target sequence in the BamHI-W region of EBV, wherein the target sequence comprises the sequence of AGATCTAAGGCCGGGAGAGGCAGCCCCAAAGCGGGTGCAGTAACAGGTAATC TCTGGTAGTGATTTGGACCCGAAATCTGACACTTTAGAGCTCTGGAGGACTTTA AAACTCTAAAAATCAAAACTTTAGAGGCGAATGGGCG (SEQ ID NO.: 1), wherein amplifying the target sequence comprises the use of a pair of oligonucleotide primers and a probe, wherein the first oligonucleotide primer comprises the sequence of 5′-AGATCTAAGGCCGGGAGAGG-3′ (SEQ ID NO.: 2), and the second oligonucleotide primer comprises the sequence of 5′-CGCCCATTCGCCTCTAAAGT-3′ (SEQ ID NO.: 3), and wherein the probe comprises the sequence of 5′-(6-FAM)CTCTGGTAGTGATTTGGACCCGAAATCTG(TAMRA)-3′ (SEQ ID NO.: 4), and wherein the method is a quantitative polymerase chain reaction (qPCR).

In some examples, the method of detecting and/or quantifying the presence of a target nucleic acid sequence of Epstein-Barr virus (EBV) in a sample obtained from a subject further comprises amplifying a control.

The term “control” or “internal control” as used herein refers to a reference sequence which can be used to indicate whether the amplification system is functioning.

When a control is included in the amplification system but is not successfully amplified, it indicates that the amplification result is false-negative. When there is no control spiked in samples, it is not possible to differentiate true negative and false negative. When a control is spiked and successfully amplified but no target signal in the tested samples, the results are true negative. When internal control is spiked and no signals from both internal control and tested samples, the results are false negative. Thus using a control allows one to distinguish false negative results which are common in clinical samples in which many PCR inhibitors are present. False negative results have great impacts on patients' lives, such as failure of disease diagnosis which will delay treatment and might result in treatment failure.

In some examples, the control includes a target sequence of

(SEQ ID NO.: 80)
TTAGCAGCGACGAAGATCATGCGCTCACGCTCTCGGTGTCCTCATTCATC
AGTTATTCACAACGCTATGCTGTAACTCGACCTGACAAGACTGTACCTAT
GAGAAGGCACTTGCTACCTTATGCAAGCGTCAGCCCGCGGTATCGCTTG
G,

a complementary sequence, a fragment or a variant thereof.

In some examples, amplifying the control includes the use of a pair of oligonucleotide primers. Examples of a forward primer sequence is 5′-CGCTCTCGGTGTCCTCATTC-3′ (SEQ ID NO.: 81), a complementary sequence, a fragment or a variant thereof. Examples of a reverse primer sequence is 5′-GGCTGACGCTTGCATAAGGT-3′ (SEQ ID NO.: 82), a complementary sequence, a fragment or a variant thereof.

In some other examples, amplifying the control further includes the use of a probe capable of binding to the target sequence of the control. In one example, the probe comprises the sequence of CACAACGCTATGCTGTAACTCGACCTGAC (SEQ ID NO.: 89), a complementary sequence, a fragment or a variant thereof. One specific example of a probe is 5′-VIC-CACAACGCTATGCTGTAACTCGACCTGAC-TAMRA-3′ (SEQ ID NO.: 83).

In one example, the microorganism of which a target nucleic acid sequence is to be detected and/or quantified using the method as disclosed herein is a bacterium. One specific example of such a bacterium is Mycobacterium tuberculosis.

The following are some non-exclusive examples of CpG islands in the genomic nucleic acid sequences of Mycobacterium tuberculosis as predicted using CpG island analytical software:

| CpG island | Start position | End position |
|---|---|---|
| serial no. | (nucleotide number) | (nucleotide number) |
| 1 | 250 | 485,607 |
| 2 | 486,395 | 598,721 |
| 3 | 598,929 | 889,723 |
| 4 | 890,230 | 1,103,130 |
| 5 | 1,103,439 | 1,691,493 |
| 6 | 1,691,745 | 1,697,185 |
| 7 | 1,697,464 | 1,779,283 |
| 8 | 1,779,789 | 2,268,374 |
| 9 | 2,268,597 | 2,367,062 |
| 10 | 2,367,073 | 2,680,742 |
| 11 | 2,681,249 | 2,807,592 |
| 12 | 2,807,742 | 3,587,732 |
| 13 | 3,588,238 | 3,791,614 |
| 14 | 3,791,647 | 3,792,550 |
| 15 | 3,793,135 | 3,798,679 |
| 16 | 3,798,717 | 3,964,564 |
| 17 | 3,965,008 | 4,411,274 |

The above identified 17 CpG islands accounts for 99.9% of the genomic nucleic acid sequence of Mycobacterium tuberculosis.

Some non-limiting examples of the target sequences to be used for the detection and/or quantification of Mycobacterium tuberculosis are:

>ST-E00142:243:HVLMVCCXX:8:1101:23043:33111/1
(SEQ ID NO.: 20)
GGTCGCCGCGGGCAGGCTCAACCCGGAGCGGATCACCGAATCCACGATCG
CCCGCCACCTGCAGCGACCCGACATTCCCGACGTTGACCTCTTCCTGCGG
ACCTCGGGTGAGCAGCGCTCCAGCAACTTCATGCTGTGGCAGGCGGCCTA
(Nucleotide 2,642,836-2,642,687)
>ST-E00142:243:HVLMVCCXX:8:1101:23043:33111/2
(SEQ ID NO.: 21)
CCTCGCAGGCCGCCCACAAGTCGCGGCGGTCATAGTCGGGCCAGAGCTTG
TCCTGGAATATGTATTCAGCGTAGGCCGCCTGCCACAGCATGAAGTTGCT
GGAGCGCTGCTCACCCGAGGTCCGCAGGAAGAGGTCAACGTCGGGAATGT
(Nucleotide 2,642,616-2,642,765)
>ST-E00142:243:HVLMVCCXX:8:1101:24068:32601/1
(SEQ ID NO.: 22)
GGTCGCCGCGGGCAGGCTCAACCCGGAGCGGATCACCGAATCCACGATCG
CCCGCCACCTGCAGCGACCCGACATTCCCGACGTTGACCTCTTCCTGCGG
ACCTCGGGTGAGCAGCGCTCCAGCAACTTCATGCTGTGGCAGGCGGCCTA
(Nucleotide 2,642,836-2,642,687)
>ST-E00142:243:HVLMVCCXX:8:1101:24068:32601/2
(SEQ ID NO.: 23)
CCTCGCAGGCCGCCCACAAGTCGCGGCGGTCATAGTCGGGCCAGAGCTTG
TCCTGGAATATGTATTCAGCGTAGGCCGCCTGCCACAGCATGAAGTTGCT
GGAGCGCTGCTCACCCGAGGTCCGCAGGAAGAGGTCAACGTCGGGAATGT
(Nucleotide 2,642,616-2,642,765)
>ST-E00142:243:HVLMVCCXX:8:1103:21836:42622/1
(SEQ ID NO.: 24)
TGGTGGTCGGCTCGCGGCCGGTGTCGAGATCACCGAACCGCAAAGAAGTG
TGGTCGTCTTCGACACCGCACCCACCGCCCTGCTGCGGGTTTACCGCGAC
AAGCTTCC (Nucleotide 3,355,782-3,355,889)
>ST-E00142:243:HVLMVCCXX:8:1103:21836:42622/2
(SEQ ID NO.: 25)
GGAAGCTTGTCGCGGTAAACCCGCAGCAGGGCGGTGGGTGCGGTGTCGAA
GACGACCACACTTCTTTGCGGTTCGGTGATCTCGACACCGGCCGCGAGCC
GACCACCA (Nucleotide 3,355,889-3,355,782)
>ST-E00142:243:HVLMVCCXX:8:1111:4980:34799/1
(SEQ ID NO.: 26)
GATGTCCGGGCTCACCCCGGCGTGGTCGGCGGCGAACAACGCGCCGGTGC
GGCCGAAGCCGGTGGCGATCTCATCGAAGATCAGCAGCACCTCGTAACGG
CGGCAGATGTCCCGCAGGTCGTGCAGATAGCGCGGGTCGTGAAAACGCAT
(Nucleotide 1,776,225-1,776,076)
>ST-E00142:243:HVLMVCCXX:8:1111:4980:34799/2
(SEQ ID NO.: 27)
GCACGCCGGCGAGCTGGCCGCGGTGGTCGTGGAGCCGGTCGTGCAGGGTG
CGGGCGGTATGCGTTTTCACGACCCGCGCTATCTGCACGACCTGCGGGAC
ATCTGCCGCCGTTACGAGGTGCTGCTGATCTTCGATGAGATCGCCACCGG
(Nucleotide 1,776,018-1,776,167)
>ST-E00142:243:HVLMVCCXX:8:1203:1895:39335/1
(SEQ ID NO.: 28)
GGTATGGTCGCCGCCGCGACGCCGCTGCCGGTGATCGGGGTGCCCGTACC
GCTGGGCAGGCTGGACGGGCTTGACTCCCTGCTGTCGATCGTGCAAACGC
CGGCCGGGGGTCCGGTGGCCACGCTCCCCATCGGGGGCGCCGGTAAGGCC
(Nucleotide 3,658,407-3,658,258)
>ST-E00142:243:HVLMVCCXX:8:1203:1895:39335/2
(SEQ ID NO.: 29)
CCTCGGGCAGCTCGAACAGATCGAACGACGGGTTTCCGGCCCATCCGACC
ATCTTGGAGCCCTCCTAATCTCCGGGCTAGTCGCGGGTTAACTTACCCGG
CGGCCGCTCCACTTCCGCATCCTTGGCCGCCACGCCGTCGGCGAGCCGGT
(Nucleotide 3658038-3658187)
>ST-E00142:243:HVLMVCCXX:8:1203:25976:49127/1
(SEQ ID NO.: 30)
GTCATGCTGATCGCGGTGCTCGCTCGGCTGATGATGCGCGGCTGGCGGCG
CCGTTCG (Nucleotide 1555525-1555581)
>ST-E00142:243:HVLMVCCXX:8:1203:25976:49127/2
(SEQ ID NO.: 31)
CGAACGGCGCCGCCAGCCGCGCATCATCAGCCGAGCGAGCACCGCGATCA
GCATGAC (Nucleotide 1555581-1555525)
>ST-E00142:243:HVLMVCCXX:8:1206:20740:34570/1
(SEQ ID NO.: 32)
TGCATGGCTGTCGTCAGCTCGTGTCGTGAGATGTTGGGTTAAGTCCCGCA
ACGAGCGCAACCCTTGTCATTAGTTGC (Nucleotide 1472888-
1472955)
>ST-E00142:243:HVLMVCCXX:8:1207:9587:30334/1
(SEQ ID NO.: 33)
CCGGCTGGCGCAGCCGATACACCTCGCGCCACATCATGCCGGCCTCGAAG
TCGGCTCCGGCGAACGACTTT (Nucleotide 4010375-4010445)
>ST-E00142:243:HVLMVCCXX:8:1207:9587:30334/2
(SEQ ID NO.: 34)
AAAGTCGTTCGCCGGAGCCGACTTCGAGGCCGGCATGATGTGGCGCGAGG
TGTATCGGCTGCGCCAGCCGG (Nucleotide 4010445-4010375)
>ST-E00142:243:HVLMVCCXX:8:1211:11373:3366/1
(SEQ ID NO.: 35)
ACCCGCGGATACGGGTCCGGTACCGCCAACCGCGGCGCCGACGGCCGCCG
GCGCCGCGGCGGCAGCTTCGGCGGCCTCGGGACCGATCCATCCCAGCGCC
CGCCACGATGTAATAAGGCTGTTGCCAATACCAATGGCGAAATATGGCAA
(Nucleotide 1931752-1931901)
>ST-E00142:243:HVLMVCCXX:8:1211:11373:3366/2
(SEQ ID NO.: 36)
CCGGGGTGGCTGGAATGGTTCATCAACTGGTATCTGCCGATATCACAGCT
GTTTTACAACACCGTGGGTTTGCCATATTTCGCCATTGGTATTGGCAACA
GCCTTATTACATCGTGGCGGGCGCTGGGATGGATCGGTCCCGAGGCCGCC
(Nucleotide 1931970-1931821)
>ST-E00142:243:HVLMVCCXX:8:1211:29498:4280/1
(SEQ ID NO.: 37)
CAGGTAGCCGGTCAGCCGGATTGGGTCTCGTTGCGCCGGCAGGTGACGGT
CGCGCAGCGAAAAAGCGACCTGCGGGCCGCCGAGGATCCGATCGACGCCG
TCGTATGCGCC (Nucleotide 869238-869128)
>ST-E00142:243:HVLMVCCXX:8:1211:29498:4280/2
(SEQ ID NO.: 38)
GGCGCATACGACGGCGTCGATCGGATCCTCGGCGGCCCGCAGGTCGCTTT
TTCGCTGCGCGACCGTCACCTGCCGGCGCAACGAGACCCAATCCGGCTGA
CCGGCTACCTG (Nucleotide 869128-869238)
>ST-E00142:243:HVLMVCCXX:8:1211:3518:26255/1
(SEQ ID NO.: 39)
TCACATCCGGGTGGCGGGCAGCCGTCGTCTGCAACGAGGCCGACGGTAGG
CGATGGGTGAGGCCATGGAGGGCAAATGTCCCCATGGTGTATCGATTACG
TACCCTCTTACTTCGTCGGGGTCGCATCGGCATTCGCCTTGCCCGCCTGT
(Nucleotide 1934277-1934128)
>ST-E00142:243:HVLMVCCXX:8:1211:3518:26255/2
(SEQ ID NO.: 40)
CGGGCGACACACCGTCCCGATACATGTCAGCAACCGGGTCGATCGTGGTG
AATGCACAGGCGGGCAAGGCGAATGCCGATGCGACCCCGACGAAGTAAGA
GGGTACGTAATCGATACACCATGGGGACATTTGCCCTCCATGGCCTCACC
(Nucleotide 2023240-2023389)
>ST-E00142:243:HVLMVCCXX:8:1214:4706:5950/1
(SEQ ID NO.: 41)
ACCGGTGTCTTCTGCCATCAACAGGCCTCGACCGCGGGGCAGCGGGCCGC
CCTTCATCTTGCCGCGAATGAAGCCCTCGTCGGGATCGGCGTCCATCACC
AGCAGTGGCGCATTGGCCTGATGCAGGGCCCGCAACATCGGGTCGCTGCC
(Nucleotide 2023393-2023244)
>ST-E00142:243:HVLMVCCXX:8:1214:4706:5950/2
(SEQ ID NO.: 42)
AGCCGGCAGCGACCCGATGTTGCGGGCCCTGCATCAGGCCAATGCGCCAC
TGCTGGTGATGGACGCCGATCCCGACGAGGGCTTCATTCGCGGCAAGATG
AAGGGCGGCCCGCTGCCCCGCGGTCGAGGCCTGTTGATGGCAGAAGACAC
(Nucleotide 2023240-2023389)
>ST-E00142:243:HVLMVCCXX:8:1215:29812:28664/1
(SEQ ID NO.: 43)
AGAACACCGTCGAATGCATGCAAGCCGGTGCGGTGTTCGGCTTCGCCGGG
CTGGTAGACGGGTTGGTAG (Nucleotide 4043309-4043241)
>ST-E00142:243:HVLMVCCXX:8:1215:29812:28664/2
(SEQ ID NO.: 44)
CTACCAACCCGTCTACCAGCCCGGCGAAGCCGAACACCGCACCGGCTTGC
ATGCATTCGACGGTGTTCT (Nucleotide 4043241-4043309)
>ST-E00142:243:HVLMVCCXX:8:1222:8197:67234/1
(SEQ ID NO.: 45)
CGGGGTCATGGTCGCTCTATGCCTCGGCGGCGGCGTTTTCGGGCTGAGCC
TCGGCAAGCACGTCACGCAGAGCGGCTTCTACGACGACGGCAGCCAATCG
GTGCAAGCATCG (Nucleotide 247268-247157)
>ST-E00142:243:HVLMVCCXX:8:1222:8197:67234/2
(SEQ ID NO.: 46)
CGATGCTTGCACCGATTGGCTGCCGTCGTCGTAGAAGCCGCTCTGCGTGA
CGTGCTTGCCGAGGCTCAGCCCGAAAACGCCGCCGCCGAGGCATAGAGCG
ACCATGACCCCG (Nucleotide 247157-247268)
>ST-E00142:243:HVLMVCCXX:8:1223:32826:24216/1
(SEQ ID NO.: 47)
ACGATCTGCCGGGCGGCGGCATGTGTGCGCGGCGCGATGCCATCGACGAG
GCGCGCCAACCGCGGCGCCGGAACCGCCAGGATGACGGCGTCGGCCTGCC
AGCGGCCGCCGGTTTCGTCGCGCAGCACCCAGCCGCGTTCGAGCTGGACC
(Nucleotide 2993118-2993267)
>ST-E00142:243:HVLMVCCXX:8:1223:32857:24233/1
(SEQ ID NO.: 48)
ACGATCTGCCGGGCGGCGGCATGTGTGCGCGGCGCGATGCCATCGACTAG
GCGCGCCAACCGCGGCGCCGGAACCGCCAGGATGACGGCGTCGGCCTGCC
AGCGGCCGCCGGTTTCGTCGCGCAGCACCCAGCCGCGTTCGAGCTGGACC
(Nucleotide 2993118-2993267)
>ST-E00142:243:HVLMVCCXX:8:2105:14519:48757/1
(SEQ ID NO.: 49)
TCACATCCGGGTGGCGGGCAGCCGTCGTCTGCAACGAGGCCGACGGTAGG
CGATGGGTGAGGCCATGGAGGGCAAATGTCCCCATGGTGTATCGATTACG
TACCCTCTTACTTCGTCGGGGTCGCATCGGCATTCGCCTTGCCCGCCTGT
(Nucleotide 1934277-1934128)
>ST-E00142:243:HVLMVCCXX:8:2105:14519:48757/2
(SEQ ID NO.: 50)
CGGGCGACACACCGTCCCGATACATGTCAGCAACCGGGTCGATCGTGGTG
AATGCACAGGCGGGCAAGGCGAATGCCGATGCGACCCCGACGAAGTAAGA
GGGTACGTAATCGATACACCATGGGGACATTTGCCCTCCATGGCCTCACC
(Nucleotide 1934073-1934222)
>ST-E00142:243:HVLMVCCXX:8:2111:10490:53276/1
(SEQ ID NO.: 51)
GCACGCACCCACCTGCACATCGAAATCGTGCCCGGCCTGGCCGCCAGCAG
CGCGGTCCCGACCTATGCCGGGTTGCCGCTGGGTTCGTCGCACACCGTCG
CCGACGTGCGTATC (Nucleotide 603248-603361)
>ST-E00142:243:HVLMVCCXX:8:2111:10490:53276/2
(SEQ ID NO.: 52)
GATACGCACGTCGGCGACGGTGTGCGACGAACCCAGCGGCAACCCGGCAT
AGGTCGGGACCGCGCTGCTGGCGGCCAGGCCGGGCACGATTTCGATGTGC
AGGTGGGTGCGTGC (Nucleotide 603361-603248)
>ST-E00142:243:HVLMVCCXX:8:2111:10500:53258/1
(SEQ ID NO.: 53)
GCACGCACCCACCTGCACATCGAAATCGTGCCCGGCCTGGCCGCCAGCAG
CGCGGTCCCGACCTATGCCGGGTTGCCGCTGGGTTCGTCGCACACCGTCG
CCGACGTGCGTATC (Nucleotide 603248-603361)
>ST-E00142:243:HVLMVCCXX:8:2111:10500:53258/2
(SEQ ID NO.: 54)
GATACGCACGTCGGCGACGGTGTGCGACGAACCCAGCGGCAACCCGGCAT
AGGTCGGGACCGCGCTGCTGGCGGCCAGGCCGGGCACGATTTCGATGTGC
AGGTGGGTGCGTGC (Nucleotide 603361-603248)
>ST-E00142:243:HVLMVCCXX:8:2111:19461:17342/1
(SEQ ID NO.: 55)
CCACGTGGCTACCCATGACCGCGCTCTCAGCGATCTTGTCTTTCTTGACG
TAGTGGAAGTATTTCGGTGTGTCGCTGCCGGTCCCGTCGTCGACGCGTTC
GTCGGCGTCGGTACGTTCAATCGTCTGGGTCTGCATACCTGACATTGTGC
(Nucleotide 3021652-3021801)
>ST-E00142:243:HVLMVCCXX:8:2111:19461:17342/2
(SEQ ID NO.: 56)
TGCCAAGGGCACAATGTCAGGTATGCAGACCCAGACGATTGAACGTACCG
ACGCCGACGAACGCGTCGACGACGGGACCGGCAGCGACACACCGAAATAC
TTCCACTACGTCAAGAAAGACAAGATCGCTGAGAGCGCGGGCATGGGTAG
(Nucleotide 3021809-3021660)
>ST-E00142:243:HVLMVCCXX:8:2116:2676:63085/1
(SEQ ID NO.: 57)
CTTCCGATAAATAGTTAGCCGAATTATATCCTCAGGCATCAATCTCAGCT
CGTCCAATCGAGCA (Nucleotide 1566814-1566751)
>ST-E00142:243:HVLMVCCXX:8:2116:2676:63085/2
(SEQ ID NO.: 58)
TGCTCGATTGGACGAGCTGAGATTGATGCCTGAGGATATAATTCGGCTAA
CTATTTATCGGAAG (Nucleotide 1566751-1566814)
>ST-E00142:243:HVLMVCCXX:8:2117:2960:14564/1
(SEQ ID NO.: 59)
TGATGGCGCTGGTGGAATACTCCGCCGACGAAATCAGAGAAGTGTTCTCC
GACTTCCCCGATCTGGAGGTGTGTGTCTACGCCGCGCCC
(Nucleotide 4258616-4258528)
>ST-E00142:243:HVLMVCCXX:8:2117:2960:14564/2
(SEQ ID NO.: 60)
GGACGCGGCGTAGACACACACCTCCAGATCGGGGAAGTCGGAGAACACTT
CTCTGATTTCGTCGGCGGAGTATTCCACCAGCGCCATCA
(Nucleotide 4258528-4258616)
>ST-E00142:243:HVLMVCCXX:8:2117:6827:70275/1
(SEQ ID NO.: 61)
GGTCGTGCTGGCGGCCACGCCGTGATCGCCAGCTCGAAGACAACGCGCCA
CGCGTGAAGCGGCTCGATCTGGTCGCCGGGCCCAACGGCGCCG
(Nucleotide 444868-444776)
>ST-E00142:243:HVLMVCCXX:8:2117:6827:70275/2
(SEQ ID NO.: 62)
CGGCGCCGTTGGGCCCGGCGACCAGATCGAGCCGCTTCACGCGTGGCGCG
TTGTCTTCGAGCTGGCGATCACGGCGTGGCCGCCAGCACGACC
(Nucleotide 444776-444868)
>ST-E00142:243:HVLMVCCXX:8:2118:14539:66865/1
(SEQ ID NO.: 63)
CAAAGGATTACCAGCGCGAGGACCTGAACCCTGAGTTCTTCGCGGCGTGT
TCTCGGCATCTGCATGGACGTAGCAGACTGTGGTTGTTCCGCTACCAGGG
CACGCCAATTGCCTTCTTTTTGAACGTTTGGGGTGCGGATGAGAACTACA
(Nucleotide 34929-35078)
>ST-E00142:243:HVLMVCCXX:8:2118:14539:66865/2
(SEQ ID NO.: 64)
CAGTATGTAGTTCTCATCCGCACCCCAAACGTTCAAAAAGAAGGCAATTG
GCGTGCCCTGGTAGCGGAACAACCACAGTCTGCTACGTCCATGCAGATGC
CGAGAACACGCCGCGAAGAACTCAGGGTTCAGGTCCTCGCGCTGGTAATC
(Nucleotide 35083-34934)
>ST-E00142:243:HVLMVCCXX:8:2119:2209:19627/1
(SEQ ID NO.: 65)
CGAGGGTGACGACCCGTCGATGACCAACCCCTACATGTACGACGTCGTCG
ACACCAAGCGCGGGGCCCGCAAAAGCTACACCGAAGCCCTGATCGGACGT
GGCGACATCTCGATGAAAGAGGCCGAGGACGCGCTGCGCGACTACCAGGG
(Nucleotide 1390797-1390648)
>ST-E00142:243:HVLMVCCXX:8:2203:18254:2522/1
(SEQ ID NO.: 66)
TTAGCCCACCGTATCGGGCGGCGGGGACTTGCCGGAAACCGACGAACGCC
TCGGCATCCATCCAAATGGCGACGAAAGATCACGGAATTGTCGCGAAACG
AACG (Nucleotide 4082867-4082764)
>ST-E00142:243:HVLMVCCXX:8:2203:24525:40249/1
(SEQ ID NO.: 67)
CTCGAGCAAAAACGTCTTCACCGGTGTTGCCCGCACCCGTAGCCGCCAAC
AACGCGGCATCCAGATCGCGCTGTTGGCTGG (Nucleotide
687626-687545)
>ST-E00142:243:HVLMVCCXX:8:2203:24525:40249/2
(SEQ ID NO.: 68)
CCAGCCAACAGCGCGATCTGGATGCCGCGTTGTTGGCGGCTACGGGTGCG
GGCAACACCGGTGAAGACGTTTTTGCTCGAGG (Nucleotide
687545-687626)
>ST-E00142:243:HVLMVCCXX:8:2209:22871:18573/1
(SEQ ID NO.: 69)
CAAAGGATTACCAGCGCGAGGACCTGAACCCTGAGTTCTTCGCGGCGTGT
TCTCGGCATCTGCATGGACGTAGCAGACTGTGGTTGTTCCGCTACCAGGG
CACGCCAATTGCCGTCTTTTTGAACGGTTGGGGTGCGGATGAGACCTACA
(Nucleotide 34929-35078)
>ST-E00142:243:HVLMVCCXX:8:2209:22871:18573/2
(SEQ ID NO.: 70)
CAGTATGTAGTTCTCATCCGCACCCCAAACGTTCAAAAAGAAGGCAATTG
GCGTGCCCTGGTAGCGGAACAACCACAGTCTGCTACGTCCATGCAGATGC
CGAGAACACGCCGCGAAGAACTCAGGGGTCAGGTCCTCGCGCTGGTAATC
(Nucleotide 35083-34934)
>ST-E00142:243:HVLMVCCXX:8:2210:28919:55034/2
(SEQ ID NO.: 71)
GGCTAGGCGCGAAAAGCGCGTGTTCGACATGTTCGGAGATCGCATGAATA
AAAACGATGTGAGATTCCTGGATTCGCCCGGTGTCGCGTGACGGGACGTT
GATCAAGAAATCTGCGAATTCTGCCAGCTGGCCGCCGGAGTCGCCCGTCA
(Nucleotide 137904-137755)
>ST-E00142:243:HVLMVCCXX:8:2221:11617:15637/1
(SEQ ID NO.: 72)
CCACTTCGGGTTCACCCTCGGCGGCAACGAGCGTGTCGAGCATCCGTTCA
CACTGCGCCGCGGTGATGGCGTAGCCGTCGGGT (Nucleotide
4178708-4178626)
>ST-E00142:243:HVLMVCCXX:8:2221:11617:15637/2
(SEQ ID NO.: 73)
ACCCGACGGCTACGCCATCACCGCGGCGCAGTGTGAACGGATGCTCGACA
CGCTCGTTGCCGCCGAGGGTGAACCCGAAGTGG (Nucleotide
4178626-4178708)

The above identified target sequences in the genomic nucleic acid sequence of Mycobacterium tuberculosis are derived from whole genome sequencing from the plasma sample of a patient who has pulmonary tuberculosis. It is worth noting that it is generally unexpected to detect tuberculosis DNA signal from circulating plasma through whole genome sequencing. It is also to be noted that all of these target sequences fall within the above 17 CpG islands identified for Mycobacterium tuberculosis.

Examples of primers that can be used for the amplification of a target sequence in the CpG island of the genomic nucleic acid sequence of Mycobacterium tuberculosis include but are not limited to: forward primer GGCTGTGGGTAGCAGACC (SEQ ID NO.: 74) and ACCTGAAAGACGTTATCCACCAT (SEQ ID NO.: 75), reverse primer CGGGTCCAGATGGCTTGC (SEQ ID NO.: 76) and CGGCTAGTGCATTGTCATAGGA (SEQ ID NO.: 77).

The amplification of a target sequence in the CpG island of the genomic nucleic acid sequence of Mycobacterium tuberculosis may also comprise the use of a probe. In some examples, the probe used comprises the sequence of TGTCGACCTGGGCAGGGTTCG (SEQ ID NO.: 78) and TCCGACCGCGCTCCGACCGACG (SEQ ID NO.: 79).

In one example, the probe used comprises a component comprises at least one detectable label. In one example, the detectable label is capable of producing an optical signal. In one example, the detectable label comprises a fluorophore. Examples of fluorophores include but are not limited to fluorescent proteins, for example GFP (green fluorescent protein), YFP (yellow fluorescent protein), RFP (red fluorescent protein); non-protein fluorophores selected from the group consisting of xanthene derivatives (for example, fluorescein, rhodamine, Oregon green, eosin, 6-carboxyfluorescein and Texas red); cyanine derivatives (for example, cyanine, indocarbocyanine, oxacarbocyanine, thiacarbocyanine, and merocyanine), squaraine derivatives and ring-substituted squaraines, including Seta, SeTau, and Square dyes, naphthalene derivatives (dansyl and prodan derivatives), coumarin derivatives, oxadiazole derivatives (for example pyridyloxazole, nitrobenzoxadiazole and benzoxadiazole), anthracene derivatives (for example anthraquinones, DRAQ5, DRAQ7 and CyTRAK Orange), pyrene derivatives(for example cascade blue), oxazine derivatives (for example, Nile red, Nile blue, cresyl violet, oxazine 170), acridine derivatives (for example proflavin, acridine orange, acridine yellow), arylmethine derivatives (for example auramine, crystal violet, malachite green), tetrapyrrole derivatives (for example porphin, phthalocyanine, bilirubin) and derivatives thereof. In some specific examples, the fluorophore is selected from the group consisting of FAM (carboxyfluorescein), TET (carboxy-2′,4,7,7′-tetrachlorofluorescein succinimidyl ester), HEX (carboxy-2,4,4,5,7,7-hexachlorofluorescein succinimidyl ester), ROX (carboxy-X-rhodamine) and NED. In one specific example, the fluorophore is FAM (carboxyfluorescein), or 6-FAM (6-carboxyfluorescein).

In one example, the at least one detectable label is capable of producing a changeable signal. The changeable signal may be produced upon the hybridization of the probe to the target sequence. For example, the signal may be detectable before the probe binds to the target sequence, and upon the hybridization of the probe to the target sequence, the signal is reduced in strength or becomes completely undetectable. In another example, the detectable signal may be produced only upon the hybridization of the probe to the target sequence, or the strength of the detectable signal may be increased upon the hybridization of the probe to the target sequence.

In one example, the component comprises two detectable labels. In one example, the two detectable labels function independently, while in another example, the two detectable labels are an interactive pair of labels. The interactive pair of labels are capable of generating a changeable signal. For example, the signal may be detectable before the probe binds to the target sequence, and upon the hybridization of the probe to the target sequence, the signal is reduced in strength or becomes completely undetectable. In another example, the detectable signal may be produced only upon the hybridization of the probe to the target sequence, or the strength of the detectable signal may be increased upon the hybridization of the probe to the target sequence. In one specific example, the detectable signal is not generated when both detectable labels are linked together by the probe sequence. Once at least one detectable label is cleaved from the probe, the detectable signal is generated.

In some examples, the interactive pair of labels may comprise a fluorophore and a quencher pair. In one specific example, the fluorophore is located at the 5′ end of the probe, and the quencher is located at the 3′end of the probe. Examples of quenchers include but are not limited to TAMRA (tetramethylrhodamine), TaqMan® MGB (minor groove binder) and BHQ™ (Black Hole Quencher™). In one specific example, the fluorophore is FAM (carboxyfluorescein), more particularly 6-FAM (6-carboxyfluorescein), and the quencher is TAMRA (tetramethylrhodamine).

The amplification of the target sequence in the above methods may be carried out via a polymerase chain reaction (PCR). Examples of PCRs include but are not limited to real-time polymerase chain reaction, digital polymerase chain reaction, quantitative polymerase chain reaction, qualitative polymerase chain reaction, quantitative real-time polymerase chain reaction, or quantitative reverse transcription polymerase chain reaction.

Real-time PCR monitors the amplification of a targeted DNA molecule during the PCR, i.e. in real-time, and not at its end, as in conventional PCR. Real-time PCR can be used quantitatively (Quantitative real-time PCR), and semi-quantitatively, i.e. above/below a certain amount of DNA molecules (Semi quantitative real-time PCR). Quantitative Real-Time PCR (qrt-PCR) methods use fluorescent dyes or fluorophore-containing DNA probes to measure the amount of amplified product as the amplification progresses.

Digital PCR (dPCR) simultaneously amplifies thousands of samples, each in a separate droplet within an emulsion.

Quantitative PCR (qPCR) is used to measure the specific amount of target DNA (or RNA) in a sample. By measuring amplification only within the phase of true exponential increase, the amount of measured product more accurately reflects the initial amount of target. Special thermal cyclers are used that monitor the amount of product during the amplification.

Qualitative PCR refers to a PCR method used to detect the present or absence of target DNA (RNA) in a sample without quantifying the amount present.

Reverse Transcription PCR is used to reverse-transcribe and amplify RNA to cDNA. PCR is preceded by a reaction using reverse transcriptase, an enzyme that converts RNA into cDNA. The two reactions may be combined in a tube, with the initial heating step of PCR being used to inactivate the transcriptase. RT-PCR is widely used in expression profiling, which detects the expression of a gene. It can also be used to obtain sequence of an RNA transcript, which may aid the determination of the transcription start and termination sites and facilitate mapping of the location of exons and introns in a gene sequence.

The amplified product obtained using the methods described above can be purified and the resulting purified product can be quantified using conventional nucleic acid purification methods and quantification methods. Examples of nucleic acid purification methods include but are not limited to gel electrophoresis followed by gel extraction, and silica based membrane technologies. Examples of methods to quantify the purified nucleic acid include but are not limited to spectrophotometric analysis and analysis using fluorescent dye tagging. Various kits and systems are commercially available for the purification and quantification of amplified. nucleic acid products.

The copy number of BamHI-W in the amplified product can be calculated using the following formula:

$$Copy Number of BamHI - W = \frac{DNA Quantity ( ng ) \times Avogradro \text{’} s Number}{88390.29 ( Da ) \times {10}^{9}}$$

The methods described herein have better sensitivity and specificity compared to the currently known methods of detecting microorganisms. Specifically, for the detection of EBV, in terms of sensitivity, the lowest concentration of EBVs in a sample that can be detected using the methods described herein is 100 International Unity (IU)/ml or sample, or 90 IU/ml of sample, or 80 IU/ml of sample, or 70 IU/ml of sample, or 60 IU/ml of sample, or 50 IU/ml of sample, or 40 IU/ml of sample, or 30 IU/ml of sample, or 20 IU/ml of sample, or 10 IU/ml of sample, or 5 IU/ml of sample, or 1 IU/ml of sample.

The term “International Unit” or “IU” as used herein refers to the first WHO International Standard for Epstein-Barr Virus for Nucleic Acid Amplification Techniques defined by the National Institute for Biological Standards and Control (NIBSC) (NIBSC Code No. 09/260).

As described above, the genome of an EBV typically contains six to twenty copies of the BamHI-W sequence. Therefore higher sensitivity can be achieved when the detection of EBV is based on the detection of the BamHI-W region. However, the variability of BamHI-W copy numbers in different EBV strains has been considered as a challenged in assay comparison and standardization between laboratories. Therefore, a new method of standardizing the number of copies of BamHI-W to the amount of EBV in a sample has been developed in the present disclosure to solve this problem. The sequence of BamHI-W standard was incorporated as an insert in plasmid which was propagated in competent bacteria cells, such as E. coli. Single colony was picked and grown for scale-up production of the plasmids. Bacteria were harvested and plasmids were extracted and quantified.

The BamHI-W standard plasmid obtained carries only BamHI-W sequence of EBV whereas NIBSC standard is the whole genome sequence of EBV. The presence of other genes in EBV genome might interfere with the amplification of BamHI-W or generate higher background signals as compared to the BamHI-W standard plasmid. In addition, the BamHI-W standard plasmid obtained carries one BamHI-W copy, allowing absolute quantification of BamHI-W. This is not feasible in the case of NIBSC standards because number of BamHI-W copies is unknown in EBV genome.

Using the constructed standard plasmid of BamHI-W, it has been derived that 1 IU (International Unit) of EBV as defined by the NIBSC standard equals to about 1.38 copies of BamHI-W. This conversion allows standardization between BamHI-W assay of the present disclosure and other assays, allowing comparison of test results across various laboratories that use different types of assay for EBV quantification.

The method of detecting and/or quantifying a target nucleic acid sequence of the EBV can be used alone or in combination with other available methods of detecting and/or quantifying EBV.

In another aspect, there is provided a method of detecting a disease associated with microorganism infection, or risk of developing a disease associated with microorganism infection in a subject, comprising detecting and/or quantifying the presence of a nucleic acid sequence of the microorganism using the method of the present invention in a sample obtained from the subject, wherein the presence of the nucleic acid sequence of the microorganism in the sample indicates that the subject has a disease associated with microorganism infection or is at risk of developing a disease associated with microorganism infection.

In a further aspect, there is provided a method of detecting and treating a disease associated with microorganism infection, comprising: (i) detecting and/or quantifying the presence of a nucleic acid sequence of the microorganism using the method of the present invention in a sample obtained from the subject, wherein the presence of the nucleic acid sequence of the microorganism in the sample indicates that the subject has a disease associated with microorganism; (ii) administering to the subject a medicament suitable for the treatment of the disease associated with the microorganism.

In yet a further aspect, there is provided a method of predicting the treatment outcome of a disease associated with microorganism infection in a patient, comprising: (i) quantifying the nucleic acid sequence of the microorganism in a sample collected from the patient before treatment or before a treatment step, and quantifying the nucleic acid sequence of the microorganism in a sample collected from the same patient after treatment or after a treatment step; (ii) comparing the amount of the nucleic acid sequence of the microorganism in the sample before and after treatment or a treatment step, wherein a decrease in the amount of the nucleic acid sequence of the microorganism in the sample after treatment or a treatment step indicates that treatment outcome of the disease associated with microorganism infection in the patient is positive, wherein the quantifying of the nucleic acid sequence of the microorganism in the sample is performed according to the method of the present invention.

The term “disease associated with microorganism infection” as used herein refers to any disease that can be caused by a microorganism, in particular a pathogenic microorganism as described herein.

In one specific example, the disease is EBV-associated disease, in particular EBV-associated cancers. Examples of EBV-associated cancers include but are not limited to nasopharyngeal carcinoma (NPC), gastric cancer, Hodgkin's lymphoma and Burkitt's lymphoma. In one specific example, the EBV-associated cancer is NPC.

The term “sample” used herein refers to a biological sample, or a sample that comprises at least some biological materials such as nucleic acid molecules, more particularly cell free DNAs (cfDNAs). The biological samples may include liquid samples, such as whole blood, blood serum, blood plasma, buffy coat, peripheral blood mononuclear cells (PBMCs), cerebrospinal fluid, central spinal fluid, lymph fluid, cystic fluid, sputum, stool, pleural effusion, mucus, pleural fluid, ascitic fluid, amniotic fluid, peritoneal fluid, saliva, bronchial washes and urine. In specific examples, the biological sample is a blood sample or blood plasma sample. In some other specific examples, the biological sample is not a urine sample. Nucleic acids can be extracted from a biological sample using any method known to those of skill in the art.

In one specific example, the subject from which the sample is obtained is of Asian ethnicity.

The term cell free DNA (cfDNA) is used herein to refer to DNA that is found in the circulating system of a subject. cfDNAs can be microorganism cfDNAs, or cfDNAs directly released from mammalian cells, in particular abnormal cells such as cancer cells. Microorganism cfDNAs may be released from the circulating cell free microorganisms, or from microorganisms present in mammalian cells. cfDNAs directly released from mammalian cells could have been incorporated into the DNAs of the mammalian cells as a result of mammalian cells infection caused by the microorganisms.

cfDNAs which are residing within the CpG island is more stable and less susceptible to degradation and hence more likely to be detected. Therefore when a sample contains both microorganism cfDNAs and human cfDNAs, targeting microorganism cfDNAs residing within the CpG island makes the microorganism cfDNAs more likely to be detected amongst the presence of human cfDNAs.

In some examples, it is preferred to select target sequences within a nucleic acid sequence that occurs in multiple repeats in a microorganism, in order to make detection and/or quantification more feasible, especially in sample with low content of the microorganism.

The presence of the DNA sequence of a microorganism in a sample from a subject can also be used as an indication of the stage of the disease associated with the microorganism. In one specific example, the presence of an EBV DNA sequence in a sample from a subject can be used as an indication of the stage of the EBV- associated cancers.

The term “treatment” as used herein refers to any methods or substances or combination thereof, which remedy a disease state or symptoms, prevent the establishment of disease, or otherwise prevent, hinder, retard, or reverse the progression of disease or other undesirable symptoms. In particular, the disease is an EBV-associated disease, such as EBV-associated cancers, which include but are not limited to nasopharyngeal carcinoma, gastric cancer, Hodgkin's lymphoma and Burkitt's lymphoma. Types of cancer treatment generally include chemotherapy, radiation therapy, immunotherapy, and targeted therapy.

The term “decrease” or “reduce” and their grammatically variance refers to a decrease in the level of EBV in a sample collected from the patient after treatment or a treatment step as compared to the level of EBV in a sample collected from the same patient before treatment or before a treatment step. In some examples, the level of EBVs in the sample collected after treatment or a treatment step is reduced by at least 10%, at least 20%, at least 30%, at least 40%, at least 50%, at least 60%, at least 70%, at least 80%, at least 90% or at least 95% as compared to the level of EBVs in a sample collected from the same patient before treatment or before a treatment step.

The present disclosure also provides a kit for detecting and/or quantifying the presence of a target nucleic acid of a microorganism in a sample, where the kit can be used according to the methods of the present invention.

In one aspect, there is provided a kit for detecting and/or quantifying the nucleic acid sequence of a microorganism in a sample obtained from a subject, comprising a pair of oligonucleotide primers specific for the amplification of a target sequence in a CpG island of the nucleic acid of the microorganism.

In one example, the kit further comprises a probe capable of binding to the target sequence. In some examples, the probe is any probe as described above.

In yet a further example, there is provided one or more oligonucleotide primers for the amplification of a target sequence in the CpG island of the nucleic acid of a microorganism.

The sequences as described herein are from the 5′ to the 3′ direction, unless specified otherwise.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### Experimental Section

### EXAMPLE 1

### Comparison of Sensitivity and Specificity between EBV cfDNA Assays

Benchmarking of the EBV cfDNA was conducted using comparison against results from a College of American Pathologists (CAP)-accredited laboratory as well as WHO-approved international EBV standards.

The clinical sensitivity and specificity of the three EBV cfDNA assays was benchmarked against an in-house EBV cfDNA assay targeting EBNA1 in a College of American Pathologists (CAP)-accredited clinical-grade laboratory at the Singapore General Hospital (SGH), with known analytical performance reported as a sensitivity of 79% and specificity of 100%. Out of 46 NPC patients (Table 1), 31 (69%) were reported to be EBV-positive, and 14 (31%) were reported to be EBV-negative (1 case was not reported due to logistic reasons). Of 31 EBV-positive patients on the clinical-grade assay, both BamHI-W qPCR and EBNA1-dPCR assays showed 100% matching positivity, whereas the EBNA1-qPCR assay showed 80% match. Of the 14 EBV-negative patients, the BamHI-W qPCR, EBNA1-dPCR and EBNA1-qPCR assay reported 9, 7, and 5 positive cases. Overall, all three EBV cfDNA assays demonstrate high clinical sensitivity and specificity, with particularly high sensitivity shown at baseline for the BamHI-W qPCR assay.

TABLE 2
Patient characteristics

| Characteristic | No. of patients (%) | |
|---|---|---|
| Total | 46 | (100.0) |
| Gender | | |
| Male | 38 | (82.6) |
| Female | 8 | (17.4) |
| Age (median, 50; | | |
| range, 23-80) | | |
| ≤50 | 24 | (52.2) |
| >50 | 22 | (47.8) |
| T-classification | | |
| 1 | 16 | (34.8) |
| 2 | 5 | (10.9) |
| 3 | 19 | (41.3) |
| 4 | 6 | (13.0) |
| N-classification | | |
| 0 | 8 | (17.4) |
| 1 | 17 | (37.0) |
| 2 | 15 | (32.6) |
| 3 | 6 | (13.0) |
| M-classification | | |
| 0 | 43 | (93.5) |
| 1 | 3 | (6.5) |
| AJCC 7^{th} Stage | | |
| I | 8 | (17.4) |
| II | 8 | (17.4) |
| III | 18 | (39.1) |
| IV | 12 | (26.1) |
| Treatment | | |
| Radiotherapy alone | 16 | (34.8) |
| Chemo-Radiotherapy | 27 | (58.7) |
| Unknown | 3 | (6.5) |
| Adjuvant | | |
| Chemotherapy | | |
| Yes | 3 | (6.5) |
| No | 43 | (93.5) |

Abbreviations:
AJCC, American Joint Committee on Cancer

The only available WHO-approved international EBV standard was used to benchmark the sensitivity and specificity of the three EBV cfDNA assays. The BamHI-W qPCR assay demonstrated the highest reproducible sensitivity. The lowest EBV concentration detected in triplicates was 100 IU/mL for BamHI-W qPCR assay and 1,000 IU/mL for both EBNA1 assays (Table 2). The BamHI-W qPCR assay was also able to detect positive signal in one replicate of the standard containing 1 IU/mL, whereas EBNA1 assays were not able to. In addition, all assays produced no false-positive detection in five EBV-free standards, indicating their high specificity against EBV cfDNA.

TABLE 3
Sensitivity and Specificity of EBV cfDNA Quantitative Assays

| Spike-in | Total | Number of Positive Spike-in Standards | | |
|---|---|---|---|---|
| Standards | Number of | BamHI-W qPCR | EBNA1-qPCR | EBNA1-dPCR |
| (IU/mL) | Samples | Assay | Assay | Assay |
| 1,000,000 | 3 | 3 | 3 | 3 |
| 1,000 | 3 | 3 | 3 | 3 |
| 100 | 3 | 3 | 1 | 2 |
| 10 | 3 | 2 | 1 | 0 |
| 1 | 3 | 1 | 0 | 0 |
| 0 | 3 | 0 | 0 | 0 |
| Blank | 2 | 0 | 0 | 0 |

Abbreviations:
EBV, Epstein-Barr virus

The IU of NIBSC standards is derived from a mean value of highly variable EBV copy number measured by various qPCR assays of 28 laboratories in the world. These assays employ different DNA extraction methods, and target a wide range of genes, including a single-copy gene, EBNA1, and a multiple-repeat gene, BamHI-W. However, since dPCR was not included in the evaluation, the relationship between EBV copy number as obtained by dPCR and IU is less clear. Moreover, since the number of BamHI-W fragments varies in different EBV isolates, a fixed conversion ratio of BamHI-W copies to IU will not be always accurate in different patients' sample. Therefore, the NIBSC standards were only used in this study for comparison of sensitivity and specificity between EBV cfDNA assays. The subsequent data were to be reported in copy number of respective EBV targets.

### EXAMPLE 2

### Relationship between NPC Circulating Biomarkers in Pre-Treatment Samples

Among EBV cfDNA quantitation approaches, BamHI-W qPCR assay yielded the highest concentration of EBV cfDNA levels: 2.4 to 37.7-fold higher than EBNA1-qPCR assay and 2.2 to 25.5-fold higher than EBNA1-dPCR assay (Table 3).

All samples detected EBV-positive by both EBNA1 assays were also detected positive for EBV by BamHI-W assay. The detection rates of canonical CTCs and potential CTCs are 76% and 94% in pre-treatment samples respectively. Overall, potential CTC count was higher and weakly correlated to canonical CTC count (r²=0.21, P-value=<0.01). No correlation was observed between each type of CTC count and EBV cfDNA levels quantified by different assays. In contrast, among the EBV cfDNA assays, strong correlation was observed between BamHI-W qPCR and EBNA1-dPCR assays (r²=0.99, P-value<0.0001), but not between BamHI-W and EBNA1-qPCR assays (r²=0.03, P-value=0.29) nor between EBNA1-qPCR and -dPCR assays (r²=0.06, P-value=0.11). This result corresponded with the similar detection rate of BamHI-W qPCR (89%) and EBNA1-dPCR (85%) assays, with the detection rate of EBNA1-qPCR assay being 67%.

TABLE 4
Quantitative levels of NPC circulating biomarkers in 46 pre-treatment samples

| | | | Pre-Treatment | | | | |
|---|---|---|---|---|---|---|---|
| | | Status | | | EBNA1- | | |
| | AJCC | on | BamHI-W | EBNA1- | dPCR | Canonical | Potential |
| Patient | 7^{th} | Follow- | qPCR Assay | qPCR Assay | Assay | CTCs | CTCs |
| ID | Stage | up | (copies/mL) | (copies/mL) | (copies/mL) | (cells/mL) | (cells/mL) |
| 001 | III | NED | 70,569 | 5,416 | 9,484 | 0 | 3 |
| 002 | III | NED | 9,728 | 385 | 1,109 | 0 | 12 |
| 003 | III | NED | 10,631 | 855 | 1,376 | NA | NA |
| 004 | III | NED | 507 | 30 | 43 | 0 | 0 |
| 005 | IV | NED | 1,324 | 80 | 168 | 5 | 13 |
| 006 | I | NED | 0 | 0 | 0 | 4 | 13 |
| 007 | I | NED | 21 | 0 | 9 | 0 | 20 |
| 008 | I | NED | 107 | 0 | 0 | 0 | 14 |
| 009 | III | DOD | 18,572 | 5,425 | 3,636 | NA | NA |
| 010 | IV | AWD | 1,249 | 36 | 132 | 3 | 4 |
| 011 | II | NED | 23,507 | 2,838 | 3,656 | 2 | 1 |
| 012 | IV | DOD | 99,379 | 18,816 | 14,199 | 6 | 146 |
| 013 | II | NED | 301 | 0 | 28 | 0 | 0 |
| 014 | I | NED | 0 | 0 | 0 | 1 | 12 |
| 017 | II | NED | 67 | 0 | 21 | 50 | 134 |
| 018 | IV | NED | 441,316 | 13,565 | 50,081 | 11 | 29 |
| 019 | I | NED | 162 | 0 | 13 | 18 | 44 |
| 020 | III | NED | 4,860 | 0^{a} | 804 | 16 | 17 |
| 021 | III | NED | 1,236 | 33 | 49 | 37 | 76 |
| 022 | III | NED | 44,918 | 1,964 | 3,949 | NA | NA |
| 023 | III | NED | 29,006 | 777 | 3,272 | NA | NA |
| 024 | I | NED | 0 | 49 | 0 | 31 | 63 |
| 025 | IV | NA | 290,961 | 16,727 | 53,740 | 5 | 68 |
| 026 | IV | NED | 1,157 | 121 | 230 | 16 | 83 |
| 027 | III | NED | 6,687 | 431 | 1,356 | 4 | 21 |
| 028 | I | NED | 360 | 0 | 53 | 1 | 7 |
| 029 | III | NED | 6,072 | 303 | 816 | 17 | 47 |
| 030 | III | NED | 8,226 | 714 | 1,095 | 0 | 15 |
| 031 | IV | NED | 9,507 | 442 | 670 | 4 | 19 |
| 032 | II | NED | 92 | 0 | 42 | 3 | 67 |
| 033 | IV | DOD | 1,743,700 | 0^{a} | 193,125 | 6 | 44 |
| 034 | III | NED | 9,043 | 447 | 1,279 | 1 | 63 |
| 035 | II | NED | 146 | 21 | 0 | 1 | 14 |
| 036 | III | NED | 105 | 0 | 0 | 0 | 182 |
| 037 | II | NED | 669 | 0 | 63 | 3 | 13 |
| 038 | IV | AWD | 81 | 26 | 105 | 3 | 31 |
| 039 | III | DOD | 6,613 | 439 | 780 | 1 | 15 |
| 040 | III | NED | 5,106 | 623 | 1,125 | 1 | 14 |
| 041 | II | NED | 331 | 84 | 46 | NA | NA |
| 042 | III | NED | 2,156 | 171 | 241 | NA | NA |
| 043 | III | NED | 56,490 | 8,829 | 9,894 | NA | NA |
| 044 | IV | DOD | 88,432 | 12,074 | 16,850 | NA | NA |
| 045 | IV | NED | 33,057 | 6,014 | 5,319 | NA | NA |
| 046 | I | NED | 131 | 55 | 7 | NA | NA |
| 047 | IV | NED | 0 | 0 | 0 | NA | NA |
| 048 | II | NED | 0 | 0 | 7 | NA | NA |

Abbreviations:
NPC, nasopharyngeal carcinoma;
AJCC, American Joint Committee on Cancer;
CTCs, circulating tumour cells;;
NED, no evidence of disease;
AWD, alive with disease;
DOD, dead of disease;
NA, data are not available
^{a}PCR inhibition

### EXAMPLE 3

### Relationship between NPC Circulating Biomarkers and Clinical Stage

The clinical stages were re-classified to three groups; stage I, stage II-III, and stage IV (Table 4). The combination of stage-II and -III NPC patients was in the light of long-term 5-year follow-up data from Singapore showing similar survival outcomes using modern treatment approaches¹³. The EBV cfDNA levels in three assays strongly correlated with clinical stages. In contrast, there was no statistically significant relationship between CTCs and clinical stages. These results indicated a strong association between NPC clinical stage and EBV cfDNA, but not CTCs.

TABLE 5
Relationship between NPC circulating biomarkers and clinical
stages in pre-treatment samples

| NPC | Mean Values | | | LR Chi- | Degree | |
|---|---|---|---|---|---|---|
| circulating | | Stage | Stage | Square | of | |
| biomarkers | Stage I | II-III | IV | Values^{a} | Freedom | P-Values^{a} |
| BamHI-W qPCR | 98 | 12,140 | 225,847 | 14.15 | 1 | 0.0002^{b} |
| Assay | | | | | | |
| (copies/mL) | | | | | | |
| EBNA1-qPCR | 13 | 1,146 | 5,658 | 10.84 | 1 | 0.0010^{b} |
| Assay | | | | | | |
| (copies/mL) | | | | | | |
| EBNA1-dPCR | 10 | 1,699 | 27,885 | 14.52 | 1 | 0.0001^{b} |
| Assay | | | | | | |
| (copies/mL) | | | | | | |
| Canonical CTC | 8 | 8 | 7 | 0.05 | 1 | 0.8250 |
| Enumeration | | | | | | |
| (cells/mL) | | | | | | |
| Potential CTC | 25 | 39 | 49 | 1.07 | 1 | 0.3000 |
| Enumeration | | | | | | |
| (cells/mL) | | | | | | |

Abbreviations:
NPC, nasopharyngeal carcinoma;
CTCs, circulating tumour cells
^{a}Likelihood ratio Chi-square and P-values were determined using logistic ordinal regression for the prediction of NPC clinical stage, given the levels of NPC circulation biomarkers
^{b}P-values <0.05 were considered statistically significant.

### EXAMPLE 4

### Relationship between NPC Circulating Biomarkers and Treatment Outcome

Decreased EBV cfDNA levels were observed in all EBV-positive patients following treatment, strongly correlating with the local radiological response (Table 5). To evaluate the predictive value of NPC circulating biomarkers for short-term radiological response, we determined that EBV cfDNA levels were significantly reduced after treatment (Wilcoxon's signed rank testing p-value<0.001 for all three techniques BamHI-W qPCR, EBNA1-dPCR and EBNA1-qPCR assay). In contrast, for both canonical and potential CTCs, decrease was not significant (p=0.07 and 0.54 respectively). The stratified analysis performed on patients undergoing radiotherapy and chemo-radiotherapy showed the magnitude of decrease of canonical CTCs pre- and post-treatment in each group remains insignificant (Table 6). Overall, our results show that EBV cfDNA level correlation with short-term radiological response was much stronger than that of potential or canonical CTC counts.

TABLE 6
Quantitative levels of NPC circulating biomarkers in 28 matched samples

| | | | | BamHI-W | | EBNA1- | | EBNA1- | | | | | |
|---|---|---|---|---|---|---|---|---|---|---|---|---|---|
| | | | | qPCR | | qPCR | | dPCR | | Canonical | | Potential | |
| | | | | Assay | | Assay | | Assay | | CTCs | | CTCs | |
| | | Post- | | (copies/mL) | | (copies/mL) | | (copies/mL) | | (cells/mL) | | (cells/mL) | |
| | | Treatment | | Pre- | Post- | Pre- | Post- | Pre- | Post- | Pre- | Post- | Pre- | Post- |
| | AJCC | Radiological | Status on | Treat- | Treat- | Treat- | Treat- | Treat- | Treat- | Treat- | Treat- | Treat- | Treat- |
| Patient ID | 7^{th} Stage | Response | Follow-up | ment | ment | ment | ment | ment | ment | ment | ment | ment | ment |
| 006 | I | CR | NED | 0 | 0 | 0 | 0 | 0 | 0 | 4 | 3 | 13 | 88 |
| 014 | I | CR | NED | 0 | 0 | 0 | 0 | 0 | 0 | 1 | 5 | 12 | 5 |
| 024 | I | PR | NED | 0 | 0 | 49 | 0 | 0 | 0 | 31 | 1 | 63 | 3 |
| 007 | I | CR | NED | 21 | 0 | 0 | 0 | 9 | 0 | 0 | 0 | 20 | 103 |
| 008 | I | CR | NED | 107 | 0 | 0 | 0 | 0 | 0 | 0 | 3 | 14 | 14 |
| 019 | I | nCR | NED | 162 | 42 | 0 | 0 | 13 | 0 | 18 | 22 | 44 | 148 |
| 028 | I | CR | NED | 360 | 0 | 0 | 0 | 53 | 14 | 1 | 0 | 7 | 13 |
| 017 | II | PR | NED | 67 | 0 | 0 | 0 | 21 | 7 | 50 | 16 | 134 | 220 |
| 032 | II | PR | NED | 92 | 0 | 0 | 0 | 42 | 7 | 3 | 0 | 67 | 15 |
| 035 | II | nCR | NED | 146 | 0 | 21 | 0 | 0 | 0 | 1 | 0 | 14 | 23 |
| 013^{b} | II | CR | NED | 301 | 0 | 0 | 0 | 28 | 0 | 0 | 3 | 0 | 26 |
| 011^{b} | II | CR | NED | 23,507 | 27 | 2,838 | 0 | 3,656 | 0 | 2 | 6 | 1 | 9 |
| 004^{b} | III | CR | NED | 507 | 0 | 30 | 0 | 43 | 0 | 0 | 4 | 0 | 26 |
| 021^{b} | III | nCR | NED | 1,236 | 0 | 33 | 0 | 49 | 0 | 37 | 2 | 76 | 33 |
| 029^{b} | III | nCR | NED | 6,072 | 0 | 303 | 0 | 816 | 0 | 17 | 0 | 47 | 6 |
| 027^{b} | III | CR | NED | 6,687 | 0 | 431 | 0 | 1,356 | 0 | 4 | 1 | 21 | 18 |
| 030^{b} | III | nCR | NED | 8,226 | 0 | 714 | 0 | 1,095 | 0 | 0 | 0 | 15 | 0 |
| 002^{b} | III | nCR | NED | 9,728 | 0 | 385 | 0 | 1,109 | 7 | 0 | 0 | 12 | 150 |
| 003^{b} | III | CR | NED | 10,631 | 0 | 855 | 0 | 1,376 | 0 | NA | NA | NA | NA |
| 009 | III | PD | DOD | 18,572 | 131 | 5,425 | 0 | 3,636 | 35 | NA | NA | NA | NA |
| 001^{b} | III | CR | NED | 70,569 | 0 | 5,416 | 0 | 9,484 | 0 | NA | NA | NA | NA |
| 023^{b} | III | CR | NED | 29,006 | 47 | 777 | 0 | 3,272 | 6 | NA | NA | NA | NA |
| 022^{b} | III | nCR | NED | 44,918 | 0 | 1,964 | 0 | 3,949 | 13 | NA | NA | NA | NA |
| 026^{b} | IV | PR | NED | 1,157 | 0 | 121 | 0 | 230 | 7 | 16 | 2 | 83 | 6 |
| 010^{b} | IV | PR | AWD | 1,249 | 0 | 36 | 0 | 132 | 0 | 3 | 0 | 4 | 3 |
| 005^{b} | IV | nCR | NED | 1,324 | 0 | 80 | 0 | 168 | 0 | 5 | 1 | 13 | 27 |
| 012^{b} | IV | PR | DOD | 99,379 | 24,577 | 18,816 | 2,529 | 14,199 | 5,107 | 6 | 1 | 146 | 35 |
| 018^{b} | IV | PR | NED | 441,316 | 0 | 13,565 | 0 | 50,081 | 0 | 11 | 6 | 29 | 61 |

| | | | | | | | | | | |
|---|---|---|---|---|---|---|---|---|---|---|
| Mean | 27,690 | 887 | 1,852 | 90 | 3,386 | 186 | 9 | 3 | 36 | 45 |

| | | | | | |
|---|---|---|---|---|---|
| P-Values^{a} | <0.001 | <0.001 | <0.001 | 0.07 | 0.54 |

Abbreviations:
NPC, nasopharyngeal carcinoma;
AJCC, American Joint Committee on Cancer;
CTCs, circulating tumour cells;
nCR, near complete response;
CR, complete response;
PR, partial response;
PD, progressive disease;
NED no evidence of disease;
AWD, alive with disease;
DOD, dead of disease;
NA, data are not available
^{a}P-Values were calculated using the Wilcoxon's signed rank testing and values <0.05 were considered statistically significant.

TABLE 7
Stratified analysis of CTC enumeration in NPC pre- and post-treatment samples

| | Radiotherapy | | | | Chemo-Radiotherapy | | | | All Treatment | | | |
|---|---|---|---|---|---|---|---|---|---|---|---|---|
| | (n = 10) | | | | (n = 13) | | | | (n = 23) | | | |
| | Canonical | | Potential | | Canonical | | Potential | | Canonical | | Potential | |
| | CTCs | | CTCs | | CTCs | | CTCs | | CTCs | | CTCs | |
| | Pre | Post | Pre | Post | Pre | Post | Pre | Post | Pre | Post | Pre | Post |
| Mean | 11 | 5 | 39 | 63 | 8 | 2 | 34 | 31 | 9 | 3 | 36 | 45 |

| | | | | | | |
|---|---|---|---|---|---|---|
| P-value | 0.59 | 0.19 | 0.07 | 0.78 | 0.07 | 0.54 |

### EXAMPLE 5

### Relationship between NPC Circulating Biomarkers and Overall Survival

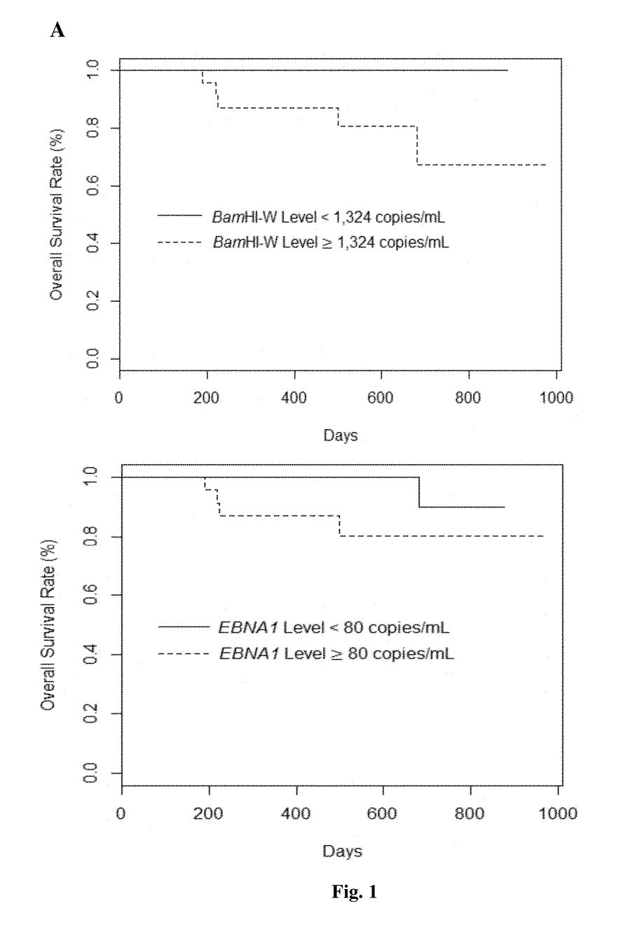

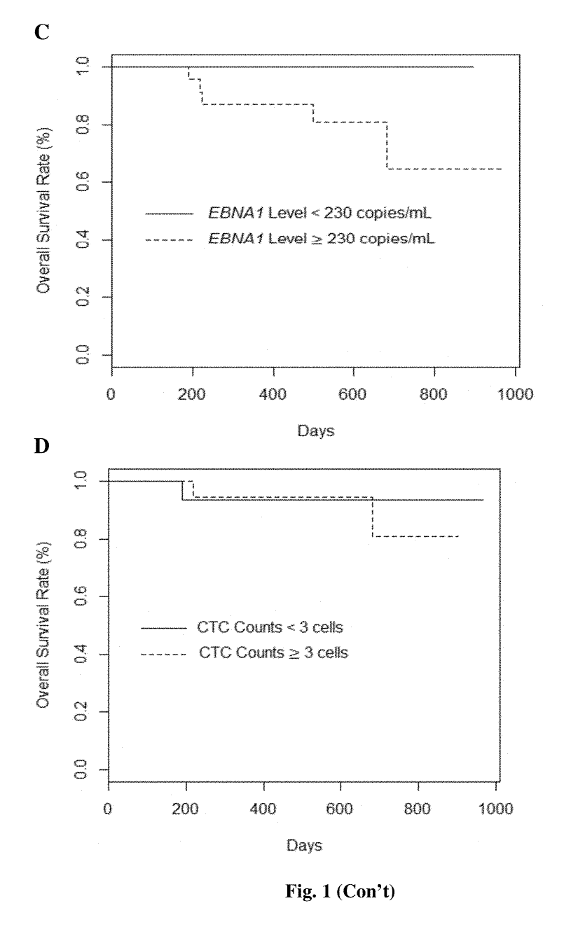

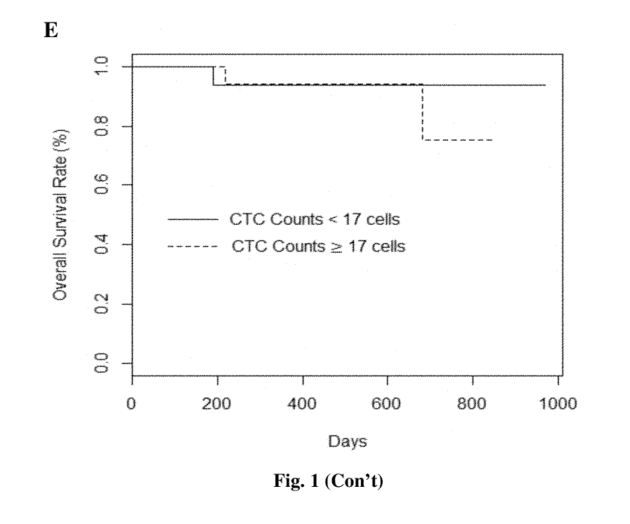

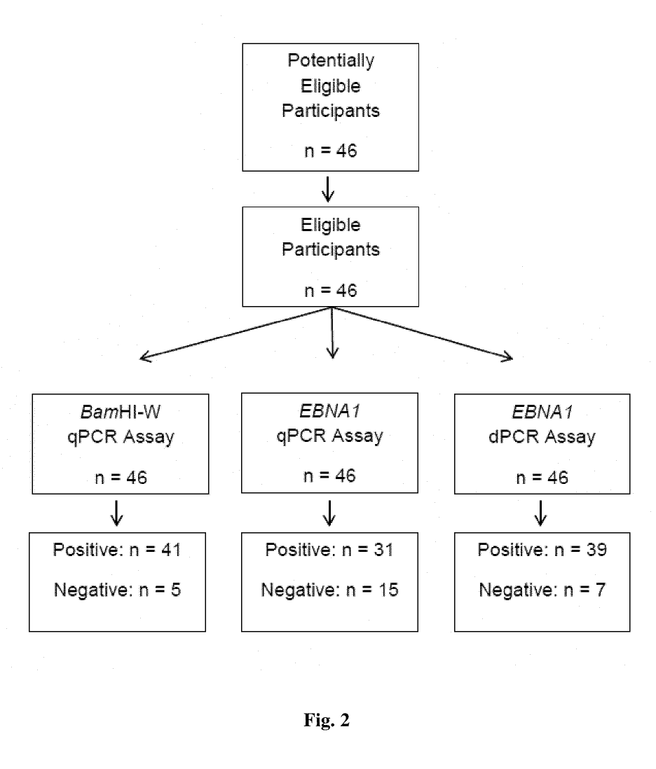

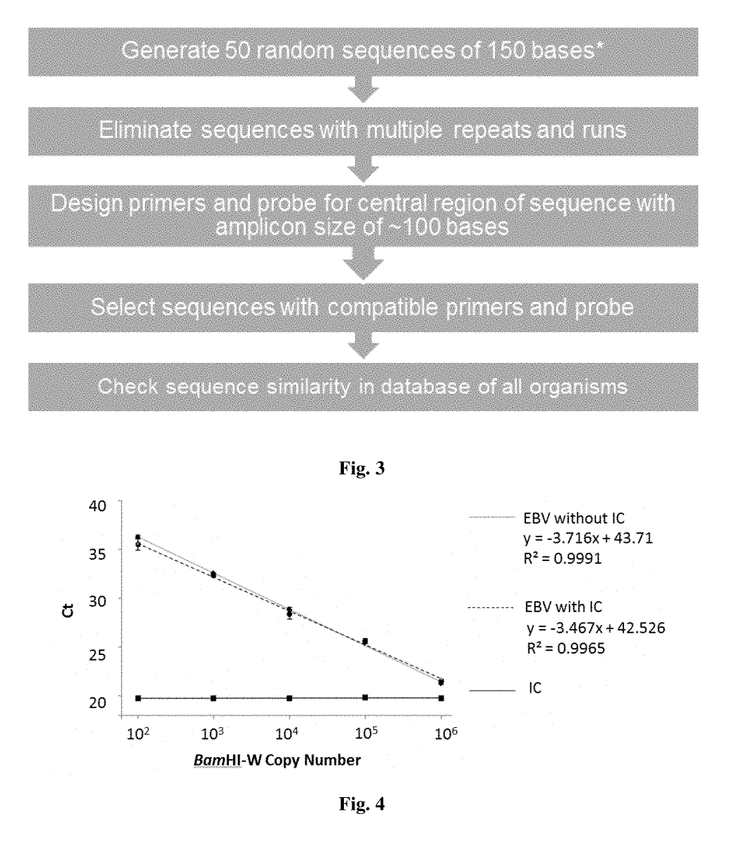

Survival analysis demonstrated that there was a stronger correlation between EBV cfDNA and overall survival, as compared to that between CTC counts and overall survival. All three EBV cfDNA techniques showed prognostic value on survival analysis: BamHI-W qPCR, EBNA1-dPCR and EBNA1-qPCR assays yielded corresponding p-values of 0.03, 0.02 and 0.0002 by log-rank testing respectively, whereas canonical CTC and potential CTC counts were not associated with overall survival (p=0.66 and 0.13 respectively). Kaplan-Meier plots are also shown for dichotomized biomarker variables (FIG. 1).

### EXAMPLE 6

### Derivation of Internal Control for the Amplification of EBV

TABLE 8
Derivation of Internal Control (IC) for the amplification of EBV

| | Master Mix | | | | | | | | | |
|---|---|---|---|---|---|---|---|---|---|---|
| | 1 | | 2 | | 3 | | 4 | | 5 | |
| EBV Primers/Probe | + | | + | | + | | + | | − | |
| IC Primers/Probe | + | | + | | − | | − | | + | |
| IC Oligo | + | | − | | − | | + | | + | |
| Mean Ct* | EBV¹ | IC² | EBV¹ | IC² | EBV¹ | IC² | EBV¹ | IC² | EBV¹ | IC² |
| C666-1 | 24.61 | 22.21 | 24.81 | U | 24.63 | U | 25.29 | U | U | 22.39 |
| RKO | U | 22.13 | U | U | U | U | U | U | U | 22.43 |
| Healthy Donor# | U | 22.16 | U | U | U | U | U | U | U | 22.43 |
| NTC | U | 22.13 | U | U | U | U | U | U | U | 22.39 |

*Triplicate
#Buffy coat DNA
¹FAM signal
²VIC signal
U: Undetermined signal
EBV: Epstein-Barr Virus
IC: Internal Control

The nucleic acid sequence of the Internal Control (IC) selected is

(SEQ ID NO.: 80)
TTAGCAGCGACGAAGATCATGCGCTCACGCTCTCGGTGTCCTCATTCATC
AGTTATTCACAACGCTATGCTGTAACTCGACCTGACAAGACTGTACCTAT
GAGAAGGCACTTGCTACCTTATGCAAGCGTCAGCCCGCGGTATCGCTTG
G.

The sequences of the primers and probes used to amplify the IC are: forward Primer:

(SEQ ID NO.: 81)
5′-CGCTCTCGGTGTCCTCATTC-3′,
Reverse Primer:
(SEQ ID NO.: 82)
5′-GGCTGACGCTTGCATAAGGT-3′,
and
Probe:
(SEQ ID NO.: 83)
5′-VIC-CACAACGCTATGCTGTAACTCGACCTGAC-TAMRA-3′.

Concentration of IC primers and probe is 400 nM and 100 nM respectively (same as BamHI-W primers and probe). As shown in Table 8, Master mix 1 contains IC and all the components of the duplex assay. Therefore, both FAM and VIC signals should be seen in C666-1, an EBV-positive cell line. RKO, buffy coat of healthy donor and no-template control are EBV-negative, thus, will only emit VIC signal. Master mix 2, 3, 4 contain EBV primers and probe without IC Primers and probes or IC sequence or both. So VIC signal should not be present in all samples. This is to test if any component of the IC assay interfere the EBV assay. The last master mix only contains the IC sequence, primers and probe. VIC signal should be positive whereas FAM signal should be negative for all samples. And the results are as expected. The mean Ct values of both FAM and VIC signals in different setups are about the same. The duplex assay is then applied on a serial dilution of EBV sample with or without IC. As shown in FIG. 4, the two straight lines are almost overlapping, indicating no interference between EBV and IC assays.

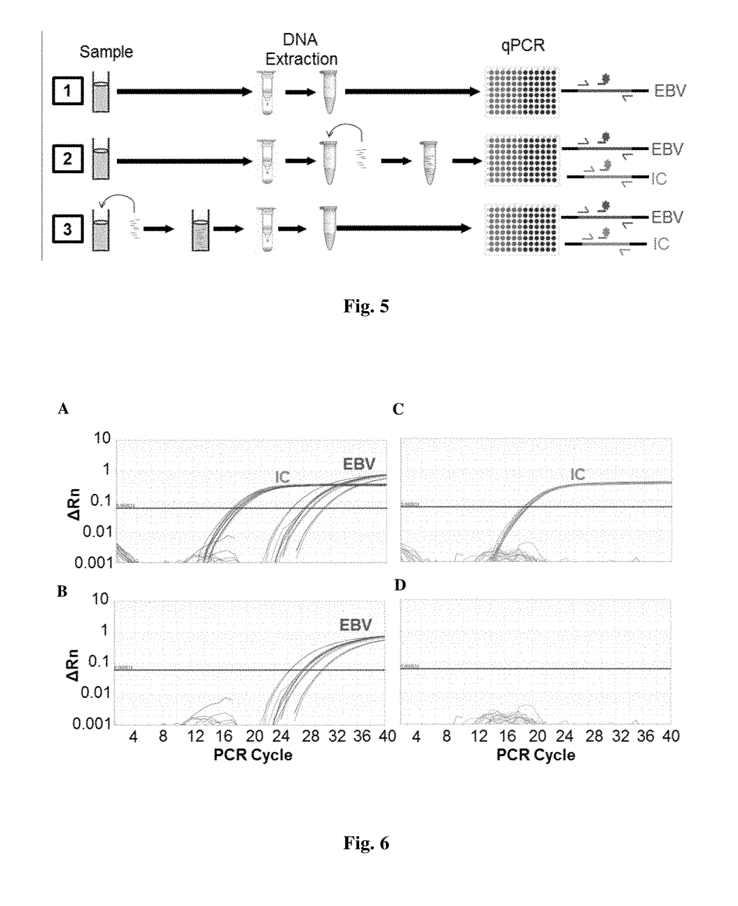

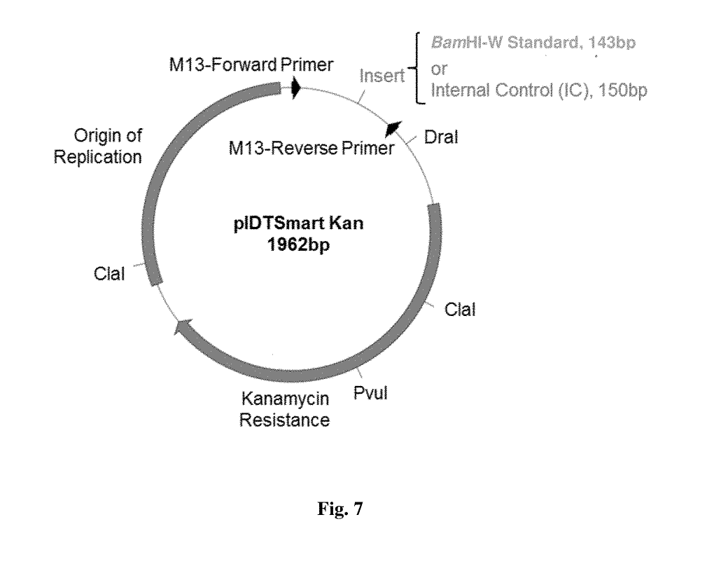

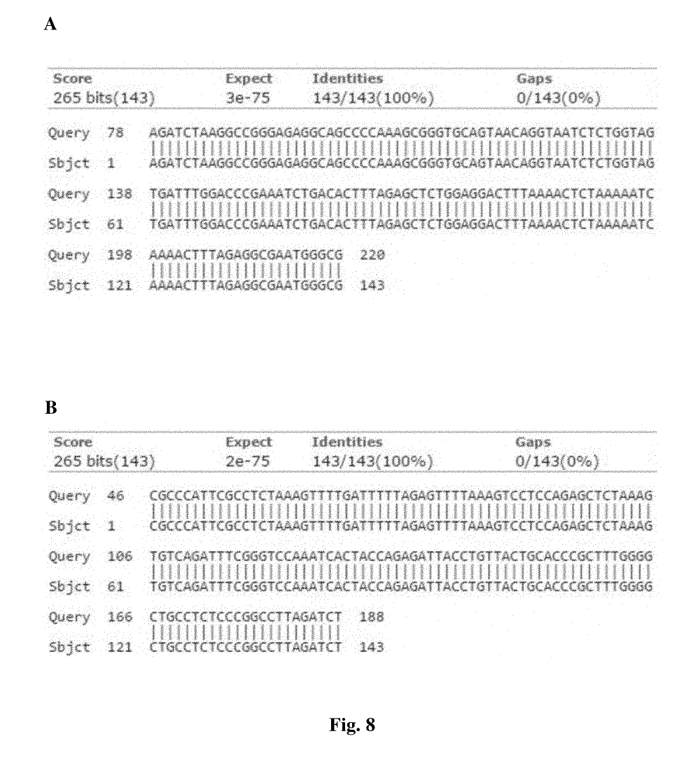

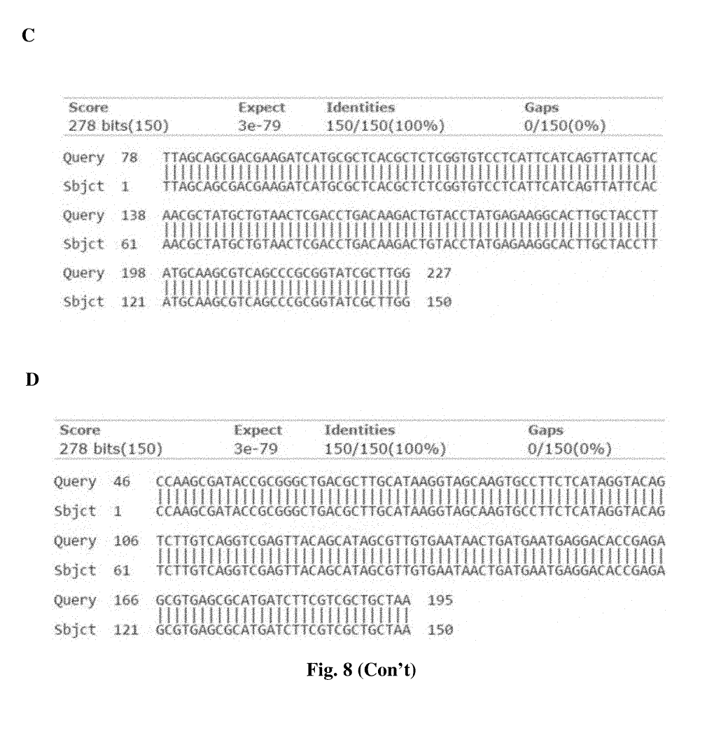

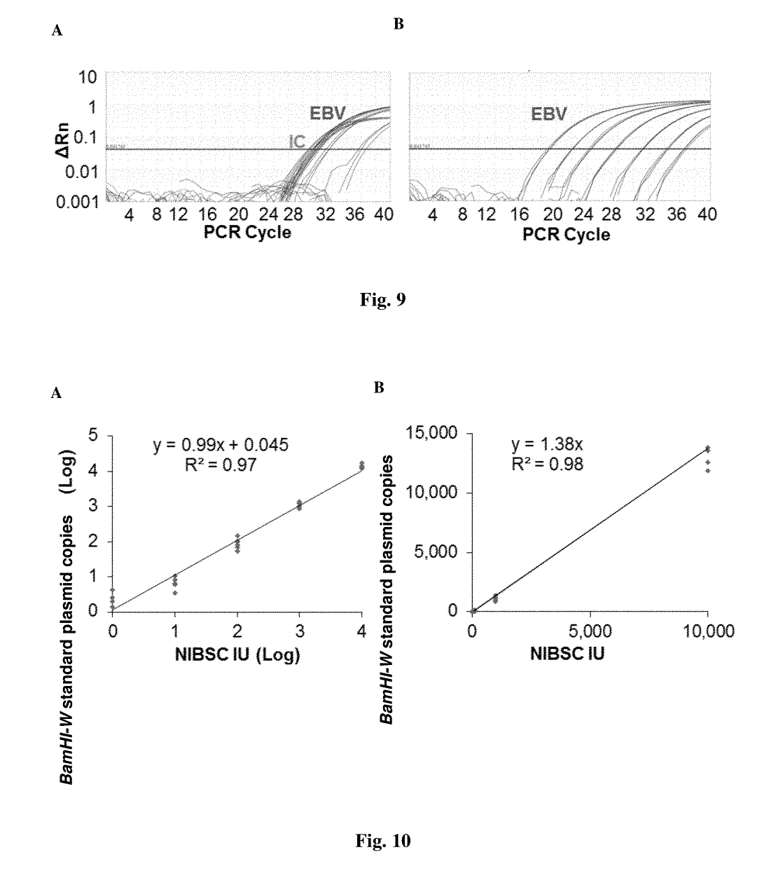

The duplex assay is tested in clinical samples, including 5 EBV-positive NPC samples, and 5 healthy donor's samples. As illustrated in FIG. 5 and shown in Table 9, in setup 1, no IC is spiked, thus, VIC signal should be negative in all samples. In setup 2 and 3, IC is spiked to extracted DNA or samples respectively, so VIC signal will be seen in both setups. And once again, the results are as expected. The small SD of mean Ct in each NPC sample indicates consistent measurement of EBV regardless whether IC is spiked or which step it is spiked. If can be noticed that the mean Ct of IC in setup 3 is smaller than the one in setup 2. This is because the quantity of IC spiked in setup 3 was 10 times higher than the one in setup 2. This was done to accommodate the possible DNA loss during extraction process, however, as shown in Table 9, most of the IC was recovered very well.

TABLE 9
Impact of IC assay on EBV quantification

| | | NPC Pre-Treatment | | | | | Healthy Donor | | | | |
|---|---|---|---|---|---|---|---|---|---|---|---|
| | Mean | Samples | | | | | Samples | | | | |
| Setup | Ct* | 1 | 2 | 3 | 4 | 5 | 6 | 7 | 8 | 9 | 10 |
| 1 | EBV¹ | 30.30 | 25.47 | 27.83 | 27.18 | 28.59 | U | U | U | U | U |
| | IC² | U | U | U | U | U | U | U | U | U | U |
| 2 | EBV¹ | 30.65 | 25.81 | 28.02 | 27.41 | 28.55 | U | U | U | U | U |
| | IC² | 20.72 | 20.76 | 20.73 | 20.81 | 20.80 | 20.80 | 20.80 | 20.88 | 20.9 | 20.86 |
| 3 | EBV¹ | 30.98 | 26.35 | 28.29 | 28.19 | 29.08 | U | U | U | U | U |
| | IC² | 17.73 | 18.15 | 17.12 | 18.24 | 17.39 | 18.50 | 18.57 | 17.90 | 18.04 | 18.35 |

*Triplicate
¹FAM signal
²VIC signal
U: Undetermined signal
EBV: Epstein-Barr Virus
IC: Internal Control

The IC plasmids were spiked to plasma samples and underwent DNA extraction whereas EBV standard plasmids were added directly to the PCR reactions. As shown in FIG. 6, successful amplification of both EBV standard and IC indicates plasmids can be amplified as whole or fragments. As shown in Table 10, in 6 clinical samples, IC were detected, confirming true negative detection of EBV in sample 2 and 6. Sample 6 was indeed negative control, from healthy donor whereas sample 5 was positive control, from an NPC sample with known EBV positive result in plasma. Samples 1 to 4 were also derived from NPC patients but EBV levels in plasma were unknown.

TABLE 10
Plasmid validation in clinical samples

| | Samples | | | | | |
|---|---|---|---|---|---|---|
| | 1 | 2 | 3 | 4 | 5 | 6 |
| Mean EBV Standard | 84 | 0 | 325 | 3 | 239 | 0 |
| Plasmid Copies* | | | | | | |
| Standard Deviation | 13 | N.A. | 26 | 1 | 39 | N.A. |
| IC Signal | + | + | + | + | + | + |

*Triplicate

### EXAMPLE 7

### Derivation of a new Standard for Quantifying EBV based on the Number of Copies of BamHI-W

Despite being a powerful tool in NPC prognosis, the quantification of EBV cfDNA faces challenges of standardization. The NIBSC standards, which are derived from whole EBV produced by B95-8 cells provide a consensus estimate of EBV IU, but are not ideal for standardization of BamHI-W copy number. In addition, the NIBSC spike-in standards do not truly represent the NPC plasma samples. Naturally occurring cfDNA has a size of less than 181 bp in NPC plasma whereas DNA obtained from NIBSC was genomic DNA with a size of 170 kb. The differences in DNA size influence the choice of DNA extraction kit, which in turn has meaningful impact on DNA recovery, and subsequently DNA quantification.

To solve these problems, a new standard for the quantification of EBV cfDNA is developed in the present study.

The sequence of BamHI-W standard was incorporated as an insert in plasmid which was propagated in competent bacteria cells (such as E. coli). Single colony was picked and further grown in Lysogeny Broth (LB) for scale-up production of the plasmids. Bacteria were harvested and plasmids were extracted using the QIAprep Spin Miniprep Kit (Qiagen) and quantified using Quantus Fluorometer (Promega). Sequence of BamHI-W in the newly produced plasmids was confirmed by Sanger Sequencing.

BamHI-W standard plasmids were prepared with known copy number (preferably 10-time serial dilution) then added directly to the PCR well. Standard curve was plotted based on the Ct values obtained from the BamHI-W standard plasmids, which were used to calculate sample's BamHI-W copy number. In the conversion factor experiment, NIBSC standards were set at standard curve to which BamHI-W standard plasmids were calibrated.

### EXAMPLE 8

### Detection of Target Nucleic Acid Sequence of Mycobacterium Tuberculosis

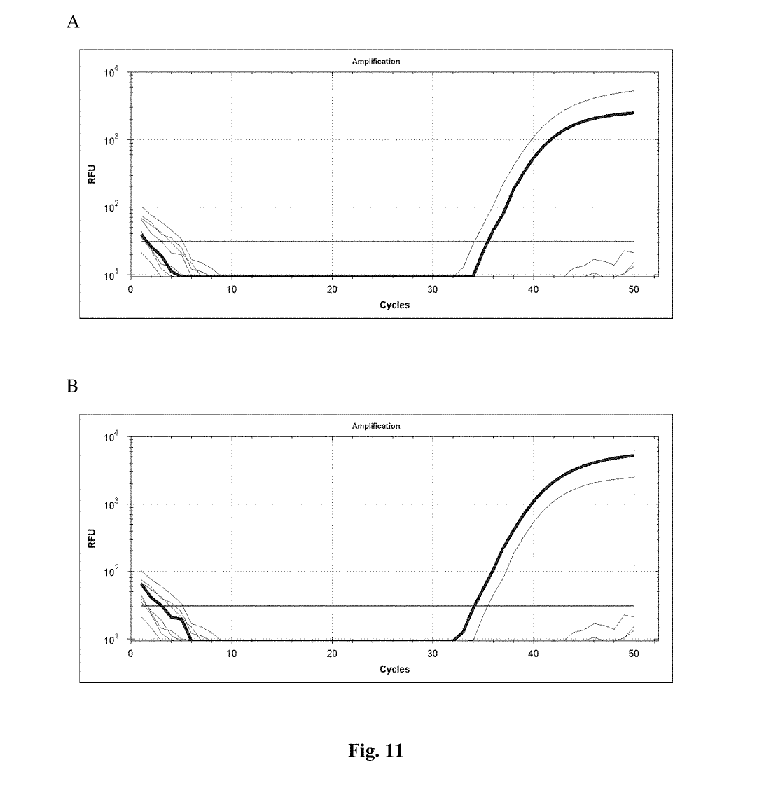

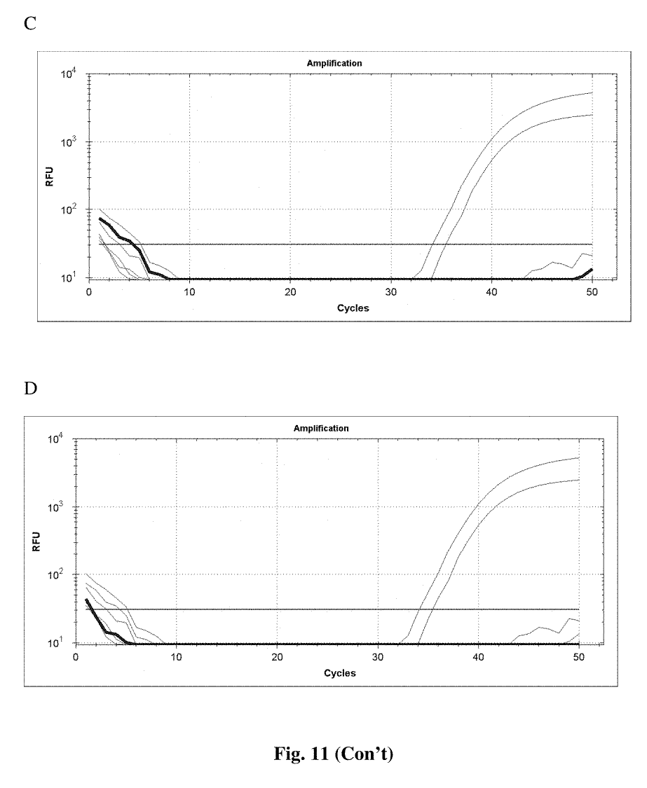

Two targets sequences were tested for the amplification of nucleic acid sequence of Mycobacterium tuberculosis in samples collected from a patient known to have tuberculosis and a healthy subject as control. The target sequences are: (1) nucleotide sequences from nucleotide position 1542511 to nucleotide position 1542349; and (2) nucleotide sequences from nucleotide position 1542328 to nucleotide position 1542215 of the genomic sequence of Mycobacteriaum tuberculosis. The primers and probes used for the amplification of target sequence (1) are: forward primer 5′-GGCTGTGGGTAGCAGACC-3′ (SEQ ID NO.: 74), reverse primer 5′-CGGGTCCAGATGGCTTGC-3′ (SEQ ID NO.: 76) and probe 5′-FAM-TGTCGACCTGGGCAGGGTTCG-TAMRA-3′ (SEQ ID NO.: 84). The primers and probes used for the amplification of target sequence (2) are: forward primer 5′-ACCTGAAAGACGTTATCCACCAT-3′(SEQ ID NO.: 75), reverse primer 5′-CGGCTAGTGCATTGTCATAGGA-3′ (SEQ ID NO.: 77), and probe 5′-FAM-TCCGACCGCGCTCCGACCGACG-TAMRA-3′ (SEQ ID NO.: 85). From the PCR amplification results shown in FIG. 11, it is concluded that both target sequences of Mycobacterium tuberculosis are present in the sample from the patient with tuberculocis, but absent in the sample from the healthy subject control.

Discussion

Non-invasive approaches of NPC diagnosis have been available for the past decade via the detection of immunoglobulin A antibody against EBV antigens in patients' serum. However, these techniques are inefficient in NPC prognosis and relapse prediction. There is considerable ongoing research into EBV cfDNA in NPC patients for prediction of post-treatment outcomes, and its role in selecting patients for additional adjuvant treatment following definitive therapy.

In the present study, good correlation between EBV cfDNA and clinicopathologic outcomes was consistently demonstrated regardless of approach undertaken: BamHI-W qPCR, EBNA1-qPCR or EBNA1-dPCR assays. Decreased EBV cfDNA levels are commonly observed in almost all patients undergoing treatment, corresponding generally to the short-term post-treatment radiological response, which is commonly a complete or near-complete response. Overall, the results demonstrated that EBV cfDNA yielded better results in comparison with circulating tumor cell (CTC) count as a circulating biomarker for NPC. Regardless of approach, cfDNA showed far stronger correlation with tumor stage, short-term radiological response as well as overall survival, in comparison with CTC counts.

The detection rate of the BamHI-W qPCR assay in the present study was 89%. In comparison with clinically validated assays, the BamHI-W qPCR assay demonstrated better performance. The detection rate of the CE-IVD EBNA1-qPCR assay reported in this study was 67%, despite its claimed clinical sensitivity of 100%, based on 80 EBV-positive samples. Moreover, EBV positive cases reported by the BamHI-W qPCR assay were matched with the ones reported by the SGH assay, which had clinical sensitivity of 79%.

In the present study, by targeting the multiple-repeat BamHI-W fragments, the BamHI-W qPCR assay yielded the highest detection rate in NPC pre-treatment samples. It also yielded the highest sensitivity in measurement of NIBSC spike-in standards despite the possible DNA losses due to the DNA extraction method potentially not optimized to genomic DNA. On the other hand, regardless of being different in fundamental techniques of quantification and EBV targets, BamHI-W qPCR and EBNA1-dPCR assays were strongly correlated in the measurement of EBV levels in pre-treatment samples. This correlation could possibly be aided by the same extraction process from which the cfDNA used in BamHI-W qPCR and EBNA1-dPCR assays was extracted. Altogether, in our interpretation, the BamHI-W qPCR and EBNA1-dPCR assays are more likely to quantify the true values of EBV cfDNA level in pre-treatment samples of NPC patients.

The evidence of EBV cfDNA existing in the form of short and freely-floating fragments in the plasma had led to a conclusion that they were released from apoptotic NPC cells. In other words, the NPC cells releasing EBV cfDNA lysed before they had the chance to enter the bloodstream. This phenomenon could explain the non-correlation between NPC CTC counts and EBV cfDNA levels measured by various assays.

The results of the present study demonstrated that by targeting the multiple-repeat BamHI-W, higher detection rate and sensitivity were achieved.

Further, plasma sample of NPC patients contains both human cfDNA and EBV cfDNA. The two major challenges in detection of EBV cfDNA, and in general, microorganism cfDNA in clinical samples are the degradation of EBV cfDNA and the abundant presence of human genomic DNA which hinders the signals from EBV cfDNA. In order to overcome these challenges, a target region (BamHI-W region) in EBV that is preserved and present in high copy number was selected, in particular the region within the CpG island and near to the 5′ end of CpG island was selected for the following reasons: 1. BamHI-W region occurs in multiple repeats per EBV genome, making detection and quantification more feasible, especially in sample with low EBV copy number and/or limited input volume; 2. The region is near to the 5′ location which would allow for preferential preservation during exonuclease III degradation of the EBV dsDNA; and 3. cfDNA residing within CpG islands is more stable and thus less susceptible to degradation and more likely to be detected amongst the presence of human cfDNA.

In the other example, Mycobacterium tuberculosis, plasma sample was selected from a tuberculosis patient because there were bacterium and human cfDNA present in the plasma and the genome of Mycobacterium is rich in GC content. Our analysis showed CpG islands cover 99.9% of the Mycobacterium tuberculosis genome. cfDNA was extracted from the tuberculosis plasma sample and undergone whole genome sequencing (WGS). All reads were mapped to Mycobacterium tuberculosis (Reference genome M.TB H37Rv) and aligned to the CpG islands data mentioned earlier. The results showed all reads belong to CpG islands on the Mycobacterium tuberculosis genome. In order words, highly fragmented and rare Mycobacterium tuberculosis cfDNA within CpG islands can be sequenced despite the abundant presence of human cfDNA in plasma sample. These results imply the advantage of designing cfDNA assay targeting CpG islands in microorganisms.

Materials and Methods

Clinical Samples

The study was approved by the Centralised Institutional Review Board, SingHealth (Reference number: 2013/354/B) and all methods were carried out in accordance with the approved guidelines. A total of 46 NPC patients, all of Asian ethnicity, who provided informed written consent, were recruited into the study between June 2013 and October 2014 (Table 1). 20 mL of blood was collected in EDTA tube (BD Biosciences) at baseline and one month after treatment. All stage-I and most of stage-II patients received only radiotherapy whereas most patients from stage III and IV received combined chemo-radiotherapy. Only 3 patients received adjuvant chemotherapy. A total of 28 matched serial samples, pre- and post-treatment, were collected. The post-treatment radiological response of all patients was based on their first magnetic resonance imaging/computed tomography scan after treatment (Table 5). The median follow-up was 18.7 months.

Participating Laboratories and Clinic

Institute of Bioengineering and Nanotechnology (IBN) served as the centralised laboratory of the study. Blood samples were collected from consenting NPC patients at National Cancer Centre Singapore, and sent to IBN within the same day of their visits within 4 hours. For each sample, whole blood was used for immediate CTC enumeration, and plasma was obtained, assigned blinded IDs and stored at −80° C. until further use. Each plasma assay had its individually optimized volumes. 250 μL of frozen plasma was distributed to Singapore General Hospital (SGH) where cfDNA extraction and quantification was performed using the Sentosa® SA EBV Quantitative PCR Test (Vela Diagnostics) following manufacturer's requirements. At IBN, 1 mL of thawed plasma was used for cfDNA extraction of which half was quantified by the in-house BamHI-W assay. The other half of the extracted cfDNA was sent to JN Medsys where cfDNA quantification was conducted using the Clarity™ Digital PCR System (JN Medsys).

BamHI-W qPCR Assay

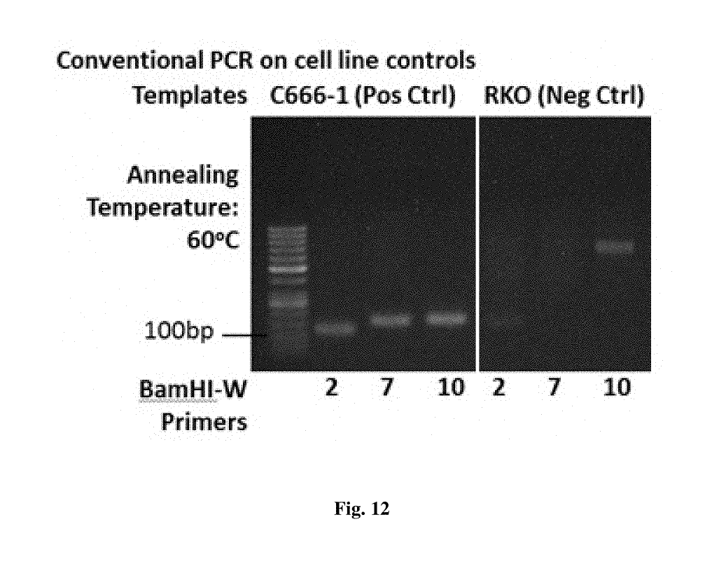

50 μL of cfDNA was extracted from 1 mL of thawed plasma using the QlAamp Circulating Nucleic Acid Kit (Qiagen). The BamHI-W7 primers (Sigma Aldrich) and dual-labelled BamHI-W7 hydrolysis probe (Life Technologies) were designed for the amplification of a 143-bp region of BamHI-W. Each 20-μL reaction consisted of 1× Taqman® Fast Advanced Master Mix (Life Technologies), 400 nM BamHI-W7 primers (sense 5′- AGATCTAAGGCCGGGAGAGG-3′ (SEQ ID NO.: 2) and antisense 5′-CGCCCATTCGCCTCTAAAGT-3′) (SEQ ID NO.: 3), 100 nM BamHI-W7 probe (5′-(6-FAM)CTCTGGTAGTGATTTGGACCCGAAATCTG(TAMRA)-3′) (SEQ ID NO.:4) and 2 μL of DNA template, which was equivalent to 40 μl of plasma. Standard calibrators for BamHI-W were generated with 8 dilutions of DNA derived from EBV-immortalised cell lines ranging from 1 to 10⁷ BamHI-W copies per reaction. qPCR was performed using the ViiA™7 Real-time PCR System (Life Technologies). Each run included patients' cfDNA, standard calibrators, EBV-positive, -negative and no-template controls (NTCs). The reactions were run at 50° C. for 2 min, followed by 95° C. for 20 sec to activate Uracil N-Glycosylase (UNG) and AmpliTaq® Fast DNA Polymerase, respectively. Subsequently, the reactions underwent 40 two-step cycles of denaturation and annealing at 95° C. for 1 sec, and 60° C. for 20 sec, respectively. The BamHI-W copy number was automatically calculated from ViiA™7 software based on the BamHI-W standard calibrator of each run, with R²=0.99, qPCR efficiency=98-100%, m=(−3.315)−(−3.368). Initial optimization of the BamHI-W assay was conducted by conventional PCR using EBV-positive C666-1 DNA (see FIG. 12). BamHI-W specificity for healthy controls has been previously determined to be high and testing of 30 healthy donors also showed no signal.

EBNA1-qPCR Assay

The Sentosa® SA EBV Quantitative PCR Test (Vela Diagnostics) was applied for quantification of EBV cfDNA with the aid of the integrated Sentosa® SX101 (Vela Diagnostics) and Rotor-Gene® Q MDx 5-plex HRM (Qiagen) instruments. 60 μL of DNA was automatically extracted from 200 μL of plasma using the Sentosa® SX Virus Total Nucleic Acid Kit v2.0 (Vela Diagnostics). 10 μL of purified DNA, equivalent to 33 μL of plasma was used for each reaction. The PCR master mix contained reagents and enzymes for the amplification of a 79-bp fragment of EBNA1, as well as a second set of primers/probes designed to detect EC3, a control for PCR inhibition and cfDNA extraction. The concentration of EBNA1 was automatically calculated based on the imported standard curve, with R²=0.99, qPCR efficiency=98%, m=(−3.367). The clinical sensitivity and specificity of the assay was reported as 100% and 98.8% respectively.

EBNA1-d PCR Assay

The Clarity™ Digital PCR System (JN Medsys) was used. The assay was designed to amplify a 118-bp fragment of EBNA1. Each 15-μL reaction consisted of 1× FastStart Essential DNA Probes Master (Roche), 200 nM EBNA1 primers (sense 5′-TCATCATCATCCGGGTCTCC-3′ (SEQ ID NO.: 86) and antisense 5′-GCTCACCATCTGGGCCAC-3′) (SEQ ID NO.: 87), 200 nM probe (5′-(6-FAM)CCTCCAGGTAGAAGGCCATTTTTCCACCCTGTAG(IABKFQ)-3′) (SEQ ID NO.: 88) (Integrated DNA Technologies), 1× Clarity™ JN Solution (JN Medsys), 0.15 U UNG (Roche) and 3 μL of plasma DNA or controls. The equivalent plasma volume per reaction was 60 μL. Each reaction mix was incubated at 40° C. for 10 min to allow UNG to degrade carry-over PCR products, followed by 95° C. for 10 min for UNG inactivation. The reaction mix was partitioned into approximately 10,000 individual reactions in the Clarity™ Digital PCR tube-strip (JN Medsys). Thereafter, the tube-strips were stabilised for 2 min, sealed with 230 μL sealing fluid and subjected to thermal cycling using the following parameters: 1 cycle at 95° C. for 5 min, 40 cycles at 95° C. for 50 sec and 58° C. for 1.5 min. Afterward, the tube-strips were transferred to the Clarity™ Reader (JN Medsys), which detected and quantified fluorescence signals from all partitions. Absolute copy number of EBNA1 in each reaction was determined by the Clarity™ Software (JN Medsys) after analysis of the ratio of positive partitions (i.e. those that contained amplified products) over the total number of partitions, using Poisson statistics.

Determination of Sensitivity and Specificity of EBV cfDNA Assays

All three EBV cfDNA assays were benchmarked against the EBV qPCR assay routinely performed by the College of American Pathologists (CAP)-certified laboratory in SGH. The clinical sensitivity and clinical specificity of the SGH assay was reported as 79% and 100% respectively, based on 66 untreated nasopharyngeal carcinoma patients and 30 normal volunteers. In addition, sensitivity and specificity of EBV cfDNA assays were benchmarked against the 1^{st} World Health Organization (WHO) International Standards for EBV, code 09/260; from National Institute for Biological Standards and Control (NIBSC). The NIBSC standards and nuclease-free water were spiked into EBV-free plasma to obtain 18 standards of 6 known EBV concentrations, ranging from 0 to 1,000,000 IU/mL. In addition, two aliquots of EBV-free plasma served as blank standards. The protocol of DNA extraction, sample distribution and EBV cfDNA assays of spike-in standards was identical to the one for clinical plasma samples.

Enumeration of NPC CTCs

CTCs from 1 mL of whole blood were captured using the microsieve technology and enumerated with the aid of biomarker characterization. The microsieve technology is a size-based method capable of isolating both epithelial and mesenchymal CTCs, unlike the affinity system, which only captures EpCAM-expressed CTCs. Cell counting, and image analysis were performed subject to sample availability, using the MetaMorph software (Molecular Devices) and manually verified by trained laboratory technicians. Cytokeratin-positive and CD45-negative nucleated cells were classified as canonical CTCs. Other nucleated cells that were negative for both cytokeratin and CD45 biomarkers were defined as potential CTCs. All nucleated cells with CD45-positive were classified as white blood cells.

Statistical Analysis

Correlation study was carried out to correlate EBV levels amongst the NPC circulating biomarkers assays. Logistic ordinal regression modelling was used to evaluate pre-treatment circulating biomarker quantitation relative to the dependent variable of clinical stage. Wilcoxon's signed-rank test with continuity correction (R.3.0.0) was conducted to compare paired pre and post-treatment levels of NPC circulating biomarkers. Correlation was performed using Microsoft Excel and the logistic ordinal regression model was performed using the “orm {rms}” library package in R. Alpha was set to 0.05 throughout. Survival analysis was performed using R 3.0.0 survival package to study survival distributions of continuous pre-treatment levels of NPC circulating biomarkers and overall survival (Table 3), using log-rank testing to determine significance at a threshold of 0.05. 1 patient (Patient-025) was omitted from survival analysis, as the patient sought follow-up elsewhere.

1. A method for detecting and/or quantifying the presence of a target nucleic acid sequence of a microorganism in a sample obtained from a subject, comprising amplifying the target sequence in a CpG island of the nucleic acid of the microorganism, irrespective of the methylation status of the CpG island.

2. The method of claim 1, wherein the target sequence is within the 5′ end of the nucleic acid of the CpG island of the nucleic acid of the microorganism.

3. The method of claim 1, wherein the method is a polymerase chain reaction (PCR); optionally wherein the polymerase chain reaction is selected from the group consisting of quantitative polymerase chain reaction, digital polymerase chain reaction, real-time polymerase chain reaction, and traditional polymerase chain reaction.

4. (canceled)

5. The method of claim 1, wherein the sample obtained from the subject is a blood plasma sample, a blood serum sample or a whole blood sample; optionally wherein the sample obtained from the subject comprises cfDNAs (cell free DNAs).

6. (canceled)

7. The method of claim 1, wherein the microorganism is a virus, a bacterium, a fungus or a parasite;
optionally wherein the virus is Epstein-Barr virus (EBV);
optionally wherein the bacterium is a Mycobacterium; and
optionally wherein the Mycobacterium is Mycobacterium tuberculosis.

8.-10. (canceled)

11. The method of claim 5, wherein the target sequence is in a CpG island of the BamHI-W region of EBV;
optionally wherein the CpG island of the BamHI-W region of EBV is selected from the group consisting of:
(SEQ ID NO.: 6)
CTCCTCTCCAACCTTCGCTCCACCCTAGACCCCAGCTTCTGGCCTCCCCG
GGTCCACCAGGCCAGCCGGAGGGACCCCGGCAGCCCGGGCGAGTCGCCTT
CCCTCTCCCCTGGCCTCTCCTTCCCGCCTCCCACCCGAGCCCCCTCAGCT
TGCCTCCCCACCGGGTCCATCAGGCCGGCCGGAGGGACCCCGGCGGCCCG
GTGTCA,
(SEQ ID NO.: 7)
AGGCCATGCGCGCCCTGTCACCAGGCCTGCCAAAGAGCCAGATCTAAGGC
CGGGAGAGGCAGCCCCAAAGCGGGTGCAGTAACAGGTAATCTCTGGTAGT
GATTTGGACCCGAAATCTGACACTTTAGAGCTCTGGAGGACTTTAAAACT
CTAAAAATCAAAACTTTAGAGGCGAATGGGCGCCATTTTGTCCCCACGCG
CGCATAATGGCGGACCTAGGCCTAAAACCCCCAGGAAGCGGGTCTATGGT
TGGCTGCGCTGCTGCTATCTTTAGAGGGGAAAAGAGGAATAAGCCCCCAG
ACAGGGGAGTGGGCTTGTTTGTGACTTCACCAAAGGTCAGGGCCCAAGGG
GGTTCGCGTTGCTAGGCCACCTTCTCAGTC CAGCGCGTTTACGTAAGC,
(SEQ ID NO.: 8)
CTGGTATAAAGTGGTCCTGCAGCTATTTCTGGTCGCATCAGAGCGCCAGG
AGTCCACACAAATGTAAGAGGGGGTCTTCTACCTCTCCCTAGCCCTCCGC
CCCCTCCAAGGACTCGGCCCAGTTTCTAACTTTTCCCCTTCCCTCCCTCG
TCTTGCCCTGCGCCCGGGGCCACCTTCATCACCGTCGCTGACTCCGCCAT
CCAAGCCTAGGGGAGACCGAAGTGAAGGCCCTGGACCAACCCGGCCCGGG
CCCCCCGGTATCGGGCCAGAGGTAAGTGGACTTTAATTTTTTCTGCTAAG
CCCAACACTCCACCACACCCAGGCACACACTACACACACCCACCCGTCTC
AGGGTCCCCTCGGA,
and
(SEQ ID NO.: 9)
CGAGGAGGCGCCCGGAGTGGGGCCGGTCGGCTGGGCTGGCCGAGCCCGGG
TCTGGGAGGTCTGGGGTGGCGAGCCTGCTGTCTCAGGAGGGGCCTGGCTC
CGCCGGGTGGCCCTGGGGTAAGTCTGGGAGGCAGAGGGTCGGCCTAGGCC
CGGGGAAGTGGAGGGGGATCGCCCGGGTCTCTGTTGGCAGAGTCCGGGCG
ATCCTCTGAGACCCTCCGGGCCCGGACGGTCGCCCTCAGCCCCCCAGACA
GACCCCAGGGTCTCCAGGCAGGGTCCGGCATCTTCAGGGGCAGCAGGCTC
ACCACCACAGGCCCCCCAGACCCGGGTCTCGGCCAGCCGAGCCGACCGGC
CCCGCGCCTGGCGCCTCCTCGGGGCCAGCCGCCGGGGTTGGTTCTGCCCC
TCTCTCTGTCCTTCAGAGGAACCAGGGACCTCGGGCACCCCAGAGCCCCT
CGGGCCCGCCTCCAGGCGCCCTCCTGGTCTCCGCTCCCCTCTGAGCCCCG
TTAAACCCAAAGAATGTCTGAGGGGAGCCACCCTCGG;
optionally wherein the target sequence comprises the sequence selected from the group consisting of:
AGATCTAAGGCCGGGAGAGGCAGCCCCAAAGCGGGTGCAGTAACAGGTAATCTCTGG TAGTGATTTGGACCCGAAATCTGACACTTTAGAGCTCTGGAGGACTTTAAAACTCTAAAA ATCAAAACTTTAGAGGCGAATGGGCG (SEQ ID NO.: 1),
GGAATAAGCCCCCAGACAGGGGAGTGGGCTTGTTTGTGACTTCACCAAAGGTCAGGGC CCAAGGGGGTTCGCGTTGCTAGGCCACCTTCTCAGTCCAGCGCGTTTACGTAA (SEQ ID NO.: 10),
AGGAAGCGGGTCTATGGTTGGCTGCGCTGCTGCTATCTTTAGAGGGGAAAAGAGGAAT AAGCCCCCAGACAGGGGAGTGGGCTTGTTTGTGACTTCACCAAAGGTCAGGGCCCAA GGGGGTTCGCGTTGCTAGGCCACCTTCTCAGTC (SEQ ID NO.:11), a complementary sequence, a fragment, and a variant thereof;
optionally wherein amplifying the target sequence comprises the use of a pair of oligonucleotide primers, wherein the first oligonucleotide primer comprises a sequence selected from the group consisting of: 5′-AGATCTAAGGCCGGGAGAGG-3′ (SEQ ID NO.:2), 5′-GGAATAAGCCCCCAGACAGG-3′ (SEQ ID NO.:12), 5′-AGGAAGCGGGTCTATGGTTG-3′ (SEQ ID NO.:13), a fragment and a variant thereof, and
the second oligonucleotide primer comprises a sequence selected from the group consisting of 5′-CGCCCATTCGCCTCTAAAGT-3′ (SEQ ID NO.:3), 5′-TTACGTAAACGCGCTGGACT-3′ (SEQ ID NO.:14), 5′-GACTGAGAAGGTGGCCTAGC-3′ (SEQ ID NO.:15), a fragment and a variant thereof.

12.-14. (canceled)

15. The method of claim 5, wherein amplifying the target sequence further comprises the use of a probe capable of binding to the target sequence;
optionally wherein the probe comprises a sequence selected from the group consisting of 5′-CTCTGGTAGTGATTTGGACCCGAAATCTG-3′ (SEQ ID NO.: 16), 5′- CCACCTTCTCAGTCCAGCGCGTTT-3′ (SEQ ID NO.: 17), 5′- GTGACTTCACCAAAGGTCAGGGCCC-3′ (SEQ ID NO.: 18), 5′- GGTGGTAAGCGGTTCACCTTCAGGG-3′ (SEQ ID NO.: 19), a fragment and a variant thereof.

16. (canceled)

17. The method of claim 5, further comprising calculating the copy number of BamHI-W region in the amplified product, wherein the copy number is calculated using the following formula:
$$Copy Number of BamHI - W = \frac{DNA Quantity ( ng ) \times Avogradro \text{’} s Number}{88390.29 ( Da ) \times {10}^{9}}$$

18. The method of claim 5, wherein the method is capable of detecting the lowest concentration of EBV of 100 IU (International Unit)/ml of sample, or 10 IU/ml of sample, or 1 IU/ml of sample.

19. A method for detecting and/or quantifying the presence of a target nucleic acid sequence of Epstein-Barr virus (EBV) in a sample obtained from a subject, comprising amplifying a target sequence in the BamHI-W region of EBV, wherein the target sequence comprises the sequence of
(SEQ ID NO.: 1)
AGATCTAAGGCCGGGAGAGGCAGCCCCAAAGCGGGTGCAGTAACAGGTAA
TCTCTGGTAGTGATTTGGACCCGAAATCTGACACTTTAGAGCTCTGGAGG
ACTTTAAAACTCTAAAAATCAAAACTTTAGAGGCGAATGGGCG,
wherein amplifying the target sequence comprises the use of a pair of oligonucleotide primers and a probe, wherein the first oligonucleotide primer comprises the sequence of 5′-AGATCTAAGGCCGGGAGAGG-3′ (SEQ ID NO.: 2), and the second oligonucleotide primer comprises the sequence of 5′-CGCCCATTCGCCTCTAAAGT-3′ (SEQ ID NO.:3), and wherein the probe comprises the sequence of
(SEQ ID NO.: 4)
5′-(6 FAM)CTCTGGTAGTGATTTGGACCCGAAATCTG(TAMRA)-3′,
and wherein the method is a quantitative polymerase chain reaction (qPCR).

20. The method of claim 5, wherein the method further comprises amplifying a control comprising a target sequence of TTAGCAGCGACGAAGATCATGCGCTCACGCTCTCGGTGTCCTCATTCATCAGTTATTCA CAACGCTATGCTGTAACTCGACCTGACAAGACTGTACCTATGAGAAGGCACTTGCTACC TTATGCAAGCGTCAGCCCGCGGTATCGCTTGG (SEQ ID NO.: 80), a complementary sequence, a fragment or a variant thereof;
optionally wherein amplifying the control comprises the use of a pair of oligonucleotide primers, wherein the first oligonucleotide primer comprises the sequence of 5′-CGCTCTCGGTGTCCTCATTC-3′ (SEQ ID NO.: 81), a complementary sequence, a fragment or a variant thereof, and the second oligonucleotide primer comprises the sequence of 5′-GGCTGACGCTTGCATAAGGT-3′ (SEQ ID NO.: 82), a complementary sequence, a fragment or a variant thereof; and,
optionally wherein amplifying the control further comprises the use of a probe capable of binding to the target sequence of the control, wherein the probe comprises the sequence of 5′-VIC-CACAACGCTATGCTGTAACTCGACCTGAC-TAMRA-3′ (SEQ ID NO.: 83).

21.-24. (canceled)

25. A method of detecting a disease associated with microorganism infection, or risk of developing a disease associated with microorganism infection in a subject, comprising detecting and/or quantifying the presence of a nucleic acid sequence of the microorganism using the method of claim 1 in a sample obtained from the subject, wherein the presence of the nucleic acid sequence of the microorganism in the sample indicates that the subject has a disease associated with microorganism infection or is at risk of developing a disease associated with microorganism infection;
optionally wherein the disease associated with microorganism infection is selected from the group consisting of Alzheimer's disease, amyotrophic lateral sclerosis, anorexia nervosa, anxiety disorder, asthma, atherosclerosis, autoimmune diseases, cancers, chronic obstructive pulmonary disease, Crohn's disease, coronary heart disease, dementia, diabetes mellitus type 1, diabetes mellitus type 2, dilated cardiomyopathy, epilepsy, Guillain-Barré syndrome, irritable bowel syndrome, lupus, multiple sclerosis, myocardial infarction, Parkinson's disease, psoriasis, rheumatoid arthritis, sarcoidosis, schizophrenia, stroke, thromboangiitis obliterans, tourette syndrome and vasculitis;
optionally wherein the disease associated with microorganism infection is cancer;
optionally wherein the cancer is EBV-associated cancer;
optionally wherein the EBV-associated cancer is selected from the group consisting of nasopharyngeal carcinoma (NPC), gastric cancer, Hodgkin's lymphoma and Burkitt's lymphoma; and
optionally wherein the EBV-associated cancer is NPC.

26. A method of detecting and treating a disease associated with microorganism infection, comprising:
(i) detecting and/or quantifying the presence of a nucleic acid sequence of the microorganism using the method of claim 1 in a sample obtained from the subject, wherein the presence of the nucleic acid sequence of the microorganism in the sample indicates that the subject has a disease associated with microorganism;
(ii) administering to the subject a medicament suitable for the treatment of the disease associated with the microorganism,
optionally wherein the disease associated with microorganism infection is selected from the group consisting of Alzheimer's disease, amyotrophic lateral sclerosis, anorexia nervosa, anxiety disorder, asthma, atherosclerosis, autoimmune diseases, cancers, chronic obstructive pulmonary disease, Crohn's disease, coronary heart disease, dementia, diabetes mellitus type 1, diabetes mellitus type 2, dilated cardiomyopathy, epilepsy, Guillain-Barré syndrome, irritable bowel syndrome, lupus, multiple sclerosis, myocardial infarction, Parkinson's disease, psoriasis, rheumatoid arthritis, sarcoidosis, schizophrenia, stroke, thromboangiitis obliterans, tourette syndrome and vasculitis;
optionally wherein the disease associated with microorganism infection is cancer;
optionally wherein the cancer is EBV-associated cancer;
optionally wherein the EBV-associated cancer is selected from the group consisting of nasopharyngeal carcinoma (NPC), gastric cancer, Hodgkin's lymphoma and Burkitt's lymphoma; and
optionally wherein the EBV-associated cancer is NPC.

27. A method of predicting the treatment outcome of a disease associated with microorganism infection in a patient, comprising:
(i) quantifying the nucleic acid sequence of the microorganism in a sample collected from the patient before treatment or before a treatment step, and quantifying the nucleic acid sequence of the microorganism in a sample collected from the same patient after treatment or after a treatment step;
(ii) comparing the amount of the nucleic acid sequence of the microorganism in the sample before and after treatment or a treatment step, wherein a decrease in the amount of the nucleic acid sequence of the microorganism in the sample after treatment or a treatment step indicates that treatment outcome of the disease associated with microorganism infection in the patient is positive,
wherein the quantifying of the nucleic acid sequence of the microorganism in the sample is performed according to the method of claim 1,
optionally wherein the disease associated with microorganism infection is selected from the group consisting of Alzheimer's disease, amyotrophic lateral sclerosis, anorexia nervosa, anxiety disorder, asthma, atherosclerosis, autoimmune diseases, cancers, chronic obstructive pulmonary disease, Crohn's disease, coronary heart disease, dementia, diabetes mellitus type 1, diabetes mellitus type 2, dilated cardiomyopathy, epilepsy, Guillain-Barré syndrome, irritable bowel syndrome, lupus, multiple sclerosis, myocardial infarction, Parkinson's disease, psoriasis, rheumatoid arthritis, sarcoidosis, schizophrenia, stroke, thromboangiitis obliterans, tourette syndrome and vasculitis;
optionally wherein the disease associated with microorganism infection is cancer;
optionally wherein the cancer is EBV-associated cancer;
optionally wherein the EBV-associated cancer is selected from the group consisting of nasopharyngeal carcinoma (NPC), gastric cancer, Hodgkin's lymphoma and Burkitt's lymphoma; and
optionally wherein the EBV-associated cancer is NPC.

28.-32. (canceled)

33. A kit for detecting and/or quantifying the nucleic acid sequence of a microorganism in a sample obtained from a subject, comprising a pair of oligonucleotide primers specific for the amplification of a target sequence in a CpG island of the nucleic acid of the microorganism.

34. The kit of claim 33, wherein the target sequence is within the 5′end of the CpG island of the nucleic acid of the microorganism.

35. The kit of claim 33, wherein the sample obtained from the subject is a blood plasma sample, a blood serum sample or a whole blood sample; optionally wherein the sample obtained from the subject comprises cfDNAs (cell free DNAs).

36. (canceled)

37. The kit of claim 33, wherein the microorganism is a virus, a bacterium, a fungus or a parasite; optionally wherein the virus is Epstein-Barr virus (EBV).

38.-40. (canceled)

41. The kit of claim 37, wherein the target sequence is in a CpG island of the BamHI-W region of EBV;
optionally wherein the target sequence comprises the sequence selected from the group consisting of:
AGATCTAAGGCCGGGAGAGGCAGCCCCAAAGCGGGTGCAGTAACAGGTAATCTCTGG TAGTGATTTGGACCCGAAATCTGACACTTTAGAGCTCTGGAGGACTTTAAAACTCTAAAA ATCAAAACTTTAGAGGCGAATGGGCG (SEQ ID NO.: 1),
GGAATAAGCCCCCAGACAGGGGAGTGGGCTTGTTTGTGACTTCACCAAAGGTCAGGGC CCAAGGGGGTTCGCGTTGCTAGGCCACCTTCTCAGTCCAGCGCGTTTACGTAA (SEQ ID NO.: 10)
AGGAAGCGGGTCTATGGTTGGCTGCGCTGCTGCTATCTTTAGAGGGGAAAAGAGGAAT AAGCCCCCAGACAGGGGAGTGGGCTTGTTTGTGACTTCACCAAAGGTCAGGGCCCAA GGGGGTTCGCGTTGCTAGGCCACCTTCTCAGTC (SEQ ID NO.:11), a complementary sequence, a fragment and a variant thereof;
optionally wherein the first oligonucleotide primer comprises a sequence selected from the group consisting of: 5′-AGATCTAAGGCCGGGAGAGG-3′ (SEQ ID NO.: 2), 5′-GGAATAAGCCCCCAGACAGG-3′ (SEQ ID NO.:12), 5′-AGGAAGCGGGTCTATGGTTG-3′ (SEQ ID NO.:13), a fragment and a variant thereof, and the second oligonucleotide primer comprises a sequence selected from the group consisting of 5′-CGCCCATTCGCCTCTAAAGT-3′ (SEQ ID NO.:3), 5′- TTACGTAAACGCGCTGGACT-3′ (SEQ ID NO.:14), 5′-GACTGAGAAGGTGGCCTAGC-3′ (SEQ ID NO.:15), a fragment and a variant thereof.

42. and 43. (canceled)

44. The kit of claim 33, further comprises a probe capable of binding to the target sequence; optionally wherein the probe comprises a sequence selected from the group consisting of 5′-CTCTGGTAGTGATTTGGACCCGAAATCTG-3′ (SEQ ID NO.: 16), 5′-CCACCTTCTCAGTCCAGCGCGTTT-3′ (SEQ ID NO.: 17), 5′-GTGACTTCACCAAAGGTCAGGGCCC-3′ (SEQ ID NO.: 18), 5′-GGTGGTAAGCGGTTCACCTTCAGGG-3′ (SEQ ID NO.: 19), a fragment and variant thereof.

45. The kit of claim 44, wherein the probe comprises a sequence selected from the group consisting of 5′-CTCTGGTAGTGATTTGGACCCGAAATCTG-3′ (SEQ ID NO.: 16), 5′- CCACCTTCTCAGTCCAGCGCGTTT-3′ (SEQ ID NO.: 17), 5′-GTGACTTCACCAAAGGTCAGGGCCC-3′ (SEQ ID NO.: 18), 5′-GGTGGTAAGCGGTTCACCTTCAGGG-3′ (SEQ ID NO.: 19), a fragment and variant thereof.


SEQUENCE LISTING (89 sequences)

SEQ ID NO: 1 - DNA, 143 bp, Human herpesvirus 4
agatctaaggccgggagaggcagccccaaagcgggtgcagtaacaggtaatctctggtagtgatttggacccgaaatctgacactttagagctctggaggactttaaaactctaaaaatcaaaactttagaggcgaatgggcg

SEQ ID NO: 2 - DNA, 20 bp, Artificial Sequence
  Synthetic sequence
agatctaaggccgggagagg

SEQ ID NO: 3 - DNA, 20 bp, Artificial Sequence
  Synthetic sequence
cgcccattcgcctctaaagt

SEQ ID NO: 4 - DNA, 29 bp, Artificial Sequence
  Synthetic sequence
  misc_feature (1)..(1): 6-carboxyfluorescein (6-FAM)
  misc_feature (29)..(29): 5-Carboxytetramethylrhodamine (TAMRA)
ctctggtagtgatttggacccgaaatctg

SEQ ID NO: 5 - DNA, 3072 bp, Human herpesvirus 4
ggatccccccaccggcccttctctctgtccccctgctcctctccaaccttcgctccaccctagaccccagcttctggcctccccgggtccaccaggccagccggagggaccccggcagcccgggcgagtcgccttccctctcccctggcctctccttcccgcctcccacccgagccccctcagcttgcctccccaccgggtccatcaggccggccggagggaccccggcggcccggtgtcagtcccccctgcagccgcccagtctctgcctccaggcaagggcgccagcttttctccccccagcctgaggcccagtctcctgtgcactgtctgtaaagtccagcctcccacgcccgtccacggctcccgggcccagcctcgtccacccctccccacggtggacaggccctctgtccacccgggccatccccgcccccctgtgtccaccccagtcccgtccaggggggactttatgtgacccttgggcctggctccccatagactcccatgtaagcctgcctcgagtaggtgcctccagagccccttttgcccccctggcggcccagcccgacccccgggcgcccccaaactttgtccagatgtccaggggtccccgagggtgaggcccagccccctcccgcccctgtccactgccccggtccccccagaagcccccaaaagtagaggctcaggccatgcgcgccctgtcaccaggcctgccaaagagccagatctaaggccgggagaggcagccccaaagcgggtgcagtaacaggtaatctctggtagtgatttggacccgaaatctgacactttagagctctggaggactttaaaactctaaaaatcaaaactttagaggcgaatgggcgccattttgtccccacgcgcgcataatggcggacctaggcctaaaacccccaggaagcgggtctatggttggctgcgctgctgctatctttagaggggaaaagaggaataagcccccagacaggggagtgggcttgtttgtgacttcaccaaaggtcagggcccaagggggttcgcgttgctaggccaccttctcagtccagcgcgtttacgtaagccagacagcagccaattgtcagttctagggagggggaccactgcccctggtataaagtggtcctgcagctatttctggtcgcatcagagcgccaggagtccacacaaatgtaagagggggtcttctacctctccctagccctccgccccctccaaggactcgggcccagtttctaacttttccccttccctccctcgtcttgccctgcgcccggggccaccttcatcaccgtcgctgactccgccatccaagcctaggggagaccgaagtgaaggccctggaccaacccggcccgggccccccggtatcgggccagaggtaagtggactttaattttttctgctaagcccaacactccaccacacccaggcacacactacacacacccacccgtctcagggtcccctcggacagctcctaagaaggcaccggtcgcccagtcctaccagagggggccaagaacccagacgagtccgtagaagggtcctcgtccagcaagaagaggaggtggtaagcggttcaccttcaggggtaagtaacctgacctctccagggctcacataaagggaggcttagtatacatgcttcttgcttttcacaggaacctgggggctagtctgggtgggattaggctgcctcaagttgcatcagccagggcttcatgccctcctcagttccctagtccccgggcttcaggccccctccgtccccgtcctccagagacccgggcttcaggccctgcctctcctgttacccttttagaaccacagcctggacacatgtgccagacgccttggcctctaaggccctcgggtccccctggaccccggcctcagcaaccctgctgctcccctcctgccaccccagcctccccccctccccgtcccccttcgctcctgatcctcccccggtccccagtagggccgcctgcccccctgcacccagtacctgcccctcttggccacgcaccccgggccaggccaccttagacccggccaagccccatccctgaagacccagcggccattctctctggtaacgagcagagaagaagtagaggcccgcggccattgggcccagattgagagaccagtccaggggcccgaggttggagccagcgggcacccgaggtcccagcacccggtccctccggggggcagagacaggcagggccccccggcagctggccccgaggaggcgcccggagtggggccggtcggctgggctggccgagcccgggtctgggaggtctggggtggcgagcctgctgtctcaggaggggcctggctccgccgggtggccctggggtaagtctgggaggcagagggtcggcctaggcccggggaagtggagggggatcgcccgggtctctgttggcagagtccgggcgatcctctgagaccctccgggcccggacggtcgccctcagccccccagacagaccccagggtctccaggcagggtccggcatcttcaggggcagcaggctcaccaccacaggccccccagacccgggtctcggccagccgagccgaccggccccgcgcctggcgcctcctcggggccagccgccggggttggttctgcccctctctctgtccttcagaggaaccagggacctcgggcaccccagagcccctcgggcccgcctccaggcgccctcctggtctccgctcccctctgagccccgttaaacccaaagaatgtctgaggggagccaccctcggggcccaggccccagagtccagaggtcaggggcacctcagggtgcctccccgggtcccaggccagccggagggaccccggcagcccgggcggccccagaggccggttcctcgccccttccccgggcttcagagcccaggatgtcccccagaagggaccctaggcgtcccctctcctcccctccaggcccgagcctctccctcgcggagaggggcctctttgggccctcaagtccagccccaccgagacccgagtggccc

SEQ ID NO: 6 - DNA, 206 bp, Human herpesvirus 4
ctcctctccaaccttcgctccaccctagaccccagcttctggcctccccgggtccaccaggccagccggagggaccccggcagcccgggcgagtcgccttccctctcccctggcctctccttcccgcctcccacccgagccccctcagcttgcctccccaccgggtccatcaggccggccggagggaccccggcggcccggtgtca

SEQ ID NO: 7 - DNA, 398 bp, Human herpesvirus 4
aggccatgcgcgccctgtcaccaggcctgccaaagagccagatctaaggccgggagaggcagccccaaagcgggtgcagtaacaggtaatctctggtagtgatttggacccgaaatctgacactttagagctctggaggactttaaaactctaaaaatcaaaactttagaggcgaatgggcgccattttgtccccacgcgcgcataatggcggacctaggcctaaaacccccaggaagcgggtctatggttggctgcgctgctgctatctttagaggggaaaagaggaataagcccccagacaggggagtgggcttgtttgtgacttcaccaaaggtcagggcccaagggggttcgcgttgctaggccaccttctcagtccagcgcgtttacgtaagc

SEQ ID NO: 8 - DNA, 365 bp, Human herpesvirus 4
ctggtataaagtggtcctgcagctatttctggtcgcatcagagcgccaggagtccacacaaatgtaagagggggtcttctacctctccctagccctccgccccctccaaggactcgggcccagtttctaacttttccccttccctccctcgtcttgccctgcgcccggggccaccttcatcaccgtcgctgactccgccatccaagcctaggggagaccgaagtgaaggccctggaccaacccggcccgggccccccggtatcgggccagaggtaagtggactttaattttttctgctaagcccaacactccaccacacccaggcacacactacacacacccacccgtctcagggtcccctcgga

SEQ ID NO: 9 - DNA, 537 bp, Human herpesvirus 4
cgaggaggcgcccggagtggggccggtcggctgggctggccgagcccgggtctgggaggtctggggtggcgagcctgctgtctcaggaggggcctggctccgccgggtggccctggggtaagtctgggaggcagagggtcggcctaggcccggggaagtggagggggatcgcccgggtctctgttggcagagtccgggcgatcctctgagaccctccgggcccggacggtcgccctcagccccccagacagaccccagggtctccaggcagggtccggcatcttcaggggcagcaggctcaccaccacaggccccccagacccgggtctcggccagccgagccgaccggccccgcgcctggcgcctcctcggggccagccgccggggttggttctgcccctctctctgtccttcagaggaaccagggacctcgggcaccccagagcccctcgggcccgcctccaggcgccctcctggtctccgctcccctctgagccccgttaaacccaaagaatgtctgaggggagccaccctcgg

SEQ ID NO: 10 - DNA, 111 bp, Human herpesvirus 4
ggaataagcccccagacaggggagtgggcttgtttgtgacttcaccaaaggtcagggcccaagggggttcgcgttgctaggccaccttctcagtccagcgcgtttacgtaa

SEQ ID NO: 11 - DNA, 148 bp, Human herpesvirus 4
aggaagcgggtctatggttggctgcgctgctgctatctttagaggggaaaagaggaataagcccccagacaggggagtgggcttgtttgtgacttcaccaaaggtcagggcccaagggggttcgcgttgctaggccaccttctcagtc

SEQ ID NO: 12 - DNA, 20 bp, Artificial Sequence
  Synthetic sequence
ggaataagcccccagacagg

SEQ ID NO: 13 - DNA, 20 bp, Artificial Sequence
  Synthetic sequence
aggaagcgggtctatggttg

SEQ ID NO: 14 - DNA, 20 bp, Artificial Sequence
  Synthetic sequence
ttacgtaaacgcgctggact

SEQ ID NO: 15 - DNA, 20 bp, Artificial Sequence
  Synthetic sequence
gactgagaaggtggcctagc

SEQ ID NO: 16 - DNA, 29 bp, Artificial Sequence
  Synthetic sequence
ctctggtagtgatttggacccgaaatctg

SEQ ID NO: 17 - DNA, 24 bp, Artificial Sequence
  Synthetic sequence
ccaccttctcagtccagcgcgttt

SEQ ID NO: 18 - DNA, 25 bp, Artificial Sequence
  Synthetic sequence
gtgacttcaccaaaggtcagggccc

SEQ ID NO: 19 - DNA, 25 bp, Artificial Sequence
  Synthetic sequence
ggtggtaagcggttcaccttcaggg

SEQ ID NO: 20 - DNA, 150 bp, Mycobacterium tuberculosis
ggtcgccgcgggcaggctcaacccggagcggatcaccgaatccacgatcgcccgccacctgcagcgacccgacattcccgacgttgacctcttcctgcggacctcgggtgagcagcgctccagcaacttcatgctgtggcaggcggccta

SEQ ID NO: 21 - DNA, 150 bp, Mycobacterium tuberculosis
cctcgcaggccgcccacaagtcgcggcggtcatagtcgggccagagcttgtcctggaatatgtattcagcgtaggccgcctgccacagcatgaagttgctggagcgctgctcacccgaggtccgcaggaagaggtcaacgtcgggaatgt

SEQ ID NO: 22 - DNA, 150 bp, Mycobacterium tuberculosis; identical to SEQ ID NO: 20

SEQ ID NO: 23 - DNA, 150 bp, Mycobacterium tuberculosis; identical to SEQ ID NO: 21

SEQ ID NO: 24 - DNA, 108 bp, Mycobacterium tuberculosis
tggtggtcggctcgcggccggtgtcgagatcaccgaaccgcaaagaagtgtggtcgtcttcgacaccgcacccaccgccctgctgcgggtttaccgcgacaagcttcc

SEQ ID NO: 25 - DNA, 108 bp, Mycobacterium tuberculosis
ggaagcttgtcgcggtaaacccgcagcagggcggtgggtgcggtgtcgaagacgaccacacttctttgcggttcggtgatctcgacaccggccgcgagccgaccacca

SEQ ID NO: 26 - DNA, 150 bp, Mycobacterium tuberculosis
gatgtccgggctcaccccggcgtggtcggcggcgaacaacgcgccggtgcggccgaagccggtggcgatctcatcgaagatcagcagcacctcgtaacggcggcagatgtcccgcaggtcgtgcagatagcgcgggtcgtgaaaacgcat

SEQ ID NO: 27 - DNA, 150 bp, Mycobacterium tuberculosis
gcacgccggcgagctggccgcggtggtcgtggagccggtcgtgcagggtgcgggcggtatgcgttttcacgacccgcgctatctgcacgacctgcgggacatctgccgccgttacgaggtgctgctgatcttcgatgagatcgccaccgg

SEQ ID NO: 28 - DNA, 150 bp, Mycobacterium tuberculosis
ggtatggtcgccgccgcgacgccgctgccggtgatcggggtgcccgtaccgctgggcaggctggacgggcttgactccctgctgtcgatcgtgcaaacgccggccgggggtccggtggccacgctccccatcgggggcgccggtaaggcc

SEQ ID NO: 29 - DNA, 150 bp, Mycobacterium tuberculosis
cctcgggcagctcgaacagatcgaacgacgggtttccggcccatccgaccatcttggagccctcctaatctccgggctagtcgcgggttaacttacccggcggccgctccacttccgcatccttggccgccacgccgtcggcgagccggt

SEQ ID NO: 30 - DNA, 57 bp, Mycobacterium tuberculosis
gtcatgctgatcgcggtgctcgctcggctgatgatgcgcggctggcggcgccgttcg

SEQ ID NO: 31 - DNA, 57 bp, Mycobacterium tuberculosis
cgaacggcgccgccagccgcgcatcatcagccgagcgagcaccgcgatcagcatgac

SEQ ID NO: 32 - DNA, 77 bp, Mycobacterium tuberculosis
tgcatggctgtcgtcagctcgtgtcgtgagatgttgggttaagtcccgcaacgagcgcaacccttgtcattagttgc

SEQ ID NO: 33 - DNA, 71 bp, Mycobacterium tuberculosis
ccggctggcgcagccgatacacctcgcgccacatcatgccggcctcgaagtcggctccggcgaacgacttt

SEQ ID NO: 34 - DNA, 71 bp, Mycobacterium tuberculosis
aaagtcgttcgccggagccgacttcgaggccggcatgatgtggcgcgaggtgtatcggctgcgccagccgg

SEQ ID NO: 35 - DNA, 150 bp, Mycobacterium tuberculosis
acccgcggatacgggtccggtaccgccaaccgcggcgccgacggccgccggcgccgcggcggcagcttcggcggcctcgggaccgatccatcccagcgcccgccacgatgtaataaggctgttgccaataccaatggcgaaatatggcaa

SEQ ID NO: 36 - DNA, 150 bp, Mycobacterium tuberculosis
ccggggtggctggaatggttcatcaactggtatctgccgatatcacagctgttttacaacaccgtgggtttgccatatttcgccattggtattggcaacagccttattacatcgtggcgggcgctgggatggatcggtcccgaggccgcc

SEQ ID NO: 37 - DNA, 111 bp, Mycobacterium tuberculosis
caggtagccggtcagccggattgggtctcgttgcgccggcaggtgacggtcgcgcagcgaaaaagcgacctgcgggccgccgaggatccgatcgacgccgtcgtatgcgcc

SEQ ID NO: 38 - DNA, 111 bp, Mycobacterium tuberculosis
ggcgcatacgacggcgtcgatcggatcctcggcggcccgcaggtcgctttttcgctgcgcgaccgtcacctgccggcgcaacgagacccaatccggctgaccggctacctg

SEQ ID NO: 39 - DNA, 150 bp, Mycobacterium tuberculosis
tcacatccgggtggcgggcagccgtcgtctgcaacgaggccgacggtaggcgatgggtgaggccatggagggcaaatgtccccatggtgtatcgattacgtaccctcttacttcgtcggggtcgcatcggcattcgccttgcccgcctgt

SEQ ID NO: 40 - DNA, 150 bp, Mycobacterium tuberculosis
cgggcgacacaccgtcccgatacatgtcagcaaccgggtcgatcgtggtgaatgcacaggcgggcaaggcgaatgccgatgcgaccccgacgaagtaagagggtacgtaatcgatacaccatggggacatttgccctccatggcctcacc

SEQ ID NO: 41 - DNA, 150 bp, Mycobacterium tuberculosis
accggtgtcttctgccatcaacaggcctcgaccgcggggcagcgggccgcccttcatcttgccgcgaatgaagccctcgtcgggatcggcgtccatcaccagcagtggcgcattggcctgatgcagggcccgcaacatcgggtcgctgcc

SEQ ID NO: 42 - DNA, 150 bp, Mycobacterium tuberculosis
agccggcagcgacccgatgttgcgggccctgcatcaggccaatgcgccactgctggtgatggacgccgatcccgacgagggcttcattcgcggcaagatgaagggcggcccgctgccccgcggtcgaggcctgttgatggcagaagacac

SEQ ID NO: 43 - DNA, 69 bp, Mycobacterium tuberculosis
agaacaccgtcgaatgcatgcaagccggtgcggtgttcggcttcgccgggctggtagacgggttggtag

SEQ ID NO: 44 - DNA, 69 bp, Mycobacterium tuberculosis
ctaccaacccgtctaccagcccggcgaagccgaacaccgcaccggcttgcatgcattcgacggtgttct

SEQ ID NO: 45 - DNA, 112 bp, Mycobacterium tuberculosis
cggggtcatggtcgctctatgcctcggcggcggcgttttcgggctgagcctcggcaagcacgtcacgcagagcggcttctacgacgacggcagccaatcggtgcaagcatcg

SEQ ID NO: 46 - DNA, 112 bp, Mycobacterium tuberculosis
cgatgcttgcaccgattggctgccgtcgtcgtagaagccgctctgcgtgacgtgcttgccgaggctcagcccgaaaacgccgccgccgaggcatagagcgaccatgaccccg

SEQ ID NO: 47 - DNA, 150 bp, Mycobacterium tuberculosis
acgatctgccgggcggcggcatgtgtgcgcggcgcgatgccatcgacgaggcgcgccaaccgcggcgccggaaccgccaggatgacggcgtcggcctgccagcggccgccggtttcgtcgcgcagcacccagccgcgttcgagctggacc

SEQ ID NO: 48 - DNA, 150 bp, Mycobacterium tuberculosis
acgatctgccgggcggcggcatgtgtgcgcggcgcgatgccatcgactaggcgcgccaaccgcggcgccggaaccgccaggatgacggcgtcggcctgccagcggccgccggtttcgtcgcgcagcacccagccgcgttcgagctggacc

SEQ ID NO: 49 - DNA, 150 bp, Mycobacterium tuberculosis; identical to SEQ ID NO: 39

SEQ ID NO: 50 - DNA, 150 bp, Mycobacterium tuberculosis; identical to SEQ ID NO: 40

SEQ ID NO: 51 - DNA, 114 bp, Mycobacterium tuberculosis
gcacgcacccacctgcacatcgaaatcgtgcccggcctggccgccagcagcgcggtcccgacctatgccgggttgccgctgggttcgtcgcacaccgtcgccgacgtgcgtatc

SEQ ID NO: 52 - DNA, 114 bp, Mycobacterium tuberculosis
gatacgcacgtcggcgacggtgtgcgacgaacccagcggcaacccggcataggtcgggaccgcgctgctggcggccaggccgggcacgatttcgatgtgcaggtgggtgcgtgc

SEQ ID NO: 53 - DNA, 114 bp, Mycobacterium tuberculosis; identical to SEQ ID NO: 51

SEQ ID NO: 54 - DNA, 114 bp, Mycobacterium tuberculosis; identical to SEQ ID NO: 52

SEQ ID NO: 55 - DNA, 150 bp, Mycobacterium tuberculosis
ccacgtggctacccatgaccgcgctctcagcgatcttgtctttcttgacgtagtggaagtatttcggtgtgtcgctgccggtcccgtcgtcgacgcgttcgtcggcgtcggtacgttcaatcgtctgggtctgcatacctgacattgtgc

SEQ ID NO: 56 - DNA, 150 bp, Mycobacterium tuberculosis
tgccaagggcacaatgtcaggtatgcagacccagacgattgaacgtaccgacgccgacgaacgcgtcgacgacgggaccggcagcgacacaccgaaatacttccactacgtcaagaaagacaagatcgctgagagcgcgggcatgggtag

SEQ ID NO: 57 - DNA, 64 bp, Mycobacterium tuberculosis
cttccgataaatagttagccgaattatatcctcaggcatcaatctcagctcgtccaatcgagca

SEQ ID NO: 58 - DNA, 64 bp, Mycobacterium tuberculosis
tgctcgattggacgagctgagattgatgcctgaggatataattcggctaactatttatcggaag

SEQ ID NO: 59 - DNA, 89 bp, Mycobacterium tuberculosis
tgatggcgctggtggaatactccgccgacgaaatcagagaagtgttctccgacttccccgatctggaggtgtgtgtctacgccgcgccc

SEQ ID NO: 60 - DNA, 89 bp, Mycobacterium tuberculosis
ggacgcggcgtagacacacacctccagatcggggaagtcggagaacacttctctgatttcgtcggcggagtattccaccagcgccatca

SEQ ID NO: 61 - DNA, 93 bp, Mycobacterium tuberculosis
ggtcgtgctggcggccacgccgtgatcgccagctcgaagacaacgcgccacgcgtgaagcggctcgatctggtcgccgggcccaacggcgccg

SEQ ID NO: 62 - DNA, 93 bp, Mycobacterium tuberculosis
cggcgccgttgggcccggcgaccagatcgagccgcttcacgcgtggcgcgttgtcttcgagctggcgatcacggcgtggccgccagcacgacc

SEQ ID NO: 63 - DNA, 150 bp, Mycobacterium tuberculosis
caaaggattaccagcgcgaggacctgaaccctgagttcttcgcggcgtgttctcggcatctgcatggacgtagcagactgtggttgttccgctaccagggcacgccaattgccttctttttgaacgtttggggtgcggatgagaactaca

SEQ ID NO: 64 - DNA, 150 bp, Mycobacterium tuberculosis
cagtatgtagttctcatccgcaccccaaacgttcaaaaagaaggcaattggcgtgccctggtagcggaacaaccacagtctgctacgtccatgcagatgccgagaacacgccgcgaagaactcagggttcaggtcctcgcgctggtaatc

SEQ ID NO: 65 - DNA, 150 bp, Mycobacterium tuberculosis
cgagggtgacgacccgtcgatgaccaacccctacatgtacgacgtcgtcgacaccaagcgcggggcccgcaaaagctacaccgaagccctgatcggacgtggcgacatctcgatgaaagaggccgaggacgcgctgcgcgactaccaggg

SEQ ID NO: 66 - DNA, 104 bp, Mycobacterium tuberculosis
ttagcccaccgtatcgggcggcggggacttgccggaaaccgacgaacgcctcggcatccatccaaatggcgacgaaagatcacggaattgtcgcgaaacgaacg

SEQ ID NO: 67 - DNA, 82 bp, Mycobacterium tuberculosis
cctcgagcaaaaacgtcttcaccggtgttgcccgcacccgtagccgccaacaacgcggcatccagatcgcgctgttggctgg

SEQ ID NO: 68 - DNA, 82 bp, Mycobacterium tuberculosis
ccagccaacagcgcgatctggatgccgcgttgttggcggctacgggtgcgggcaacaccggtgaagacgtttttgctcgagg

SEQ ID NO: 69 - DNA, 150 bp, Mycobacterium tuberculosis
caaaggattaccagcgcgaggacctgaaccctgagttcttcgcggcgtgttctcggcatctgcatggacgtagcagactgtggttgttccgctaccagggcacgccaattgccgtctttttgaacggttggggtgcggatgagacctaca

SEQ ID NO: 70 - DNA, 150 bp, Mycobacterium tuberculosis
cagtatgtagttctcatccgcaccccaaacgttcaaaaagaaggcaattggcgtgccctggtagcggaacaaccacagtctgctacgtccatgcagatgccgagaacacgccgcgaagaactcaggggtcaggtcctcgcgctggtaatc

SEQ ID NO: 71 - DNA, 150 bp, Mycobacterium tuberculosis
ggctaggcgcgaaaagcgcgtgttcgacatgttcggagatcgcatgaataaaaacgatgtgagattcctggattcgcccggtgtcgcgtgacgggacgttgatcaagaaatctgcgaattctgccagctggccgccggagtcgcccgtca

SEQ ID NO: 72 - DNA, 83 bp, Mycobacterium tuberculosis
ccacttcgggttcaccctcggcggcaacgagcgtgtcgagcatccgttcacactgcgccgcggtgatggcgtagccgtcgggt

SEQ ID NO: 73 - DNA, 83 bp, Mycobacterium tuberculosis
acccgacggctacgccatcaccgcggcgcagtgtgaacggatgctcgacacgctcgttgccgccgagggtgaacccgaagtgg

SEQ ID NO: 74 - DNA, 18 bp, Artificial Sequence
  Synthetic sequence
ggctgtgggtagcagacc

SEQ ID NO: 75 - DNA, 23 bp, Artificial Sequence
  Synthetic sequence
acctgaaagacgttatccaccat

SEQ ID NO: 76 - DNA, 18 bp, Artificial Sequence
  Synthetic sequence
cgggtccagatggcttgc

SEQ ID NO: 77 - DNA, 22 bp, Artificial Sequence
  Synthetic sequence
cggctagtgcattgtcatagga

SEQ ID NO: 78 - DNA, 21 bp, Artificial Sequence
  Synthetic sequence
tgtcgacctgggcagggttcg

SEQ ID NO: 79 - DNA, 22 bp, Artificial Sequence
  Synthetic sequence
tccgaccgcgctccgaccgacg

SEQ ID NO: 80 - DNA, 150 bp, Artificial Sequence
  Synthetic sequence
ttagcagcgacgaagatcatgcgctcacgctctcggtgtcctcattcatcagttattcacaacgctatgctgtaactcgacctgacaagactgtacctatgagaaggcacttgctaccttatgcaagcgtcagcccgcggtatcgcttgg

SEQ ID NO: 81 - DNA, 20 bp, Artificial Sequence
  Synthetic sequence
cgctctcggtgtcctcattc

SEQ ID NO: 82 - DNA, 20 bp, Artificial Sequence
  Synthetic sequence
ggctgacgcttgcataaggt

SEQ ID NO: 83 - DNA, 29 bp, Artificial Sequence
  Synthetic sequence
  misc_feature (1)..(1): VIC fluorescent dye (VIC)
  misc_feature (29)..(29): Carboxytetramethylrhodamine (TAMRA)
cacaacgctatgctgtaactcgacctgac

SEQ ID NO: 84 - DNA, 21 bp, Artificial Sequence
  Synthetic sequence
  misc_feature (1)..(1): Carboxyfluorescein (FAM)
  misc_feature (21)..(21): Carboxytetramethylrhodamine (TAMRA)
tgtcgacctgggcagggttcg

SEQ ID NO: 85 - DNA, 22 bp, Artificial Sequence
  Synthetic sequence
  misc_feature (1)..(1): Fluorescein (FAM)
  misc_feature (22)..(22): Carboxytetramethylrhodamine (TAMRA)
tccgaccgcgctccgaccgacg

SEQ ID NO: 86 - DNA, 20 bp, Artificial Sequence
  Synthetic sequence
tcatcatcatccgggtctcc

SEQ ID NO: 87 - DNA, 18 bp, Artificial Sequence
  Synthetic sequence
gctcaccatctgggccac

SEQ ID NO: 88 - DNA, 34 bp, Artificial Sequence
  Synthetic sequence
cctccaggtagaaggccatttttccaccctgtag

SEQ ID NO: 89 - DNA, 29 bp, Artificial Sequence
  Synthetic sequence
cacaacgctatgctgtaactcgacctgac